(19) United States
(12) Patent Application Publication
(10) Pub. No.: US 2009/0318430 A1
(43) Pub. Date: Dec. 24, 2009

(54) MEDICAL USE OF CYCLIN DEPENDENT KINASES INHIBITORS

(75) Inventors: Andrew Pike, Bishops Stortford (GB); John Francis Lyons, London (GB)

Correspondence Address: HESLIN ROTHENBERG FARLEY & MESITI PC, 5 COLUMBIA CIRCLE, ALBANY, NY, 12203, US

(73) Assignee: ASTEX THERAPEUTICS LIMITED, Cambridge (GB)

(21) Appl. No.: 12/374,461

(22) PCT Filed: Jul. 20, 2007
(86) PCT No.: PCT/GB2007/002753

§ 371 Date: Jun. 10, 2009

Related U.S. Application Data
(60) Provisional application No. 60/832,678, filed on Jul. 21, 2006.

(51) Int. Cl. A61K 31/541; A61K 31/454; A61K 31/5377; A61K 31/415

(52) U.S. Cl. 514/227.8; 514/326; 514/236.5; 514/404

(57) ABSTRACT

The invention provides the use of a compound for the manufacture of a medicament for the treatment of pain, wherein the compound is a compound of the formula (0):

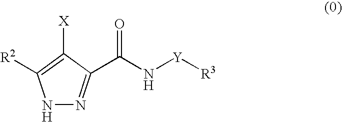

or a salt or tautomers or N-oxides or solvate thereof; wherein X is a group R¹-A-NR⁴— or a 5- or 6-membered carbocyclic or heterocyclic ring; A is a bond, SO₂, C═O, NR^{g}(C═O) or O(C═O) wherein R^{g} is hydrogen or C₁₋₄ hydrocarbyl optionally substituted by hydroxy or C₁₋₄ alkoxy; Y is a bond or an alkylene chain of 1, 2 or 3 carbon atoms in length; R¹ is hydrogen; a carbocyclic or heterocyclic group having from 3 to 12 ring members; or a C₁₋₈ hydrocarbyl group optionally substituted by one or more substituents selected from halogen, hydroxy, C₁₋₄ hydrocarbyloxy, amino, mono- or di-C₁₋₄ hydrocarbylamino, and carbocyclic or heterocyclic groups having from 3 to 12 ring members, and wherein 1 or 2 of the carbon atoms of the hydrocarbyl group may optionally be replaced by an atom or group selected from O, S, NH, SO, SO₂; R² is hydrogen; halogen; C₁₋₄ alkoxy; or a C₁₋₄ hydrocarbyl group optionally substituted by halogen, hydroxyl or C₁₋₄ alkoxy (e.g. methoxy); R³ is selected from hydrogen and carbocyclic and heterocyclic groups having from 3 to 12 ring members; and R⁴ is hydrogen or a C₁₋₄ hydrocarbyl group optionally substituted by halogen (e.g. fluorine), hydroxyl or C₁₋₄ alkoxy.

The invention also provides the use of the compounds of the formula (0) for the treatment of stroke and for the treatment of polycystic kidney disease.

### TECHNICAL FIELD

This invention relates to pyrazole amide compounds for use in the prophylaxis or treatment of pain and methods for the prophylaxis or treatment of pain. The invention also provides compounds for the treatment of stroke and for use as neuroprotective agents as well as methods of treating stroke and methods of neuroprotection following stroke. The invention further provides compounds for use in the treatment of polycystic kidney disease and methods for treating polycystic kidney disease.

### BACKGROUND OF THE INVENTION

Protein kinases constitute a large family of structurally related enzymes that are responsible for the control of a wide variety of signal transduction processes within the cell (Hardie, G. and Hanks, S. (1995) The Protein Kinase Facts Book. I and II, Academic Press, San Diego, Calif.). The kinases may be categorized into families by the substrates they phosphorylate (e.g., protein-tyrosine, protein-serine/threonine, lipids, etc.). Sequence motifs have been identified that generally correspond to each of these kinase families (e.g., Hanks, S. K., Hunter, T., FASEB J., 9:576-596 (1995); Knighton, et al., Science, 253:407-414 (1991); Hiles, et al., Cell, 70:419-429 (1992); Kunz, et al., Cell, 73:585-596 (1993); Garcia-Bustos, et al., EMBO J., 13:2352-2361 (1994)).

Protein kinases may be characterized by their regulation mechanisms. These mechanisms include, for example, autophosphorylation, transphosphorylation by other kinases, protein-protein interactions, protein-lipid interactions, and protein-polynucleotide interactions. An individual protein kinase may be regulated by more than one mechanism.

Kinases regulate many different cell processes including, but not limited to, proliferation, differentiation, apoptosis, motility, transcription, translation and other signalling processes, by adding phosphate groups to target proteins. These phosphorylation events act as molecular on/off switches that can modulate or regulate the target protein biological function. Phosphorylation of target proteins occurs in response to a variety of extracellular signals (hormones, neurotransmitters, growth and differentiation factors, etc.), cell cycle events, environmental or nutritional stresses, etc. The appropriate protein kinase functions in signalling pathways to activate or inactivate (either directly or indirectly), for example, a metabolic enzyme, regulatory protein, receptor, cytoskeletal protein, ion channel or pump, or transcription factor. Uncontrolled signalling due to defective control of protein phosphorylation has been implicated in a number of diseases, including, for example, inflammation, cancer, allergy/asthma, diseases and conditions of the immune system, diseases and conditions of the central nervous system, and angiogenesis.

The process of eukaryotic cell division may be broadly divided into a series of sequential phases termed G1, S, G2 and M. Correct progression through the various phases of the cell cycle has been shown to be critically dependent upon the spatial and temporal regulation of a family of proteins known as cyclin dependent kinases (cdks) and a diverse set of their cognate protein partners termed cyclins. Cdks are cdc2 (also known as cdk1) homologous serine-threonine kinase proteins that are able to utilise ATP as a substrate in the phosphorylation of diverse polypeptides in a sequence dependent context. Cyclins are a family of proteins characterised by a homology region, containing approximately 100 amino acids, termed the “cyclin box” which is used in binding to, and defining selectivity for, specific cdk partner proteins.

Modulation of the expression levels, degradation rates, and activation levels of various cdks and cyclins throughout the cell cycle leads to the cyclical formation of a series of cdk/cyclin complexes, in which the cdks are enzymatically active. The formation of these complexes controls passage through discrete cell cycle checkpoints and thereby enables the process of cell division to continue. Failure to satisfy the pre-requisite biochemical criteria at a given cell cycle checkpoint, i.e. failure to form a required cdk/cyclin complex, can lead to cell cycle arrest and/or cellular apoptosis. Aberrant cellular proliferation, as manifested in cancer, can often be attributed to loss of correct cell cycle control. Inhibition of cdk enzymatic activity therefore provides a means by which abnormally dividing cells can have their division arrested and/or be killed. The diversity of cdks, and cdk complexes, and their critical roles in mediating the cell cycle, provides a broad spectrum of potential therapeutic targets selected on the basis of a defined biochemical rationale.

Although most cdks have been implicated in regulation of the cell cycle there is evidence that certain members of the cdk family are involved in other biochemical processes. This is exemplified by cdk5 which is necessary for correct neuronal development and which has also been implicated in the phosphorylation of several neuronal proteins such as Tau, NUDE-1, synapsin1, DARPP32 and the Muncl8/Syntaxin1A complex. Neuronal cdk5 is conventionally activated by binding to the p35/p39 proteins. Cdk5 activity can, however, be deregulated by the binding of p25, a truncated version of p35. Conversion of p35 to p25, and subsequent deregulation of cdk5 activity, can be induced by ischemia, excitotoxicity, and β-amyloid peptide. Consequently p25 has been implicated in the pathogenesis of neurodegenerative diseases, such as Alzheimer's, and is therefore of interest as a target for therapeutics directed against these diseases.

Cdk5 has been shown to have a role in mediating pain signalling. Cdk5 requires activation by p35 or its calpain cleavage product p25. Both Cdk5 and p35 have been shown to be expressed in nociceptive neurons. In p35 knockout mice, which show substantially reduced Cdk5 activity, the response to painful thermal stimuli is delayed (Pareek, T. K., et al., Proceedings of the National Academy of Sciences., 103:791-796 (2006). Additionally administration of the cyclin-dependent kinase 5 (Cdk5) inhibitor roscovitine has been shown to attenuate the formalin-induced nociceptive responses in rats (Wang, Cheng-haung, et al., Acta Pharmacologica Sinica., 26:46-50 (2005). Activation of calpain is calcium dependent and is known to affected by activation of the NMDA receptor calcium channel (Amadoro, G; Proceedings of the National Academy of Sciences of the United States of America, 103, 2892-2897 (2006)). NMDA receptor antagonists are know to be clinically effective against neuropathic pain conditions (Christoph, T; et al., Neuropharmacology, 51, 12-17 (2006)). This efficacy may be linked to the effect of NMDA receptor related calcium influx on calpain activity and its subsequent effect on the activity of Cdk5. As such compounds inhibiting Cdk5 will be useful for the treatment or prevention of pain.

It is desirable to have an agent for the palliative treatment of pain, i.e. the direct relief of pain in addition to the relief of pain as the result of amelioration of the underlying disease or medical condition, which is the cause of the pain.

Various Cdk's (especially Cdk's 4, 5 & 6) have been shown to be involved with or mediate neuronal death following hypoxic or ischemic insult (Rashidan, J.; et al.; Proceedings of the National Academy of Sciences., 102:14080-14085 (2005). Furthermore the Cdk inhibitor flavopiridol has been shown to significantly reduce neuronal death in a rat model of focal cerebral ischemia (Osuga, H.; et al.; Proceedings of the National Academy of Sciences., 97:10254-10259 (2000). Cdk5 inhibitors have been shown to have protective effects in both necrotic and apoptotic paradigms of neuronal cell death (Weishaupt, J.; et al.; Molecular and Cellular Neuroscience., 24:489-502 (2003). Based on these observations it is expected that inhibitors of Cdk's, especially Cdk's 4, 5 and 6, will have neuroprotective effects following cerebrovascular events in the brain and other instances where damage may be induced due to hypoxia.

Stroke is a cerebrovascular event, which occurs when the normal bloodflow to the brain is disrupted, and the brain receives too much or too little blood. Stroke is one of the leading causes of death worldwide, and is also one of the most common causes of neurologic disability.

Ischemic stroke, which is the most common type of stroke, results from insufficient cerebral circulation of blood caused by obstruction of the inflow of arterial blood. Normally, adequate cerebral blood supply is ensured by a system of arteries within the brain. However, various disorders, including inflammation and atherosclerosis, can cause a thrombus, i.e., a blood clot that forms in a blood vessel. The thrombus may interrupt arterial blood flow, causing brain ischemia and consequent neurologic symptoms. Ischemic stroke may also be caused by the lodging of an embolus (an air bubble) from the heart in an intracranial vessel, causing decreased perfusion pressure or increased blood viscosity with inadequate cerebral blood flow. An embolus may be caused by various disorders, including atrial fibrillation and atherosclerosis.

A second type of stroke, hemorrhagic stroke, involves a hemorrhage or rupture of an artery leading to the brain. Hemorrhagic stroke results in bleeding into brain tissue, including the epidural, subdural, or subarachnoid space of the brain. A hemorrhagic stroke typically results from the rupture of an arteriosclerotic vessel that has been exposed to arterial hypertension or to thrombosis.

One opportunity for intervention in stroke is the prevention or reduction of risk of stroke in patients at risk for stroke. There are many known risk factors for stroke, including vascular inflammation, atherosclerosis, arterial hypertension, diabetes, hyperlipidemia and atrial fibrillation. At risk patients have been treated with agents to control blood pressure or manage blood lipid level, and have been treated with antiplatelet agents (such as clopidrogel) and anticoagulants. A second opportunity is the treatment of acute stroke. However, current pharmacologic therapies for treating acute stroke are limited to restoring blood flow within a narrow therapeutic time window of less than three hours after stroke. There remains a need for agents which are effective within a longer therapeutic time window. Another opportunity is recovery or restoration after the acute stroke period, i.e. the reduction or prevention of secondary cell damage in the penumbra. There remains a need for agents which are effective in reducing or preventing secondary cell damage after stroke.

It would be desirable to obtain a single pharmaceutical agent which can be used in more than one of the above-mentioned opportunities for treating stroke. Such an agent may be administered to patients at risk for stroke, and also may be administered to patients suffering from acute stroke, or patients undergoing treatment for recovery or restoration after the acute stroke period. Such an agent may also target more than one distinct mechanism in the biochemical cascade of stroke.

There is also evidence that CDK inhibitors may be of use in treating renal diseases such as polycystic kidney disease.

Polycystic kidney disease (PKD) is the most prevalent hereditary renal disorder, accounting for over 5 percent of patients on chronic hemodialysis. PKD constitutes a subset of renal cystic disorders in which cysts are distributed throughout the cortex and/or medulla of the kidneys. Typically, the disease is characterized by the proliferation of epithelial cells, formation of renal cysts, liver cysts, intracranial aneurysm, severe dilations of collecting ducts, and progressive renal insufficiency. Renal cysts arise in the renal parenchyma, and begin as dilations or outpouchings from existing nephrons or collecting ducts or from the developmental counterparts of these structures. Renal cysts contain a fluid that presumably derives from their parent nephron and/or is a local secretion. The development of renal cysts may be hereditary, developmental, or acquired, and may occur in the cortex, medulla or both. For further details see, for example, Brenner & Rector, The Kidney, Fourth Edition, 1991, Vol. 11, pp. 1657-1659.

PKD can be inherited as an autosomal dominant (AD) or autosomal recessive (AR) trait but may also be found in association with a variety of clinical conditions or acquired at some point of life by a patient with an underlying, noncystic renal disease. In humans, autosomal dominant polycystic kidney disease (ADPKD) has a later onset and slower progression than autosomal recessive polycystic kidney disease (ARPKD), which usually affects newborns or young children. Adult PKD (ADPKD) affects approximately 500,000 Americans with about 7,000 new patients identified each year. Infants with ARPKD inherit a rapidly developing form, which can lead to renal insufficiency in the neonatal period.

ADPKD, which is the most common dominantly inherited kidney disease, usually appears in midlife, and is characterized morphologically by massive cyst enlargement, moderate interstitial infiltration with mononuclear cells, and extensive fibrosis. Characteristic symptoms include proteinuria, abdominal pain and palpable kidneys, followed by hematuria, hypertension, pyuria, uremia and calculi. In about 15% of patients, death is due to cerebral aneurysm. ADPKD is caused by mutations in one of three genes: PKD1 on chromosome 16 accounts for approximately 85% of cases whereas PKD2 on chromosome 4 accounts for approximately 15%. Mutations in the so far unmapped PKD3 gene are rare. (Reeders et al., Nature 317:542-544 [19851; Kimberling et al., Genomics 18:467-472 [19931; Daoust et al., Genomics 25:733-736 [19951; Koptides et al., Hum. Mol. Genet. 8:509-513 [19991). PKD 1, the gene that is mutated in approximately 85% of autosomal dominant polycystic kidney disease (ADPKD) cases in humans, has recently been identified (The European Polycystic Kidney Disease Consortium, 1994). Recent evidence has suggested that a two-hit mechanism, in which the normal PKD1 allele is also inactivated, may be required for cyst growth.

In ADPKD, the renal cysts remain small for 30-40 years. They then start to expand, progressively replacing normally functioning renal parenchyma. Factors involved in cyst expansion include loss of epithelial differentiation, disordered cellular proliferation and apoptosis, secretion of chloride and other ions into the cyst fluid and the development of inflammation around the outer circumference of the cyst wall (Grantham, J. Am J. Kid. Dis. 28:788-803 [19961). Currently, no therapies exist for ADPKD which accounts for 8-10% of patients requiring kidney transplantation or dialysis (Gabow P. A., 1993, N. Engl. J. Med. 329: 332-342).

ARPKD is a rare inherited disorder which usually becomes clinically manifest in early childhood, although presentation of ARPKD in later life has also been observed. ARPKD can cause massive bilateral enlargement of the kidneys. Most individuals surviving the neonatal period eventually develop renal failure. ARPKD was first studied in C57BL16J mice in whom it arises spontaneously (Prominger at al., J. Urol. 127:556-560 [19821). The cpk mutation characteristic of this disease has been mapped to mouse chromosome 12 (Davisson at al., Genomics 9:778-781 [19911). The gene responsible for ARPKD in humans has been mapped to chromosome 6p. More recently, fine mapping of the autosomal recessive polycystic kidney disease locus (PKHD 1) has been reported (Mucher at al., Genomics 48:40-45 [19981).

In view of the severity and frequency of occurrence of PKD, there is a need for the identification of treatments for the prevention and/or treatment of diseases involving cyst formation and cyst expansion.

The large number of genes showing abnormal expression in cystic kidneys from humans and rodents with PKD suggests that cellular processes associated with signal transduction, transcriptional regulation, and cell-cycle control are involved in cyst formation and that the cellular defect in PKD directly affects the regulation of epithelial differentiation (Calvet, 1998; Torres, 1998).

It has been reported that taxol and taxol derivatives inhibit the progression of PKD and prolongs the survival of polycystic cpk mice (Woo at al., Nature 368:750-753 [19941; PCT publication WO 94/08041).

The dysregulated cell cycle may be the most proximal cause of cystogenesis, and that intervention targeted at this point could provide significant therapeutic benefit for PKD. It has recently been shown that treatment with the cyclin-dependent kinase (CDK) inhibitor (R)-roscovitine yielded effective arrest of cystic disease in jck and cpk mouse models of PKD. Continuous daily administration of the drug was not required to achieve efficacy; pulse treatment provided a robust, long-lasting effect, indicating potential clinical benefits for a lifelong therapy. Molecular studies of the mechanism of action revealed effective cell-cycle arrest, transcriptional inhibition and attenuation of apoptosis. Moreover, it was discovered that roscovitine was active against cysts originating from different parts of the nephron, a desirable feature for the treatment of ADPKD, in which cysts form in multiple nephron segments. The results indicated that inhibition of CDK might afford a new and effective approach to the treatment of PKD.

CDK inhibitors are of interest as therapeutic agents in proliferative renal diseases primarily because of their ability to potently inhibit the activity of cell cycle CDKs, thereby directly inducing cell cycle arrest of proliferating cells (Nelson, P. J. and Shankland, S. J. Therapeutics in renal disease: the road ahead for antiproliferative targets. Nephron Exp Nephrol 2006, 103: e6-e15).

In addition, it has also been proposed in Drug News Perspect. 2006 July-August; 19(6):325-8) that inhibition of CDK kinases may provide a novel therapy for a variety of proliferative renal diseases. Furthermore, in an article in Nature (Nature, 2006, vol. 444, 949-952), it has been reported that in vitro studies indicated that the CDK inhibitor CYC202 may have therapeutic potential in the treatment of polycystic kidney diseases. WO 2005/012256 (Astex Technology Limited) discloses various compounds of formula (0) (see below) having activity as inhibitors of various kinases for use in the treatment of disease states and conditions such as cancer.

WO 2006/077426 (Astex Therapeutics Limited) discloses various compounds and salts of formula (0) having activity as inhibitors of cyclin dependent kinases, and glycogen synthase kinase-3.

WO 2006/077416 (Astex Therapeutics Limited) discloses various compounds of formula (I′″) having activity as inhibitors of cyclin dependent kinases, and glycogen synthase kinase.

### SUMMARY OF THE INVENTION

It has now been found that compounds of the formula (0) have good activity against Cdk5 kinase and, on the basis of such activity, the compounds will be useful in the treatment of pain.

Accordingly, in a first aspect, the invention provides the use of a compound for the manufacture of a medicament for the treatment of pain, wherein the compound is a compound of the formula (0):

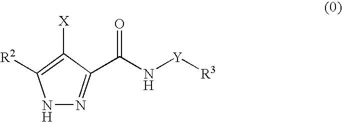

or a salt or tautomer or N-oxide or solvate thereof;
wherein
X is a group R¹-A-NR⁴— or a 5- or 6-membered carbocyclic or heterocyclic ring;
A is a bond, SO₂, C═O, NR^{g}(C═O) or O(C═O) wherein R^{g} is hydrogen or C₁₋₄ hydrocarbyl optionally substituted by hydroxy or C₁₋₄ alkoxy;
Y is a bond or an alkylene chain of 1, 2 or 3 carbon atoms in length;
R¹ is hydrogen; a carbocyclic or heterocyclic group having from 3 to 12 ring members; or a C₁₋₈ hydrocarbyl group optionally substituted by one or more substituents selected from halogen (e.g. fluorine), hydroxy, C₁₋₄ hydrocarbyloxy, amino, mono- or di-C₁₋₄ hydrocarbylamino, and carbocyclic or heterocyclic groups having from 3 to 12 ring members, and wherein 1 or 2 of the carbon atoms of the hydrocarbyl group may optionally be replaced by an atom or group selected from O, S, NH, SO, SO₂;
R² is hydrogen; halogen; C₁₋₄ alkoxy (e.g. methoxy); or a C₁₋₄ hydrocarbyl group optionally substituted by halogen (e.g. fluorine), hydroxyl or C₁₋₄ alkoxy (e.g. methoxy);
R³ is selected from hydrogen and carbocyclic and heterocyclic groups having from 3 to 12 ring members; and
R⁴ is hydrogen or a C₁₋₄ hydrocarbyl group optionally substituted by halogen (e.g. fluorine), hydroxyl or C₁₋₄ alkoxy (e.g. methoxy).

The compounds of formula (0) correspond to formula (0) in WO 2005/012256 (PCT/GB2004/003179) and it is to be understood that references to formula (0) herein include each of the various possible substituents, sub-groups, embodiments and examples thereof as defined in WO 2005/012256. In particular the definitions of the groups are as defined at pages 23-37 in WO 2005/012256. Specific embodiments of and preferences for X, Y, A, R^{g}, R¹ to R⁴ and R¹⁰ are detailed at pages 37 to 81 of WO 2005/012256.

Particular and preferred compounds of formula (0) and sub-groups thereof are as set out in the claims appended hereto and as set out in the claims and examples of WO 2005/012256.

In one preferred subgroup of compounds within formula (0), the compounds have the formula (I′″):

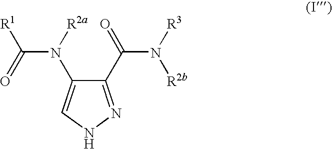

or is a salt, tautomer, solvate or N-oxide thereof;
wherein:
R¹ is 2,6-dichlorophenyl;
R^{2a} and R^{2b} are both hydrogen;
and R³ is a group:

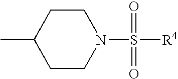

where R⁴ is C₁₋₄ alkyl.

In another aspect, the invention provides the use of a compound of the formula (0) or a sub-group thereof such as formula (I′″) for the manufacture of a medicament for the prophylaxis or treatment of stroke.

In a further aspect, the invention provides the use of a compound of the formula (0) or a subgroup thereof such as formula (I′″) for the manufacture of a medicament for use as a neuroprotective agent.

In a further aspect, the invention provides the use of a compound of the formula (0) or a subgroup thereof such as formula (I′″) for the manufacture of a medicament for use in the treatment or prophylaxis of polycystic kidney disease. In other aspects, the invention provides:
A compound of the formula (0) or a subgroup thereof such as formula (I′″) for use in the treatment of pain.
A compound of the formula (0) or a subgroup thereof such as formula (I′″) for use in the reduction or elimination of pain in a patient (e.g. a mammal such as a human) suffering from pain.
The use of a compound of the formula (0) or a subgroup thereof such as formula (I′″) for the manufacture of a medicament for use in the reduction or elimination of pain in a patient (e.g. a mammal such as a human) suffering from pain.
The use of a compound of the formula (0) or a subgroup thereof such as formula (I′″) for the manufacture of a medicament for the treatment of any one or more of nociception, somatic pain, visceral pain, acute pain, chronic pain, hyperalgesia, allodynia, post operative pain, pain due to hypersensivity, headache, inflammatory pain (rheumatic, dental, dysmenorrhoea or infection), neurological pain, musculoskeletal pain, cancer related pain or vascular pain.
A compound of the formula (0) or a subgroup thereof such as formula (I′″) for use in treating any one or more of nociception, somatic pain, visceral pain, acute pain, chronic pain, hyperalgesia, allodynia, post operative pain, pain due to hypersensivity, headache, inflammatory pain (rheumatic, dental, dysmenorrhoea or infection), neurological pain, musculoskeletal pain, cancer related pain or vascular pain.
A method of treating pain in a patient such as a mammal (e.g. human), which method comprises administering to the patient a therapeutically effective amount of a compound of the formula (0) or a subgroup thereof such as formula (I′″).
A method for the reduction or elimination of pain in a patient (e.g. a mammal such as a human) suffering from pain, which method comprises administering to the patient an effective pain-reducing or pain-eliminating amount of a compound of the formula (0) or a subgroup thereof such as formula (I′″).
A method for the treatment of any one or more of nociception, somatic pain, visceral pain, acute pain, chronic pain, hyperalgesia, allodynia, post operative pain, pain due to hypersensivity, headache, inflammatory pain (rheumatic, dental, dysmenorrhoea or infection), neurological pain, musculoskeletal pain, cancer related pain or vascular pain, which method comprises administering to the patient a therapeutically effective amount of a compound of the formula (0) or a subgroup thereof such as formula (I′″).
A compound of the formula (0) or a subgroup thereof such as formula (I′″) for use in the prophylaxis or treatment of stroke.
A method for the prophylaxis or treatment of stroke in a patient such as a mammal (e.g. human), which method comprises administering to the patient a therapeutically effective amount of a compound of the formula (0) or a subgroup thereof such as formula (I′″).
A compound of the formula (0) or a subgroup thereof such as formula (I′″) for use as a neuroprotective agent.
A method of preventing or reducing neuronal damage in a patient suffering from stroke, which method comprises administering to the patient an effective neuroprotective amount of a compound of the formula (0) or a subgroup thereof such as formula (I′″).
The use of a compound of the formula (0) or a subgroup thereof such as formula (I′″) for the manufacture of a medicament for the prevention or reduction of risk of stroke in patients at risk for stroke, for example a patient exhibiting any one or more risk factors selected from vascular inflammation, atherosclerosis, arterial hypertension, diabetes, hyperlipidemia and atrial fibrillation.
A compound of the formula (0) or a subgroup thereof such as formula (I′″) for the prevention or reduction of risk of stroke in patients at risk for stroke, for example a patient exhibiting any one or more risk factors selected from vascular inflammation, atherosclerosis, arterial hypertension, diabetes, hyperlipidemia and atrial fibrillation.
A method for the prevention or reduction of risk of stroke in patients at risk for stroke, for example a patient exhibiting any one or more risk factors selected from vascular inflammation, atherosclerosis, arterial hypertension, diabetes, hyperlipidemia and atrial fibrillation, which method comprises administering to the patient an effective therapeutic amount of compound of the formula (0) or a subgroup thereof such as formula (I′″).
A compound of the formula (0) or a subgroup thereof such as formula (I′″) for use in the prophylaxis or treatment of polycystic kidney disease.
A method for the prophylaxis or treatment of polycystic kidney disease in a patient such as a mammal (e.g. human), which method comprises administering to the patient a therapeutically effective amount of a compound of the formula (0) or a subgroup thereof such as formula (I′″).
A compound of the formula (0) or a subgroup thereof such as formula (I′″) for use in the prevention or treatment of cyst formation in a mammalian (e.g. human) body.
The use of a compound of the formula (0) or a subgroup thereof such as formula (I′″) for the manufacture of a medicament for use in the prevention or treatment of cyst formation in a mammalian (e.g. human) body.
A method for the prophylaxis or treatment of cyst formation in a patient such as a mammal (e.g. human), which method comprises administering to the patient a therapeutically effective amount of a compound of the formula (0) or a subgroup thereof such as formula (I′″).
A compound of the formula (0) or a subgroup thereof such as formula (I′″) for use in the prophylaxis or treatment of cyst formation in a mammal (e.g. human).
A method for preventing or slowing down the progression of polycystic kidney disease in a patient such as a mammal (e.g. human), which method comprises administering to the patient a therapeutically effective amount of a compound of the formula (0) or a subgroup thereof such as formula (I′″).
A compound of the formula (0) or a subgroup thereof such as formula (I′″) for use in preventing or slowing down the progression of polycystic kidney disease.
The use of a compound of the formula (0) or a subgroup thereof such as formula (I′″) for the manufacture of a medicament for use in preventing or slowing down the progression of polycystic kidney disease.
A method for preventing or slowing down the development of a symptom of polycystic kidney disease (such as hypertension associated with PKD, bleeding into the cyst, or pain associated with cyst expansion) a patient such as a mammal (e.g. human), which method comprises administering to the patient a therapeutically effective amount of a compound of the formula (0) or a subgroup thereof such as formula (I′″).
A compound of the formula (0) or a subgroup thereof such as formula (I′″) for use in preventing or slowing down the development of a symptom of polycystic kidney disease (such as hypertension associated with PKD, bleeding into the cyst, or pain associated with cyst expansion).
The use of a compound of the formula (0) or a subgroup thereof such as formula (I′″) for the manufacture of a medicament for use in preventing or slowing down the development of a symptom of polycystic kidney disease (such as hypertension associated with PKD, bleeding into the cyst, or pain associated with cyst expansion).
A method for the treatment of progressive renal insufficiency associated with the progression of cystic kidney disease in a patient such as a mammal (e.g. human), which method comprises administering to the patient a therapeutically effective amount of a compound of the formula (0) or a subgroup thereof such as formula (I′″).
A compound of the formula (0) or a subgroup thereof such as formula (I′″) for use in the treatment of progressive renal insufficiency associated with the progression of cystic kidney disease.
The use of a compound of the formula (0) or a subgroup thereof such as formula (I′″) for the manufacture of a medicament for use in the treatment of progressive renal insufficiency associated with the progression of cystic kidney disease.
A method for the treatment of hypertension accompanying polycystic kidney disease in a patient such as a mammal (e.g. human), which method comprises administering to the patient a therapeutically effective amount of a compound of the formula (0) or a subgroup thereof such as formula (I′″).
A compound of the formula (0) or a subgroup thereof such as formula (I′″) for use in the treatment of hypertension accompanying polycystic kidney disease.
The use of a compound of the formula (0) or a subgroup thereof such as formula (I′″) for the manufacture of a medicament for use in the treatment of hypertension accompanying polycystic kidney disease.
A pharmaceutical composition for the treatment of a disease involving cyst formation or cyst expansion, comprising an effective amount of a compound of the formula (0) or a subgroup thereof such as formula (I′″) in admixture with a pharmaceutically acceptable carrier.
A compound of the formula (0) or (I′″) or any sub-groups or examples thereof as defined herein for use in the prophylaxis or treatment of a disease state or condition mediated by a cyclin dependent kinase 5.
The use of a compound of the formula (0) or (I′″) or any sub-groups or examples thereof as defined herein for the manufacture of a medicament for the prophylaxis or treatment of a disease state or condition mediated by a cyclin dependent kinase 5.
A method for the prophylaxis or treatment of a disease state or condition mediated by a cyclin dependent kinase 5, which method comprises administering to a subject in need thereof a compound of the formula (0) or (I′″) or any sub-groups or examples thereof as defined herein.
A method for alleviating or reducing the incidence of a disease state or condition mediated by a cyclin dependent kinase 5, which method comprises administering to a subject in need thereof a compound of the formula (0) or (I′″) or any sub-groups or examples thereof as defined herein.

### General Preferences and Definitions

In this specification, unless the context indicates otherwise, references to formula (0) include formulae (I), (I⁰), (Ia), (Ib), (II′), (III), (IV), (IVa), (Va), (Vb), (VIa), (VIb), (VII) or (VIII) as described in WO 2005/012256 and sub-groups, examples or embodiments of formulae (0), (I⁰), (Ia), (Ib), (II′), (III), (IV), (IVa), (Va), (Vb), (VIa), (VIb), (VII) or (VIII) as described in WO 2005/012256. Moreover, in this specification in general, unless the context indicates otherwise, references to a compound of formula (I′″) as described in WO 2006/077416 includes all subgroups of formula (I′″) as defined herein and the term ‘subgroups’ includes all preferences, embodiments, examples and particular compounds defined herein. Any references to formula (I′″) herein shall also be taken to refer to and any sub-group of compounds within formula (I′″) and any preferences and examples thereof unless the context requires otherwise.

In each of the foregoing paragraphs and elsewhere herein, the references to a compound of the formula (0) or (I′″) or any sub-groups or examples thereof also include within their scope any salts, solvates, tautomers or N-oxides of the compounds unless the context indicates otherwise.

In this specification, unless the context indicates otherwise, references to formula (0) are to be understood to include references to formulae (I⁰), (Ix) (I′″), (Ia), (Ib), (II′), (IV), (IVa), (Va), (VIa), (VIb) and all other sub-groups, preferences and examples thereof as defined herein.

In this specification, unless the context indicates otherwise, references to formula (I) are to be understood to include references to formulae (0), (I⁰), (Ix) (I′″), (Ia), (Ib), (II′), (IV), (IVa), (Va), (VIa), (VIb) and all other sub-groups, preferences and examples thereof as defined herein.

As used herein, the term “treatment” and the related terms “treat” and “treating” refer to both prophylactic or preventative treatment as well as curative or palliative treatment of pain. Thus, the term encompasses situations where pain is already being experienced by a subject or patient, as well as situations where pain is not currently being experienced but is expected to arise. The term “treatment”, “treat”, “treating” and related terms also cover both complete and partial pain reduction or prevention. Thus, for example, the compounds of the invention may prevent existing pain from worsening, or they reduce or even eliminate pain. When used in a prophylactic sense, the compounds may prevent any pain from developing or they may lessen the extent of pain that may develop.

As used herein, the term “modulation”, as applied to the activity of cyclin dependent kinase 5 (CDK5), is intended to define a change in the level of biological activity of the kinase(s). Thus, modulation encompasses physiological changes which effect an increase or decrease in the relevant kinase activity. In the latter case, the modulation may be described as “inhibition”. The modulation may arise directly or indirectly, and may be mediated by any mechanism and at any physiological level, including for example at the level of gene expression (including for example transcription, translation and/or post-translational modification), at the level of expression of genes encoding regulatory elements which act directly or indirectly on the levels of cyclin dependent kinase 5 (CDK5), or at the level of enzyme (e.g. cyclin dependent kinase 5 (CDK5) activity (for example by allosteric mechanisms, competitive inhibition, active-site inactivation, perturbation of feedback inhibitory pathways etc.). Thus, modulation may imply elevated/suppressed expression or over- or under-expression of the cyclin dependent kinase 5 (CDK5) including gene amplification (i.e. multiple gene copies) and/or increased or decreased expression by a transcriptional effect, as well as hyper- (or hypo-)activity and (de)activation of the cyclin dependent kinase 5 (CDK5) including (de)activation) by mutation(s). The terms “modulated”, “modulating” and “modulate” are to be interpreted accordingly.

As used herein, the term “mediated”, as used e.g. in conjunction with the cyclin dependent kinase 5 (CDK5) as described herein (and applied for example to various physiological processes, diseases, states, conditions, therapies, treatments or interventions) is intended to operate limitatively so that the various processes, diseases, states, conditions, treatments and interventions to which the term is applied are those in which cyclin dependent kinase 5 (CDK5) plays a biological role. In cases where the term is applied to a disease, state or condition, the biological role played by cyclin dependent kinase 5 (CDK5) may be direct or indirect and may be necessary and/or sufficient for the manifestation of the symptoms of the disease, state or condition (or its aetiology or progression). Thus, cyclin dependent kinase 5 (CDK5) activity (and in particular aberrant levels of cyclin dependent kinase 5 (CDK5) activity, e.g. cyclin dependent kinase 5 (CDK5) over-expression) need not necessarily be the proximal cause of the disease, state or condition: rather, it is contemplated that the CDK5-mediated diseases, states or conditions include those having multifactorial aetiologies and complex progressions in which CDK5. In cases where the term is applied to treatment, prophylaxis or intervention (e.g. in the “CDK5-mediated treatments” of the invention), the role played by CDK5 may be direct or indirect and may be necessary and/or sufficient for the operation of the treatment, prophylaxis or outcome of the intervention. Thus, a disease state or condition mediated by the cyclin dependent kinases 5(CDK) includes a disease state or condition which has arisen as a consequence of the development of resistance to any particular cancer drug or treatment (including in particular resistance to one or more of the compounds described herein).

The term “intervention” is a term of art used herein to define any agency which effects a physiological change at any level. Thus, the intervention may comprise the induction or repression of any physiological process, event, biochemical pathway or cellular/biochemical event. The interventions of the invention typically effect (or contribute to) the therapy, treatment or prophylaxis of a disease or condition.

As used herein, the term “pharmaceutical kit” defines an array of one or more unit doses of a pharmaceutical composition together with dosing means (e.g. measuring device) and/or delivery means (e.g. inhaler or syringe), optionally all contained within common outer packaging. In pharmaceutical kits comprising a combination of two or more compounds/agents, the individual compounds/agents may unitary or non-unitary formulations. The unit dose(s) may be contained within a blister pack. The pharmaceutical kit may optionally further comprise instructions for use.

As used herein, the term “pharmaceutical pack” defines an array of one or more unit doses of a pharmaceutical composition, optionally contained within common outer packaging. In pharmaceutical packs comprising a combination of two or more compounds/agents, the individual compounds/agents may unitary or non-unitary formulations. The unit dose(s) may be contained within a blister pack. The pharmaceutical pack may optionally further comprise instructions for use.

As used herein, the term “patient pack” defines a package, prescribed to a patient, which contains pharmaceutical compositions for the whole course of treatment. Patient packs usually contain one or more blister pack(s). Patient packs have an advantage over traditional prescriptions, where a pharmacist divides a patient's supply of a pharmaceutical from a bulk supply, in that the patient always has access to the package insert contained in the patient pack, normally missing in patient prescriptions. The inclusion of a package insert has been shown to improve patient compliance with the physician's instructions.

### General Preferences and Definitions for Compounds of Formula (0)

A wide variety of compounds of the formula (0) find application in the therapeutic uses upon which the present invention is based. The compounds of formula (0) for use in the treatment of pain or for the treatment of stroke correspond to those of formula (0) described in WO 2005/012256 (PCT/GB2004/003179), the contents of which are incorporated herein by reference, and include the various possible substituents, sub-groups, embodiments and examples thereof as therein defined. The content of WO 2005/012256 (PCT/GB2004/003179) describing the various possible substituents, subgroups, embodiments and examples of compounds of formula (0) are hereby incorporated herein by reference.

The formula (0) of WO 2005/012256 (PCT/GB2004/003179) is herein also referred to as formula (0) and references to formula (0) herein are to be interpreted accordingly.

Thus, the compound of formula (0) for use in the invention has the formula:

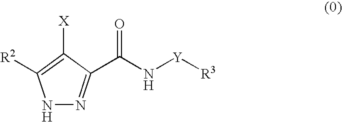

or salts or tautomers or N-oxides or solvates thereof;
wherein
X is a group R¹-A-NR⁴— or a 5- or 6-membered carbocyclic or heterocyclic ring;
A is a bond, SO₂, C═O, NR^{g}(C═O) or O(C═O) wherein R^{g} is hydrogen or C₁₋₄ hydrocarbyl optionally substituted by hydroxy or C₁₋₄ alkoxy;
Y is a bond or an alkylene chain of 1, 2 or 3 carbon atoms in length;
R¹ is hydrogen; a carbocyclic or heterocyclic group having from 3 to 12 ring members; or a C₁₋₈ hydrocarbyl group optionally substituted by one or more substituents selected from halogen (e.g. fluorine), hydroxy, C₁₋₄ hydrocarbyloxy, amino, mono- or di-C₁₋₄ hydrocarbylamino, and carbocyclic or heterocyclic groups having from 3 to 12 ring members, and wherein 1 or 2 of the carbon atoms of the hydrocarbyl group may optionally be replaced by an atom or group selected from O, S, NH, SO, SO₂;
R² is hydrogen; halogen; C₁₋₄ alkoxy (e.g. methoxy); or a C₁₋₄ hydrocarbyl group optionally substituted by halogen (e.g. fluorine), hydroxyl or C₁₋₄ alkoxy (e.g. methoxy);
R³ is selected from hydrogen and carbocyclic and heterocyclic groups having from 3 to 12 ring members; and
R⁴ is hydrogen or a C₁₋₄ hydrocarbyl group optionally substituted by halogen (e.g. fluorine), hydroxyl or C₁₋₄ alkoxy (e.g. methoxy),

Formula (0) as used herein includes the various possible substituents, subgroups, embodiments and examples thereof as defined in WO 2005/012256 (PCT/GB2004/003179), so that the general preferences and definitions defined in WO 2005/012256 (PCT/GB2004/003179) shall apply to each of the moieties X, Y, R^{g}, R¹ to R⁴ and any substituent, moieties, sub-definition, sub-group or embodiment thereof, unless the context indicates otherwise.

In particular the carbocyclic and heterocyclic groups forming part of X, R¹ and R³ may be optionally substituted as defined in WO 2005/012256.

Particular compounds of the formula (0) are those defined in, for example, the compounds of formulae (I⁰), (I), (Ia), (Ib), (II), (III), (IV), (IVa), (Va), (Vb), (VIa), (VIb), (VII) or (VIII), and any sub-groups thereof in PCT/GB2004/003179 (WO 2005/012256), the compounds listed in PCT/GB2004/003179 (WO 2005/012256) and the compounds exemplified in the Examples section of PCT/GB2004/003179 (WO 2005/012256), the aforementioned sections of PCT/GB2004/003179 (WO 2005/012256) being hereby incorporated by reference.

A preferred sub-group of CDK inhibitor compounds within WO 2005/012256 is represented by the formula (Va):

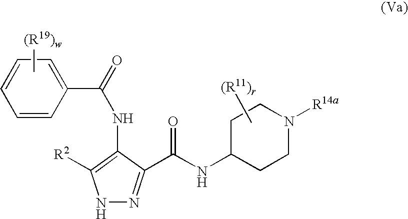

or salts or tautomers or N-oxides or solvates thereof;
wherein R^{14a} is selected from hydrogen, C₁₋₄ alkyl optionally substituted by fluoro (e.g. methyl, ethyl, n-propyl, i-propyl, butyl and 2,2,2-trifluoroethyl), cyclopropylmethyl, phenyl-C₁₋₂ alkyl (e.g. benzyl), C₁₋₄ alkoxycarbonyl (e.g. ethoxycarbonyl and t-butyloxycarbonyl), phenyl-C₁₋₂ alkoxycarbonyl (e.g. benzyloxycarbonyl), C₁₋₂-alkoxy-C₁₋₂ alkyl (e.g. methoxymethyl and methoxyethyl), and C₁₋₄ alkylsulphonyl (e.g. methanesulphonyl), wherein the phenyl moieties when present are optionally substituted by one to three substituents selected from fluorine, chlorine, C₁₋₄ alkoxy optionally substituted by fluoro or C₁₋₂-alkoxy, and C₁₋₄ alkyl optionally substituted by fluoro or C₁₋₂-alkoxy;
w is 0, 1, 2 or 3;
R² is hydrogen or methyl, most preferably hydrogen;
r is 0, 1 or 2;
R¹¹ is selected from hydrogen and C₁₋₃ alkyl (and more preferably is selected from hydrogen and methyl and most preferably is hydrogen); and
R¹⁹ is selected from fluorine; chlorine; C₁₋₄ alkoxy optionally substituted by fluoro or C₁₋₂-alkoxy; and C₁₋₄ alkyl optionally substituted by fluoro or C₁₋₂-alkoxy.

Particular compounds within formula (VIb) of WO 2005/012256 include:
4-(2,6-difluoro-benzoylamino)-1H-pyrazole-3-carboxylic acid piperidin-4-ylamide;
4-(2,6-difluoro-benzoylamino)-1H-pyrazole-3-carboxylic acid (1-methyl-piperidin-4-yl)-amide;
4-(2,6-dichloro-benzoylamino)-1H-pyrazole-3-carboxylic acid piperidin-4-ylamide; and
4-(2-fluoro-6-methoxy-benzoylamino)-1H-pyrazole-3-carboxylic acid piperidin-4-ylamide; or salts or tautomers or N-oxides or solvates thereof.

A preferred compound of the formula (0) is 4-(2,6-dichloro-benzoylamino)-1H-pyrazole-3-carboxylic acid piperidin-4-ylamide.

The compound 4-(2,6-dichloro-benzoylamino)-1H-pyrazole-3-carboxylic acid piperidin-4-ylamide may be present in the form of an acid addition salt which may be a salt formed with hydrochloric acid or a salt as described in WO 2006/077426, the contents of which are incorporated herein by reference. The salts may be prepared from 4-(2,6-dichloro-benzoylamino)-1H-pyrazole-3-carboxylic acid piperidin-4-ylamide by the methods described in WO 2006/077426.

In one preferred embodiment, the compound of formula (0) is 4-(2,6-dichloro-benzoylamino)-1H-pyrazole-3-carboxylic acid piperidin-4-ylamide in the form of a salt selected from the acid addition salts formed with hydrochloric acid, methanesulphonic acid and/or acetic acid.

One particular salt of 4-(2,6-dichloro-benzoylamino)-1H-pyrazole-3-carboxylic acid piperidin-4-ylamide is the methane sulphonic acid salt, and in particular the methane sulphonic acid salt of 4-(2,6-dichloro-benzoylamino)-1H-pyrazole-3-carboxylic acid piperidin-4-ylamide in a crystalline form.

In one embodiment, the salt is a methanesulphonic acid salt of 4-(2,6-dichlorobenzoylamino)-1H-pyrazole-3-carboxylic acid piperidin-4-ylamide mesylate salt which is crystalline and is characterised by any one or more (in any combination) or all of the following parameters, namely that the salt:

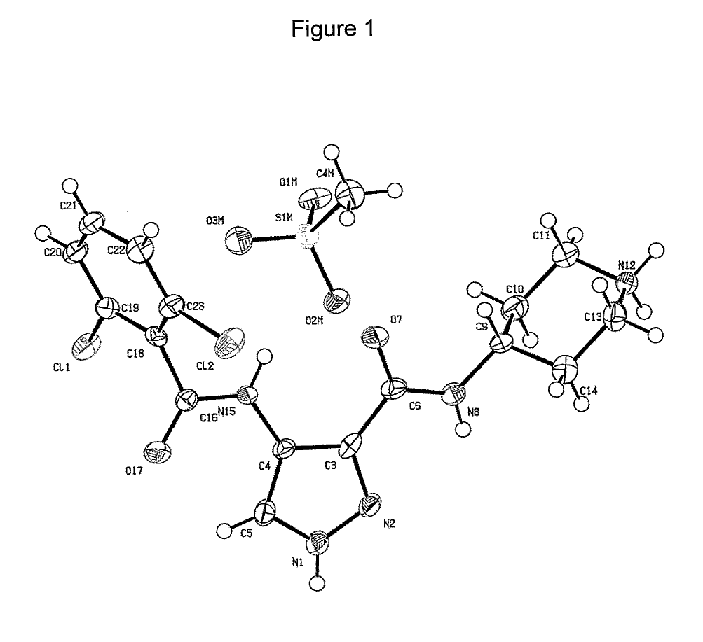

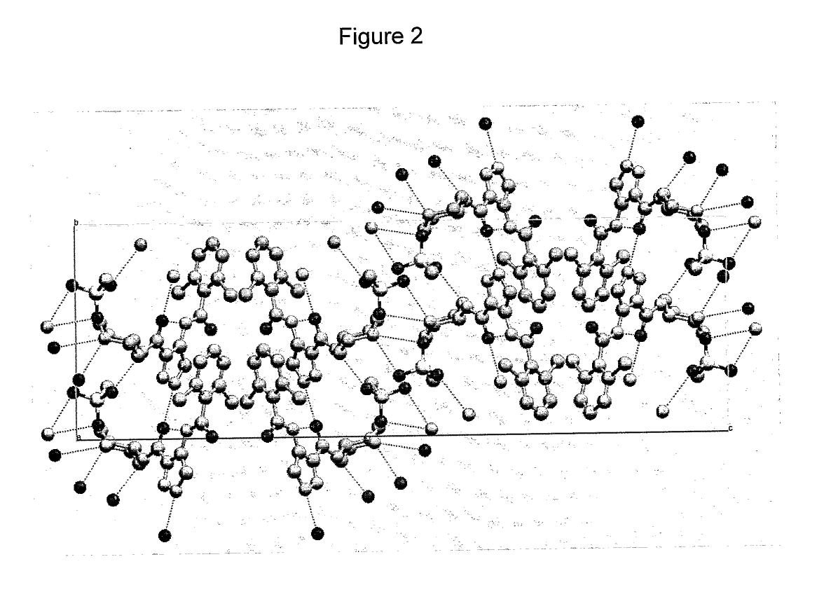

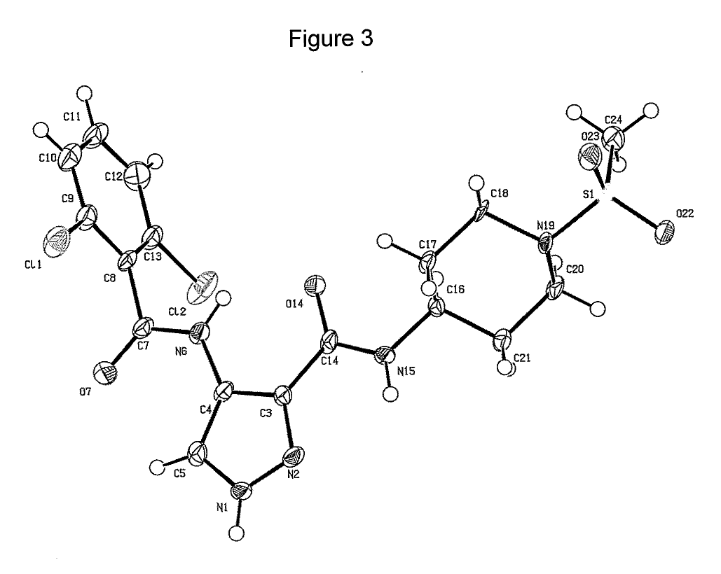

(a) has a crystal structure as set out in FIGS. 1 and 2 of WO 2006/077426; and/or
(b) has a crystal structure as defined by the coordinates in Example 2 of WO 2006/077426; and/or
(c) has crystal lattice parameters at 93 K a=8.90(10), b=12.44(10), c=38.49(4) Å, α=β=γ=90°; and/or
(d) has a crystal structure that belongs belong to an orthorhombic space group such as Pbca (# 61); and/or
(e) has an X-ray powder diffraction pattern characterised by the presence of major peaks at the diffraction angles (2θ) and interplanar spacings (d) set forth in Table A of WO 2006/077426, and optionally Table B of WO 2006/077426; for example wherein the X-ray powder diffraction pattern is characterised by the presence of major peaks at the diffraction angles (2θ), interplanar spacings (d) and intensities set forth in Table C of WO 2006/077426; and/or
(f) exhibits peaks at the same diffraction angles as those of the X-ray powder diffraction pattern shown in FIG. 3 of WO 2006/077426 and optionally wherein the peaks have the same relative intensity as the peaks in FIG. 3 of WO 2006/077426; and/or
(g) has an X-ray powder diffraction pattern substantially as shown in FIG. 3 of WO 2006/077426; and/or
(h) is anhydrous and exhibits an endothermic peak at 379-380° C. e.g. 379.8° C. when subjected to DSC; and/or
(i) exhibits an infra-red spectrum, when analysed using the KBr disc method, that contains characteristic peaks at 3233, 3002, 2829, 1679, 1632, 1560, 1430, 1198, 1037, 909 and 784 cm⁻¹.

Particular pharmaceutical compositions comprising an aqueous solution containing an acid addition salt of 4-(2,6-dichloro-benzoylamino)-1H-pyrazole-3-carboxylic acid piperidin-4-ylamide (such as the mesylate and acetate and mixtures thereof, and preferably the mesylate) are also described in WO 2006/077426.

Methods of Treatment using the compounds of Formula (0) are described in WO 2005/012256 pages 105 to 107, and WO 2006/077426 pages 58 to 61, and are further described herein. Methods of Diagnosis of a patient to determine whether a disease or condition from which the patient is or may be suffering is one which would be susceptible to treatment with a compound having activity against CDK are described in WO 2005/012256 pages 107 to 111, and WO 2006/077426 pages 62 to 65, and are further described herein.

Methods for the Preparation of Compounds of the Formula (0) are as described in WO 2005/012256, WO 2006/077416 and WO 2006/077426, the contents of which are incorporated herein by reference. In particular, the contents of WO 2005/012556 which relate to the relevant processes at pages 91 to 101 are hereby incorporated herein by reference. In particular, the contents of WO 2006/074416 which relate to the relevant processes at pages 33 to 39 are hereby incorporated herein by reference. In particular, the contents of WO 2006/077426 which relate to the relevant processes at pages 30 to 36 are hereby incorporated herein by reference.

### General Preferences and Definitions for Compounds of Formula (I′″)

A wide variety of compounds of the formula (I′″) find application in the combinations of the invention, as described in detail below. The compounds of formula (I′″) for use in the combinations of the invention correspond to those of formula (I) described in WO 2006/077416 and include the various possible substituents, sub-groups, embodiments and examples thereof as therein defined. The content of WO 2006/077416 describing the various possible substituents, subgroups, embodiments and examples of compounds of formula (I) (i.e. formula (I′″) herein) is hereby incorporated herein by reference. The formula (I) of WO 2006/077416 is herein referred to as formula (I′″) and references to formula (I′″) herein are to be interpreted accordingly.

In this specification in general (and this section in particular), unless the context indicates otherwise, references to a compound of formula (I′″) includes all subgroups of formula (I′″) as defined herein and the term ‘subgroups’ includes all preferences, embodiments, examples and particular compounds defined herein. Any references to formula (I′″) herein shall also be taken to refer to and any sub-group of compounds within formula (I′″) and any preferences and examples thereof unless the context requires otherwise.

Compounds of the formula (I′″) have the formula:

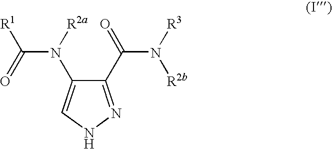

or salts, tautomers, solvates and N-oxides thereof;
wherein:
R¹ is 2,6-dichlorophenyl;
R^{2a} and R^{2b} are both hydrogen;
and R³ is a group:

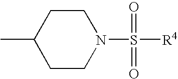

where R⁴ is C₁₋₄ alkyl.

Any references to formula (I′″) herein shall also be taken to refer to and any sub-group of compounds within formula (I′″) and any preferences and examples thereof unless the context requires otherwise. The following sections describe certain general preferences and definitions in relation to compounds of the formula (I′″) for use in the combinations of the invention.

The C₁₋₄ alkyl group can be a C₁, C₂, C₃ or C₄ alkyl group.

Within the group of C₁₋₄ alkyl groups are the sub-groups of:
C₁₋₃ alkyl groups;
C₁₋₂ alkyl groups;
C₂₋₃ alkyl groups; and
C₂₋₄ alkyl groups.

One particular sub-group is C₁₋₃ alkyl.

Particular C₁₋₄ alkyl groups are methyl, ethyl, i-propyl, n-butyl, i-butyl and tert-butyl groups.

Another sub-group of C₁₋₄ alkyl groups consists of methyl, ethyl, i-propyl and n-propyl groups.

One preferred group is a methyl group.

Other particular groups R⁴ are ethyl and isopropyl.

A preferred compound within formula (I′″) is 4-(2,6-dichloro-benzoylamino)-1H-pyrazole-3-carboxylic acid (1-methanesulphonyl-piperidin-4-yl)-amide.

In one embodiment, the 4-(2,6-dichloro-benzoylamino)-1H-pyrazole-3-carboxylic acid (1-methanesulphonyl-piperidin-4-yl)-amide is substantially crystalline or is a crystal form thereof.

The compound 4-(2,6-dichloro-benzoylamino)-1H-pyrazole-3-carboxylic acid (1-methanesulphonyl-piperidin-4-yl)-amide may be substantially crystalline; i.e. it is from 50% to 100% crystalline. Crystalline forms of 4-(2,6-dichloro-benzoylamino)-1H-pyrazole-3-carboxylic acid (1-methanesulphonyl-piperidin-4-yl)-amide are disclosed in our applications U.S. 60/746,541 and U.S. 60/830,967, the contents of each of which are incorporated herein by reference.

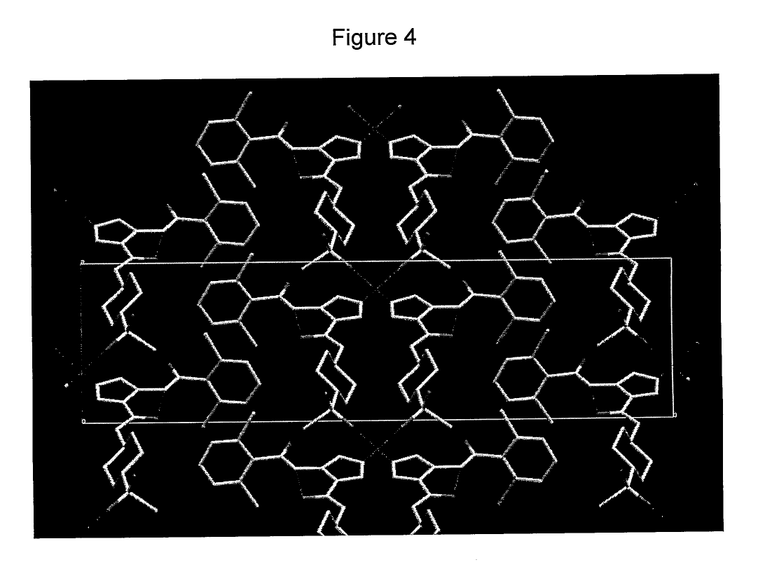

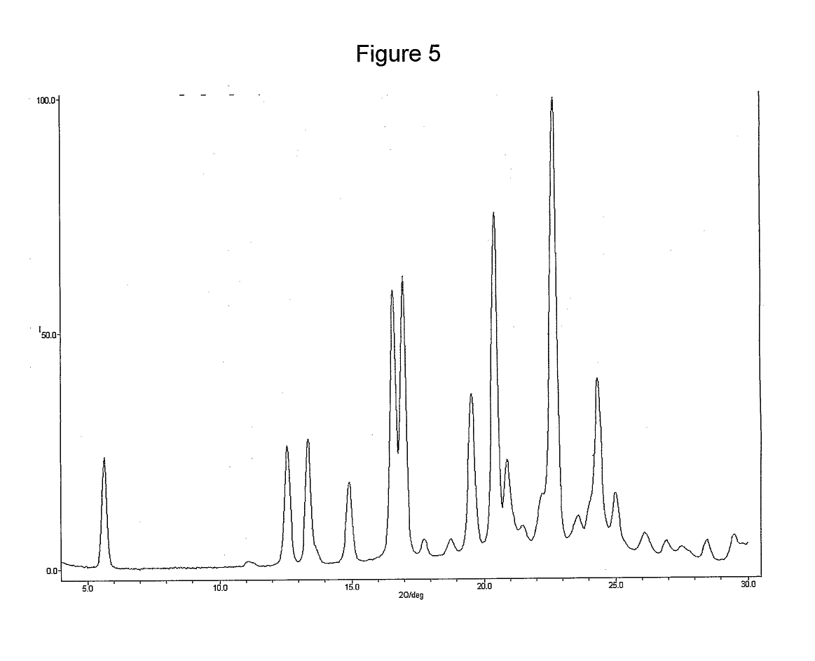

In one embodiment, the crystalline form of 4-(2,6-dichloro-benzoylamino)-1H-pyrazole-3-carboxylic acid (1-methanesulphonyl-piperidin-4-yl)-amide is a crystalline form which is characterised by any one or more (in any combination) or all of the following parameters, namely that the crystalline form:
has a crystal structure as set out in FIGS. 3 and 4 herein (and as set forth in U.S. 60/746,541 and U.S. 60/830,967); and/or
has a crystal structure as defined by the coordinates in Table 1 herein (and as set forth in U.S. 60/746,541 and U.S. 60/830,967); and/or
has crystal lattice parameters at a=9.15, b=31.32, c=7.93 Å, β=113.3°, α=γ=90°; and/or
has a crystal structure that belongs belong to a monoclinic space group such as C2/c (# 15); and/or
has an X-ray powder diffraction pattern characterised by the presence of major peaks at the diffraction angles (2θ) and interplanar spacings (d) set forth in Table A described herein; and optionally Table B.
   and/or
exhibits peaks at the same diffraction angles as those of the X-ray powder diffraction pattern shown in FIG. 5 herein (and as set out in FIG. 3 forth in U.S. 60/746,541 and U.S. 60/830,967) and optionally wherein the peaks have the same relative intensity as the peaks in FIG. 5 herein (and as set forth in U.S. 60/746,541 and U.S. 60/830,967); and/or
(has an X-ray powder diffraction pattern substantially as shown in FIG. 5 herein (and as set forth in U.S. 60/746,541 and U.S. 60/830,967); and/or
is anhydrous and exhibits an endothermic peak at an endothermic peak at 293-296° C., for example 294.5-295° C., when subjected to DSC; and/or
exhibits an infra-red spectrum, when analysed using the Universal Attenuated Total Reflectance (UATR) method, that contains characteristic peaks at containing characteristic peaks at 3362, 3019, 2843, 1677, 1577, 1547, 1533, 1326, 1150, 926, 781, 667 cm⁻¹.

Pharmaceutical compositions comprising a compound of formula (I′″) and a pharmaceutically acceptable carrier in a form suitable for oral administration are described in WO 2006/077416 at pages 37 to 48.

In particular a pharmaceutical composition comprising a substantially amorphous solid solution, said solid solution comprising (a) a compound of the formula (I′″), for example 4-(2,6-dichloro-benzoylamino)-1H-pyrazole-3-carboxylic acid (1-methanesulphonyl-piperidin-4-yl)-amide; and (b) a polymer selected from the group consisting of:

polyvinylpyrrolidone (povidone), crosslinked polyvinylpyrrolidone (crospovidone), hydroxypropyl methylcellulose, hydroxypropylcellulose, polyethylene oxide, gelatin, crosslinked polyacrylic acid (carbomer), carboxymethylcellulose, crosslinked carboxymethylcellulose (croscarmellose), methylcellulose, methacrylic acid copolymer, methacrylate copolymer, and water soluble salts such as sodium and ammonium salts of methacrylic acid and methacrylate copolymers, cellulose acetate phthalate, hydroxypropylmethylcellulose phthalate and propylene glycol alginate;
wherein the ratio of said compound to said polymer is about 1:1 to about 1:6, for example a 1:3 ratio, spray dried from a mixture of one of chloroform or dichloromethane and one of methanol or ethanol, preferably dichloromethane/ethanol in a 1:1 ratio.

Further compositions are disclosed in our applications U.S. 60/746,541 and U.S. 60/830,967, the contents of each of which are incorporated herein by reference.

Methods of Treatment using this compound are described in WO 2006/077416 at pages 48 to 52 and further described herein. Methods of Diagnosis of a patient to determine whether a disease or condition from which the patient is or may be suffering is one which would be susceptible to treatment with a compound having activity against CDK are described at pages 52 to 56 and further described herein.

Methods for the Preparation of Compounds of the Formula (I′″) are also as described in WO 2005/012256, WO 2006/077416 and WO 2006/077426, the contents of which are incorporated herein by reference. In particular, the contents of WO 2006/074416 which relate to the relevant processes at pages 33 to 39 are hereby incorporated herein by reference. Methods of Purification of the Compounds of Formula (I′″) are described at pages 36 to 37 of WO 2006/077416, which disclosure is hereby incorporated herein by reference.

### Salts, Solvates, Tautomers, Isomers, N-Oxides, Esters, Prodrugs and Isotopes of Compounds of Formula (I′″)

A reference to a particular compound of formulae (I′″) or subgroups or examples thereof includes ionic forms, salts, solvates, isomers, tautomers, N-oxides, esters, prodrugs, isotopes and protected forms thereof, for example, as discussed below; preferably, the salts or tautomers or isomers or N-oxides or solvates thereof; and more preferably, the salts or tautomers or N-oxides or solvates thereof.

Many compounds of the formulae (I′″) can exist in the form of salts, for example acid addition salts or, in certain cases salts of organic and inorganic bases such as carboxylate, sulphonate and phosphate salts. All such salts are within the scope of this invention, and references to compounds (e.g. to compounds of the formulae (0) or (I′″) include the salt forms of the compounds.

The salts can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two; generally, nonaqueous media such as ether, ethyl acetate, ethanol, isopropanol, or acetonitrile are used.

Acid addition salts may be formed with a wide variety of acids, both inorganic and organic. Examples of acid addition salts include salts formed with an acid selected from the group consisting of acetic, 2,2-dichloroacetic, adipic, alginic, ascorbic (e.g. L-ascorbic), L-aspartic, benzenesulphonic, benzoic, 4-acetamidobenzoic, butanoic, (+) camphoric, camphor-sulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrobromic, hydrochloric, hydriodic, isethionic, (+)-L-lactic, (±)-DL-lactic, lactobionic, maleic, malic, (−)-L-malic, malonic, (±)-DL-mandelic, methanesulphonic, naphthalene-2-sulphonic, naphthalene-1,5-disulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, (+)-L-tartaric, thiocyanic, p-toluenesulphonic, undecylenic and valeric acids, as well as acylated amino acids and cation exchange resins.

One particular group of salts consists of salts formed from acetic, hydrochloric, hydriodic, phosphoric, nitric, sulphuric, citric, lactic, succinic, maleic, malic, isethionic, fumaric, benzenesulphonic, toluenesulphonic, methanesulphonic (mesylate), ethanesulphonic, naphthalenesulphonic, valeric, acetic, propanoic, butanoic, malonic, glucuronic and lactobionic acids.

One sub-group of salts consists of salts formed from hydrochloric, acetic, methanesulphonic, adipic, L-aspartic and DL-lactic acids.

Another sub-group of salts consists of the acetate, mesylate, ethanesulphonate, DL-lactate, adipate, D-glucuronate, D-gluconate and hydrochloride salts.

Particular salts for use in the preparation of liquid (e.g. aqueous) compositions of the compounds of formulae (I′″) and sub-groups and examples thereof as described herein are salts having a solubility in a given liquid carrier (e.g. water) of greater than 10 μg/ml of the liquid carrier (e.g. water), more typically greater than 0.5 mg/ml and preferably greater than 1 mg/ml.

In one embodiment of the invention, there is provided a pharmaceutical composition comprising an aqueous solution containing a compound of the formula (I′″) and sub-groups and examples thereof as described herein in the form of a salt in a concentration of greater than greater than 10 μg/ml of the liquid carrier (e.g. water), more typically greater than 0.5 mg/ml and preferably greater than 1 mg/ml.

If the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth metal cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds contain an amine function, these may form quaternary ammonium salts, for example by reaction with an alkylating agent according to methods well known to the skilled person. Such quaternary ammonium compounds are within the scope of formula (I′″) as defined herein.

The salt forms of the compounds are typically pharmaceutically acceptable salts, and examples of pharmaceutically acceptable salts are discussed in Berge et al., 1977, “Pharmaceutically Acceptable Salts,” J. Pharm. Sci., Vol. 66, pp. 1-19. However, salts that are not pharmaceutically acceptable may also be prepared as intermediate forms which may then be converted into pharmaceutically acceptable salts. Such non-pharmaceutically acceptable salts forms, which may be useful, for example, in the purification or separation of the compounds, also form part of the invention.

Compounds of the formulae (I′″) containing an amine function may also form N-oxides. A reference herein to a compound of the formula (I′″) that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

Compounds of the formulae (I′″) may exist in a number of different geometric isomeric, and tautomeric forms and references to compounds of the formula (I′″) include all such forms. For the avoidance of doubt, where a compound can exist in one of several geometric isomeric or tautomeric forms and only one is specifically described or shown, all others are nevertheless contemplated (and are for example embraced by formula (0) or (I′″)).

For example, in compounds of the formula (I′″) the pyrazole ring can exist in the two tautomeric forms A and B below. For simplicity, the general formula (I′″) illustrates form A, but the formula is to be taken as embracing both tautomeric forms.

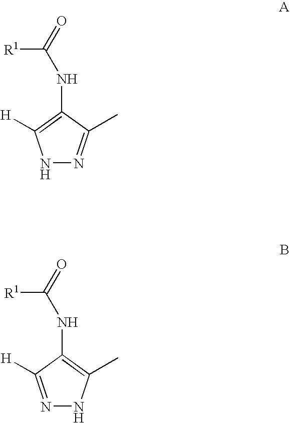

Similar considerations apply also to formula (0).

Other examples of tautomeric forms include, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

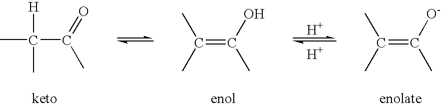

Where any compound of the formulae (I′″) contain one or more chiral centres (e.g. as in the case of the compounds wherein R⁴ is 2-butyl), and can exist in the form of two or more optical isomers, references to compounds of the formula (I′″) include all optical isomeric forms thereof (e.g. enantiomers, epimers and diastereoisomers), either as individual optical isomers, or mixtures (e.g. racemic mixtures) or two or more optical isomers, unless the context requires otherwise.

The optical isomers may be characterised and identified by their optical activity (i.e. as + and − isomers, or d and/isomers) or they may be characterised in terms of their absolute stereochemistry using the “R and S” nomenclature developed by Cahn, Ingold and Prelog, see Advanced Organic Chemistry by Jerry March, 4^{th} Edition, John Wiley & Sons, New York, 1992, pages 109-114, and see also Cahn, Ingold & Prelog, Angew. Chem. Int. Ed. Engl., 1966, 5, 385-415.

Optical isomers can be separated by a number of techniques including chiral chromatography (chromatography on a chiral support) and such techniques are well known to the person skilled in the art.

As an alternative to chiral chromatography, optical isomers can be separated by forming diastereoisomeric salts with chiral acids such as (+)-tartaric acid, (−)-pyroglutamic acid, (−)-di-toluoyl-L-tartaric acid, (+)-mandelic acid, (−)-malic acid, and (−)-camphorsulphonic, separating the diastereoisomers by preferential crystallisation, and then dissociating the salts to give the individual enantiomer of the free base.

Where compounds of the formulae (I′″) exist as two or more optical isomeric forms, one enantiomer in a pair of enantiomers may exhibit advantages over the other enantiomer, for example, in terms of biological activity. Thus, in certain circumstances, it may be desirable to use as a therapeutic agent only one of a pair of enantiomers, or only one of a plurality of diastereoisomers. Accordingly, the invention provides compositions containing a compound of the formula (I′″) having one or more chiral centres, wherein at least 55% (e.g. at least 60%, 65%, 70%, 75%, 80%, 85%, 90% or 95%) of the compound of the formula (I′″) is present as a single optical isomer (e.g. enantiomer or diastereoisomer). In one general embodiment, 99% or more (e.g. substantially all) of the total amount of the compound of the formula (I′″) may be present as a single optical isomer (e.g. enantiomer or diastereoisomer).

The compounds include compounds with one or more isotopic substitutions, and a reference to a particular element includes within its scope all isotopes of the element. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O.

The isotopes may be radioactive or non-radioactive. In one embodiment of the invention, the compounds contain no radioactive isotopes. Such compounds are preferred for therapeutic use. In another embodiment, however, the compound may contain one or more radioisotopes. Compounds containing such radioisotopes may be useful in a diagnostic context.

Esters such as carboxylic acid esters and acyloxy esters of the compounds of formulae (I′″) bearing a carboxylic acid group or a hydroxyl group are also contemplated and are embraced by formulae (I′″). Examples of esters are compounds containing the group —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Particular examples of ester groups include, but are not limited to, —C(═O)OCH₃, —C(═O)OCH₂CH₃, —C(═O)OC(CH₃)₃, and —C(═O)OPh. Examples of acyloxy (reverse ester) groups are represented by —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Particular examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Also encompassed by formulae (I′″) are any polymorphic forms of the compounds, solvates (e.g. hydrates), complexes (e.g. inclusion complexes or clathrates with compounds such as cyclodextrins, or complexes with metals) of the compounds, and pro-drugs of the compounds of formulae (I′″). By “prodrugs” is meant for example any compound that is converted in vivo into a biologically active compound (e.g. into a compound of the formula (I′″)).

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Examples of such metabolically labile esters include those of the formula —C(═O)OR wherein R is:
C₁₋₇alkyl
(e.g., -Me, -Et, -nPr, -iPr, -nBu, -sBu, -iBu, -tBu);
C₁₋₇-aminoalkyl
(e.g., aminoethyl; 2-(N,N-diethylamino)ethyl; 2-(4-morpholino)ethyl); and
acyloxy-C₁₋₇alkyl
(e.g., acyloxymethyl;
acyloxyethyl;
pivaloyloxymethyl;
acetoxymethyl;
1-acetoxyethyl;
1-(1-methoxy-1-methyl)ethyl-carbonxyloxyethyl;
1-(benzoyloxy)ethyl; isopropoxy-carbonyloxymethyl;
1-isopropoxy-carbonyloxyethyl; cyclohexyl-carbonyloxymethyl;
1-cyclohexyl-carbonyloxyethyl;
cyclohexyloxy-carbonyloxymethyl;
1-cyclohexyloxy-carbonyloxyethyl;
(4-tetrahydropyranyloxy) carbonyloxymethyl;
1-(4-tetrahydropyranyloxy)carbonyloxyethyl;
(4-tetrahydropyranyl)carbonyloxymethyl; and
1-(4-tetrahydropyranyl)carbonyloxyethyl).

Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

### Salts, Solvates, Tautomers, Isomers, N-Oxides, Esters, Prodrugs and Isotopes of Compounds of Formula (0)

Salts, Solvates, Tautomers, Isomers, N-Oxides, Esters, Prodrugs and Isotopes of compounds of Formula (0) and subgroups thereof are as defined in WO 2005/012256 at pages 81-88.

References to the compound 4-(2,6-dichloro-benzoylamino)-1H-pyrazole-3-carboxylic acid piperidin-4-ylamide and its acid addition salts include within their scope all solvates, tautomers and isotopes thereof and, where the context admits, N-oxides, other ionic forms and prodrugs.

The acid addition salt may be selected from salts formed with an acid selected from the group consisting of acetic, adipic, alginic, ascorbic (e.g. L-ascorbic), aspartic (e.g. L-aspartic), benzenesulphonic, benzoic, camphoric (e.g. (+) camphoric), capric, caprylic, carbonic, citric, cyclamic, dodecanoate, dodecylsulphuric, ethane-1,2-disulphonic, ethanesulphonic, fumaric, galactaric, gentisic, glucoheptonic, D-gluconic, glucuronic (e.g. D-glucuronic), glutamic (e.g. L-glutamic), α-oxoglutaric, glycolic, hippuric, isethionic, isobutyric, lactic (e.g. (+)-L-lactic and (±)-DL-lactic), lactobionic, laurylsulphonic, maleic, malic, (−)-L-malic, malonic, methanesulphonic, mucic, naphthalenesulphonic (e.g. naphthalene-2-sulphonic), naphthalene-1,5-disulphonic, nicotinic, oleic, orotic, oxalic, palmitic, pamoic, phosphoric, propionic, sebacic, stearic, succinic, sulphuric, tartaric (e.g. (+)-L-tartaric), thiocyanic, toluenesulphonic (e.g. p-toluenesulphonic), valeric and xinafoic acids.

One sub-group of acid addition salts includes salts formed with an acid selected from the group consisting of acetic, adipic, ascorbic (e.g. L-ascorbic), aspartic (e.g. L-aspartic), caproic, carbonic, citric, dodecanoic, fumaric, galactaric, glucoheptonic, gluconic (e.g. D-gluconic), glucuronic (e.g. D-glucuronic), glutamic (e.g. L-glutamic), glycolic, hippuric, lactic (e.g. (+)-L-lactic and (±)-DL-lactic), maleic, palmitic, phosphoric, sebacic, stearic, succinic, sulphuric, tartaric (e.g. (+)-L-tartaric) and thiocyanic acids.

More particularly the salts are acid addition salts formed with an acid selected from methanesulphonic acid and acetic acid, and mixtures thereof.

In one embodiment, the salt is an acid addition salt formed with methanesulphonic acid.

In another embodiment, the salt is an acid addition salt formed with acetic acid.

For convenience the salts formed from methanesulphonic acid and acetic acid may be referred to herein as the methanesulphonate or mesylate salts and acetate salts respectively.

In the solid state, the salts can be crystalline or amorphous or a mixture thereof.

In one embodiment, the salts are amorphous.

In an amorphous solid, the three dimensional structure that normally exists in a crystalline form does not exist and the positions of the molecules relative to one another in the amorphous form are essentially random, see for example Hancock et al. J. Pharm. Sci. (1997), 86, 1).

In another embodiment, the salts are substantially crystalline; i.e. they are from 50% to 100% crystalline, and more particularly they may be at least 50% crystalline, or at least 60% crystalline, or at least 70% crystalline, or at least 80% crystalline, or at least 90% crystalline, or at least 95% crystalline, or at least 98% crystalline, or at least 99% crystalline, or at least 99.5% crystalline, or at least 99.9% crystalline, for example 100% crystalline.

In a further embodiment, the salts are selected from the group consisting of salts that are from 50% to 100% crystalline, salts that are at least 50% crystalline, salts that are at least 60% crystalline, salts that are at least 70% crystalline, salts that are at least 80% crystalline, salts that are at least 90% crystalline, salts that are at least 95% crystalline, salts that are at least 98% crystalline, salts that are at least 99% crystalline, salts that are at least 99.5% crystalline, and salts that are at least 99.9% crystalline, for example 100% crystalline.

More preferably the salts may be those (or may be selected from the group consisting of those) that are 95% to 100% crystalline, for example at least 98% crystalline, or at least 99% crystalline, or at least 99.5% crystalline, or at least 99.6% crystalline or at least 99.7% crystalline or at least 99.8% crystalline or at least 99.9% crystalline, for example 100% crystalline.

One example of a substantially crystalline salt is a crystalline salt formed with methanesulphonic acid.

Another example of a substantially crystalline salt is a crystalline salt formed with acetic acid.

The salts, in the solid state, can be solvated (e.g. hydrated) or non-solvated (e.g. anhydrous).

In one embodiment, the salts are non-solvated (e.g. anhydrous). An example of a non-solvated salt is the crystalline salt formed with methanesulphonic acid as defined herein.

The term “anhydrous” as used herein does not exclude the possibility of the presence of some water on or in the salt (e.g a crystal of the salt). For example, there may be some water present on the surface of the salt (e.g. salt crystal), or minor amounts within the body of the salt (e.g. crystal). Typically, an anhydrous form contains fewer than 0.4 molecules of water per molecule of compound, and more preferably contains fewer than 0.1 molecules of water per molecule of compound, for example 0 molecules of water.

In another embodiment, the salts are solvated. Where the salts are hydrated, they can contain, for example, up to three molecules of water of crystallisation, more usually up to two molecules of water, e.g. one molecule of water or two molecules of water. Non-stoichiometric hydrates may also be formed in which the number of molecules of water present is less than one or is otherwise a non-integer. For example, where there is less than one molecule of water present, there may be for example 0.4, or 0.5, or 0.6, or 0.7, or 0.8, or 0.9 molecules of water present per molecule of compound.

Other solvates include alcoholates such as ethanolates and isopropanolates.

The salts can be synthesized from the parent compound 4-(2,6-dichloro-benzoylamino)-1H-pyrazole-3-carboxylic acid piperidin-4-ylamide by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the parent compound 4-(2,6-dichloro-benzoylamino)-1H-pyrazole-3-carboxylic acid piperidin-4-ylamide with the appropriate acid in water or in an organic solvent, or in a mixture of the two; generally, nonaqueous media such as ether, ethyl acetate, ethanol, isopropanol, or acetonitrile are used.

One method of preparing an acid addition salt of 4-(2,6-dichloro-benzoylamino)-1H-pyrazole-3-carboxylic acid piperidin-4-ylamide, which method comprises forming a solution of 4-(2,6-dichloro-benzoylamino)-1H-pyrazole-3-carboxylic acid piperidin-4-ylamide free base in a solvent (typically an organic solvent) or mixture of solvents, and treating the solution with an acid to form a precipitate of the acid addition salt.

The acid may be added as a solution in a solvent which is miscible with the solvent in which the free base is dissolved. The solvent in which the free base is initially dissolved may be one in which the acid addition salt thereof is insoluble. Alternatively, the solvent in which the free base is initially dissolved may be one in which the acid addition salt is at least partially soluble, a different solvent in which the acid addition salt is less soluble subsequently being added such that the salt precipitates out of solution.

In an alternative method of forming an acid addition salt, 4-(2,6-dichloro-benzoylamino)-1H-pyrazole-3-carboxylic acid piperidin-4-ylamide is dissolved in a solvent comprising a volatile acid and optionally a co-solvent, thereby to form a solution of the acid addition salt with the volatile acid, and the resulting solution is then concentrated or evaporated to isolate the salt. An example of an acid addition salt that can be made in this way is the acetate salt.

In another aspect, the invention provides an acid addition salt of 4-(2,6-dichloro-benzoylamino)-1H-pyrazole-3-carboxylic acid piperidin-4-ylamide as defined herein for use in the treatment of a CDK5 mediated disease as defined herein in particular stroke or pain, wherein the acid addition salt has been formed by a method comprising treating a compound of the formula (X):

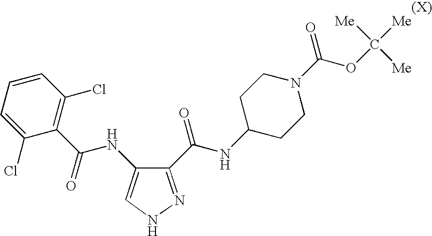

with an organic or inorganic acid as defined herein, other than hydrochloric acid, in an organic solvent to remove the tert-butyloxycarbonyl group and form an acid addition salt of 4-(2,6-dichloro-benzoylamino)-1H-pyrazole-3-carboxylic acid piperidin-4-ylamide with the organic or inorganic acid, and isolating the acid addition salt thus formed.

The salt is typically precipitated from the organic solvent as it is formed and hence can be isolated by separation of the solid from the solution, e.g. by filtration.

One salt form can be converted to the free base and optionally to another salt form by methods well known to the skilled person. For example, the free base can be formed by passing the salt solution through a column containing an amine stationary phase (e.g. a Strata-NH₂ column). Alternatively, a solution of the salt in water can be treated with sodium bicarbonate to decompose the salt and precipitate out the free base. The free base may then be combined with another acid by one of the methods described above or elsewhere herein.

The methanesulphonate salt form is particularly advantageous because of its good stability at elevated temperatures and in conditions of high relative humidity, its non-hygroscopicity (as defined herein), absence of polymorph and hydrate formation, and stability in aqueous conditions. Moreover, it has excellent water solubility and has better physiochemical properties (such as a high melting point) relative to other salts.

The term ‘stable’ or ‘stability’ as used herein includes chemical stability and solid state (physical) stability. The term ‘chemical stability’ means that the compound can be stored in an isolated form, or in the form of a formulation in which it is provided in admixture with for example, pharmaceutically acceptable carriers, diluents or adjuvants as described herein, under normal storage conditions, with little or no chemical degradation or decomposition. ‘Solid-state stability’ means the compound can be stored in an isolated solid form, or the form of a solid formulation in which it is provided in admixture with, for example, pharmaceutically acceptable carriers, diluents or adjuvants as described herein, under normal storage conditions, with little or no solid-state transformation (e.g. hydration, dehydration, solvatisation, desolvatisation, crystallisation, recrystallisation or solid-state phase transition).

The terms “non-hygroscopic” and “non-hygroscopicity” and related terms as used herein refer to substances that absorb less than 5% by weight (relative to their own weight) of water when exposed to conditions of high relative humidity, for example 90% relative humidity, and/or do not undergo changes in crystalline form in conditions of high humidity and/or do not absorb water into the body of the crystal (internal water) in conditions of high relative humidity.

Preferred salts for use in the preparation of liquid (e.g. aqueous) pharmaceutical compositions are acid addition salts (such as the mesylate and acetate and mixtures thereof as defined herein) having a solubility in a given liquid carrier (e.g. water) of greater than 15 mg/ml of the liquid carrier (e.g. water), more typically greater than 20 mg/ml, preferably greater than 25 mg/ml, and more preferably greater than 30 mg/ml.

In another aspect, there is provided a pharmaceutical composition comprising an aqueous solution containing an acid addition salt of 4-(2,6-dichloro-benzoylamino)-1H-pyrazole-3-carboxylic acid piperidin-4-ylamide (such as the mesylate and acetate and mixtures thereof as defined herein, and preferably the mesylate) in a concentration of greater than 15 mg/ml, typically greater than 20 mg/ml, preferably greater than 25 mg/ml, and more preferably greater than 30 mg/ml, for use in the treatment of stroke or pain.

In a preferred embodiment, the pharmaceutical composition comprises an aqueous solution containing an acid addition salt of 4-(2,6-dichloro-benzoylamino)-1H-pyrazole-3-carboxylic acid piperidin-4-ylamide selected from an acetate or methanesulphonate salt or a mixture thereof in a concentration of greater than 15 mg/ml, typically greater than 20 mg/ml, preferably greater than 25 mg/ml, and more preferably greater than 30 mg/ml.

In another aspect, the invention provides an aqueous solution of an acid addition salt of 4-(2,6-dichloro-benzoylamino)-1H-pyrazole-3-carboxylic acid piperidin-4-ylamide (such as the mesylate and acetate and mixtures thereof as defined herein), wherein the aqueous solution has a pH of 2 to 12, for example 2 to 9, and more particularly 4 to 7, for use in the treatment of stroke or pain.

In the aqueous solutions defined above, the acid addition salt may be any of the salts described herein but, in one preferred embodiment, is a mesylate or acetate salt as defined herein, and in particular the mesylate salt.

The invention also provides an aqueous solution of 4-(2,6-dichloro-benzoylamino)-1H-pyrazole-3-carboxylic acid piperidin-4-ylamide in protonated form together with one or more counter ions and optionally one or more further counter ions. In one embodiment one of the counter ions is selected from methanesulphonate and acetate. In another embodiment one of the counter ions is from the formulation buffer as described herein such as acetate. In a further embodiment there may be one or more further counter ions such as a chloride ion (e.g. from saline), for use in the treatment of stroke or pain.

The invention therefore provides an aqueous solution of 4-(2,6-dichloro-benzoylamino)-1H-pyrazole-3-carboxylic acid piperidin-4-ylamide in protonated form together with one or more counter ions selected from methanesulphonate and acetate and optionally one or more further counter ions such as a chloride ion, for use in the treatment of stroke or pain.

In the situation where there is more than one counter ion the aqueous solution of 4-(2,6-dichloro-benzoylamino)-1H-pyrazole-3-carboxylic acid piperidin-4-ylamide in protonated form will potentially contain a mixture of counter ions for example a mixture of methanesulphonate and acetate counter ions and optionally one or more further counter ions such as a chloride ion.

The invention therefore provides an aqueous solution of 4-(2,6-dichloro-benzoylamino)-1H-pyrazole-3-carboxylic acid piperidin-4-ylamide in protonated form together with one or more counter ions selected from methanesulphonate and acetate and optionally one or more further counter ions such as a chloride ion, and a mixture thereof, for use in the treatment of stroke or pain.

The aqueous solutions can be formed inter alia by dissolving a mesylate salt in a solution of acetate ions (e.g an acetate buffer) or by dissolving an acetate salt in a solution of mesylate ions. The mesylate and acetate ions may be present in the solution in a mesylate:acetate ratio of from 10:1 or less, for example 10:1 to 1:10, more preferably less then 8:1, or less than 7:1, or less than 6:1, or less than 5:1 or less than 4:1 or less than 3:1 or less than 2:1 or less than 1:1, more particularly from 1:1 to 1:10. In one embodiment, the mesylate and acetate ions are present in the solution in a mesylate:acetate ratio of from 1:1 to 1:10, for example 1:1 to 1:8, or 1:1 to 1:7 or 1:1 to 1:6 or 1:1 to 1:5, e.g. approximately 1:4.8.

The aqueous solutions of the salts may be buffered or unbuffered but in one embodiment are buffered.

In the context of the acid addition salt formed with methanesulphonic acid, a preferred buffer is a buffer formed from acetic acid and sodium acetate, for example at a solution pH of approximately 4.6. At this pH and in the acetate buffer, the methanesulphonic acid salt has a solubility of about 35 mg/ml.

The salts of the invention are typically pharmaceutically acceptable salts, and examples of pharmaceutically acceptable salts are discussed in Berge et al., 1977, “Pharmaceutically Acceptable Salts,” J. Pharm. Sci., Vol. 66, pp. 1-19. However, salts that are not pharmaceutically acceptable may also be prepared as intermediate forms which may then be converted into pharmaceutically acceptable salts. Such non-pharmaceutically acceptable salt forms therefore also form part of the invention.

### Crystalline Forms of the Compounds of Formula (I′″)

4-(2,6-dichloro-benzoylamino)-1H-pyrazole-3-carboxylic acid (1-methanesulphonyl-piperidin-4-yl)-amide for use in the therapeutic methods of the invention can be in a substantially crystalline form.

Although 4-(2,6-dichloro-benzoylamino)-1H-pyrazole-3-carboxylic acid (1-methanesulphonyl-piperidin-4-yl)-amide can form salts with the basic nitrogen atom in the pyrazole ring, references to the compound in substantially crystalline form are references to the free base.

References to the compound 4-(2,6-dichloro-benzoylamino)-1H-pyrazole-3-carboxylic acid (1-methanesulphonyl-piperidin-4-yl)-amide, where the context admits, include within their scope all solvates, tautomers and isotopes thereof.

Where the 4-(2,6-dichloro-benzoylamino)-1H-pyrazole-3-carboxylic acid (1-methanesulphonyl-piperidin-4-yl)-amide is substantially crystalline, it is from 50% to 100% crystalline.

More particularly, 4-(2,6-dichloro-benzoylamino)-1H-pyrazole-3-carboxylic acid (1-methanesulphonyl-piperidin-4-yl)-amide may be at least 55% crystalline, or at least 60% crystalline, or at least 65% crystalline, or at least 70% crystalline, or at least 75% crystalline, or at least 80% crystalline, or at least 85% crystalline, or at least 90% crystalline, or at least 95% crystalline, or at least 98% crystalline, or at least 99% crystalline, or at least 99.5% crystalline, or at least 99.9% crystalline, for example 100% crystalline.

The crystalline forms of 4-(2,6-dichloro-benzoylamino)-1H-pyrazole-3-carboxylic acid (1-methanesulphonyl-piperidin-4-yl)-amide may be solvated (e.g. hydrated) or non-solvated (e.g. anhydrous).

The term “anhydrous” as used herein does not exclude the possibility of the presence of some water on or in the compound (e.g. a crystal of the compound). For example, there may be some water present on the surface of the compound (e.g. compound crystal), or minor amounts within the body of the compound (e.g. crystal). Typically, an anhydrous form contains fewer than 0.4 molecules of water per molecule of compound, and more preferably contains fewer than 0.1 molecules of water per molecule of compound, for example 0 molecules of water.

In one embodiment, the therapeutic uses of the invention employ anhydrous 4-(2,6-dichloro-benzoylamino)-1H-pyrazole-3-carboxylic acid (1-methanesulphonyl-piperidin-4-yl)-amide.

In another embodiment, the therapeutic uses of the invention employ 4-(2,6-dichloro-benzoylamino)-1H-pyrazole-3-carboxylic acid (1-methanesulphonyl-piperidin-4-yl)-amide in a solvated, e.g. hydrated, form. Where the compound is hydrated, it can contain, for example, up to three molecules of water of crystallisation, more usually up to two molecules of water, e.g. one molecule of water or two molecules of water. Non-stoichiometric hydrates may also be formed in which the number of molecules of water present is less than one or is otherwise a non-integer. For example, where there is less than one molecule of water present, there may be for example 0.4, or 0.5, or 0.6, or 0.7, or 0.8, or 0.9 molecules of water present per molecule of compound.

Other solvates include alcoholates such as ethanolates and isopropanolates.

The crystals of 4-(2,6-dichloro-benzoylamino)-1H-pyrazole-3-carboxylic acid (1-methanesulphonyl-piperidin-4-yl)-amide and their crystal structure can be characterised using a number of techniques including single crystal X-ray crystallography, X-ray powder diffraction (XRPD), differential scanning calorimetry (DSC) and infra red spectroscopy, e.g. Fourier Transform infra-red spectroscopy (FTIR). The behaviour of the crystals under conditions of varying humidity can be analysed by gravimetric vapour sorption studies and also by XRPD.

Determination of the crystal structure of a compound can be performed by X-ray crystallography which can be carried out according to conventional methods, such as those described herein and in Fundamentals of Crystallography, C. Giacovazzo, H. L. Monaco, D. Viterbo, F. Scordari, G. Gilli, G. Zanotti and M. Catti, (International Union of Crystallography/Oxford University Press, 1992 ISBN 0-19-855578-4 (p/b), 0-19-85579-2 (h/b)). This technique involves the analysis and interpretation of the X-ray diffraction of a single crystal.

In the substantially crystalline form of 4-(2,6-dichloro-benzoylamino)-1H-pyrazole-3-carboxylic acid (1-methanesulphonyl-piperidin-4-yl)-amide, one single crystalline form may predominate, although other crystalline forms may be present in minor and preferably negligible amounts.

In one preferred embodiment, the invention provides a therapeutic use as defined herein wherein the compound is substantially crystalline 4-(2,6-dichloro-benzoylamino)-1H-pyrazole-3-carboxylic acid (1-methanesulphonyl-piperidin-4-yl)-amide containing a single crystalline form of the dehydrate of the compound and no more than 5% by weight of any other crystalline forms of the compound.

Preferably, the single crystalline form is accompanied by less than 4%, or less than 3%, or less than 2% of other crystalline forms, and in particular contains less than or equal to about 1% by weight of other crystalline forms. More preferably, the single crystalline form is accompanied by less than 0.9%, or less than 0.8%, or less than 0.7%, or less than 0.6%, or less than 0.5%, or less than 0.4%, or less than 0.3%, or less than 0.2%, or less than 0.1%, or less than 0.05%, or less than 0.01%, by weight of other crystalline forms, for example 0% by weight of other crystalline forms.

The crystalline forms of 4-(2,6-dichloro-benzoylamino)-1H-pyrazole-3-carboxylic acid (1-methanesulphonyl-piperidin-4-yl)-amide can be prepared by synthesizing the compound using the methods described in PCT/GB2006/000193 or methods described herein, and then subjecting the compound to one or more recrystallisation steps.

The use of the term “recrystallisation” herein does not require the compound to be in a crystalline form before the recrystallisation process. On the contrary, although the starting material for the recrystallisation process can be crystalline or partly crystalline, it may alternatively be in an amorphous form prior to recrystallisation.

The recrystallisation of 4-(2,6-dichloro-benzoylamino)-1H-pyrazole-3-carboxylic acid (1-methanesulphonyl-piperidin-4-yl)-amide can be carried out by methods well known to the skilled person. As is well known, a good recrystallization solvent should dissolve a moderate quantity of the substance to be purified at elevated temperatures but only a small quantity of the substance at lower temperature. It should dissolve impurities readily at low temperatures or not at all. Finally, the solvent should be readily removed from the purified product. This usually means that it has a relatively low boiling point and a person skilled in the art will know recrystallizing solvents for a particular substance or, if that information is not available, will test several solvents until an appropriate solvent or solvent mixture is found. In order to get a good yield of purified material, the minimum amount of hot solvent to dissolve all the impure material is used. In practice, 3-5% more solvent than necessary typically is used so that the solution is not saturated. If the impure compound contains an impurity which is insoluble in the solvent it may then be removed by filtration and then allowing the solution to crystallize. In addition, if the impure compound contains traces of coloured material that are not native to the compound, they may be removed by adding a small amount of decolorizing charcoal to the hot solution, filtering it and then allowing it to crystallize. Crystallization may occur spontaneously upon cooling the solution. However, if it does not occur spontaneously, then crystallization may be induced by cooling the solution below room temperature or by adding a single crystal of pure material (a seed crystal). Recrystallisation can also be carried out and/or the yield optimized by the use of an anti-solvent. In this case, the compound is dissolved in a suitable solvent at elevated temperature, filtered and then an additional solvent in which the required compound has low solubility is added to aid crystallization. The crystals are then typically isolated using vacuum filtration, washed and then dried, for example, in an oven or via desiccation.

Other examples of methods for crystallization include crystallization from a vapour, which includes an evaporation step, for example in a sealed tube or an air stream, and crystallization from melt (Crystallization Technology Handbook 2nd Edition, edited by A. Mersmann, 2001).

In one particular embodiment, thecrystalline form of 4-(2,6-dichloro-benzoylamino)-1H-pyrazole-3-carboxylic acid (1-methanesulphonyl-piperidin-4-yl)-amide, the crystalline form of 4-(2,6-dichloro-benzoylamino)-1H-pyrazole-3-carboxylic acid (1-methanesulphonyl-piperidin-4-yl)-amide is prepared by recrystallising the compound using a mixture of N,N-dimethylacetamide, acetone and water.

For example, the 4-(2,6-dichloro-benzoylamino)-1H-pyrazole-3-carboxylic acid (1-methanesulphonyl-piperidin-4-yl)-amide can be recrystallised by a method involving the steps of:

(a) dissolving the compound in a mixture of N,N-dimethylacetamide and acetone (e.g. in a volume ratio of 1.5:2) with heating (e.g. to a temperature of up to about 50° C., for example 40 to 50° C.);
(b) optionally clarifying the solution where required by filtration;
(c) adding water whilst maintaining or increasing the heating (e.g. to a temperature of 60 to 80° C.);
(d) cooling the solution, or allowing the solution to cool, to enable crystallisation to take place; and
(e) isolating the crystalline form of the compound, for example by filtration.

Crystals of 4-(2,6-dichloro-benzoylamino)-1H-pyrazole-3-carboxylic acid (1-methanesulphonyl-piperidin-4-yl)-amide prepared using the N,N-dimethylacetamide/acetone/water solvent system have been subjected to characterisation by X-ray crystallography.

Table 1 gives coordinate data for crystals of 4-(2,6-dichloro-benzoylamino)-1H-pyrazole-3-carboxylic acid (1-methanesulphonyl-piperidin-4-yl)-amide in Crystallographic Information File (CIF) Format (see Hall, Allen and Brown, Acta Cryst. (1991). A47, 655-685; http://www.iucr.ac.uk/iucr-top/cif/home.html). Alternative file formats such as a PDB file format (e.g. format consistent with that of the EBI Macromolecular Structure Database (Hinxton, UK)) may be used or preferred by others of skill in the art. However it will be apparent that the use of a different file format to present or manipulate the coordinates of the Tables is within the scope of the present invention. The crystal structure of the compound is illustrated in FIGS. 3 and 4, the thermal ellipsoid representation of the structure generated by the X-ray diffraction study being provided in FIG. 3 and the packing diagram being provided in FIG. 4.

From the X-ray crystallography studies, it has been found that the compound of the invention has a crystal structure that belongs belong to a monoclinic space group such as C2/c (# 15) with crystal lattice parameters a=9.15, b=31.32, c=7.93 Å, β=113.3°, α=γ=90°.

Accordingly, in another embodiment, the invention provides, for any of the therapeutic uses of the invention, a crystalline form of 4-(2,6-dichloro-benzoylamino)-1H-pyrazole-3-carboxylic acid (1-methanesulphonyl-piperidin-4-yl)-amide which:

(a) has a crystal structure as set out in FIGS. 3 and 4; and/or
(b) has a crystal structure as defined by the coordinates in Table 1 herein; and/or
(c) has crystal lattice parameters at a=9.15, b=31.32, c=7.93 Å, β=113.3°, α=γ=90°; and/or
(d) has a crystal structure that belongs belong to a monoclinic space group such as C2/c (# 15).

Alternatively, or additionally, the crystalline structure of the crystalline compound of the invention can be analysed by the solid state technique of X-ray Powder Diffraction (XRPD). XRPD can be carried out according to conventional methods such as those described herein (see the examples) and in Introduction to X-ray Powder Diffraction, Ron Jenkins and Robert L. Snyder (John Wiley & Sons, New York, 1996). The presence of defined peaks (as opposed to random background noise) in an XRPD diffractogram indicates that the compound has a degree of crystallinity.

A compound's X-ray powder pattern is characterised by the diffraction angle (2θ) and interplanar spacing (d) parameters of an X-ray diffraction spectrum. These are related by Bragg's equation, nλ=2d Sin θ, (where n=1; λ=wavelength of the cathode used; d=interplanar spacing; and θ=diffraction angle). Herein, interplanar spacings, diffraction angle and overall pattern are important for identification of crystal in the X-ray powder diffraction, due to the characteristics of the data. The relative intensity should not be strictly interpreted since it may be varied depending on the direction of crystal growth, particle sizes and measurement conditions. In addition, the diffraction angles usually mean ones which coincide in the range of 2θ±0.2°. The peaks mean main peaks and include peaks not larger than medium at diffraction angles other than those stated above.

The crystalline form of the compound 4-(2,6-dichloro-benzoylamino)-1H-pyrazole-3-carboxylic acid (1-methanesulphonyl-piperidin-4-yl)-amide prepared using the N,N-dimethylacetamide/acetone/water solvent system has been characterised by XRPD and has an X-ray powder diffraction pattern essentially as shown in FIG. 5.

The powder X-ray diffraction patterns are expressed in terms of the diffraction angle (20), inter planar spacing (d) and relative intensities.

Accordingly, in another embodiment, the invention provides, for any of the therapeutic uses of the invention as defined herein, a substantially crystalline form of 4-(2,6-dichloro-benzoylamino)-1H-pyrazole-3-carboxylic acid (1-methanesulphonyl-piperidin-4-yl)-amide which has an X-ray powder diffraction pattern characterised by the presence of major peaks at the diffraction angles (2θ) and interplanar spacings (d) set forth in Table A.

TABLE A

| 2θ/° | d/Å | I |
|---|---|---|
| 16.57 | 5.35 | 59 |
| 16.95 | 5.23 | 62 |
| 20.42 | 4.35 | 76 |
| 22.66 | 3.92 | 100 |
| 24.33 | 3.66 | 40 |

The X-ray powder diffraction pattern is preferably further characterised by the presence of additional peaks at the diffraction angles (2θ) and interplanar spacings (d) set forth in Table B.

TABLE B

| 2θ/° | d/Å | I |
|---|---|---|
| 5.63 | 15.70 | 24 |
| 12.56 | 7.05 | 26 |
| 13.35 | 6.63 | 27 |
| 14.89 | 5.95 | 18 |
| 19.53 | 4.55 | 37 |
| 20.88 | 4.25 | 23 |
| 24.99 | 3.56 | 16 |

The invention further provides, for any of the therapeutic uses of the invention as defined herein, a substantially crystalline form of 4-(2,6-dichloro-benzoylamino)-1H-pyrazole-3-carboxylic acid (1-methanesulphonyl-piperidin-4-yl)-amide which exhibits peaks at the same diffraction angles as those of the X-ray powder diffraction pattern shown in FIG. 5. Preferably the peaks have the same relative intensity as the peaks in FIG. 5.

In a preferred embodiment, the invention provides, for any of the therapeutic uses of the invention as defined herein, a substantially crystalline form of 4-(2,6-dichloro-benzoylamino)-1H-pyrazole-3-carboxylic acid (1-methanesulphonyl-piperidin-4-yl)-amide which has an X-ray powder diffraction pattern substantially as shown in FIG. 5.

The crystalline form of the compound of the invention can also be characterised by differential scanning calorimetry (DSC).

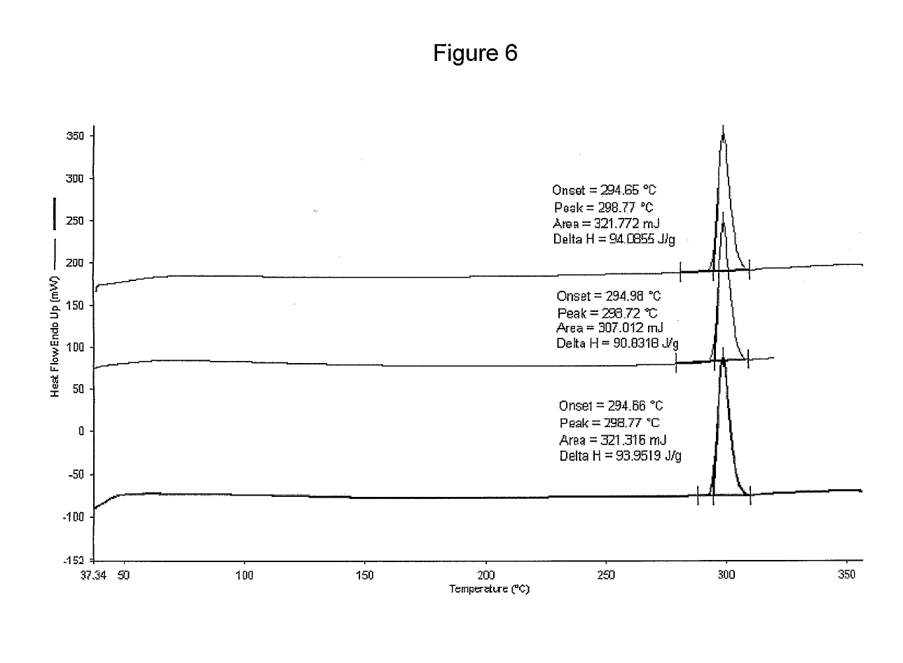

The crystalline form of 4-(2,6-dichloro-benzoylamino)-1H-pyrazole-3-carboxylic acid (1-methanesulphonyl-piperidin-4-yl)-amide prepared using the N,N-dimethylacetamide/acetone/water solvent system has been analysed by DSC and exhibits an endothermic peak at 293-296° C., for example 294.5-295° C., indicative of the thermally induced melting of the crystalline lattice. No significant transitions were apparent prior to the main melting endotherm thus indicating that the crystalline form of the compound of the invention is anhydrous. The DSC scan is shown in FIG. 6.

Accordingly, in another aspect, the invention provides, for any of the therapeutic uses of the invention as defined herein, a substantially crystalline form of 4-(2,6-dichloro-benzoylamino)-1H-pyrazole-3-carboxylic acid (1-methanesulphonyl-piperidin-4-yl)-amide which is anhydrous and exhibits an endothermic peak at 293-296° C., for example 294.5-295° C. when subjected to DSC.

The crystalline form of 4-(2,6-dichloro-benzoylamino)-1H-pyrazole-3-carboxylic acid (1-methanesulphonyl-piperidin-4-yl)-amide can be further characterised by infra-red spectroscopy, e.g. FTIR.

The infra-red spectrum of the crystalline form of the compound prepared using the N,N-dimethylacetamide/acetone/water solvent system includes characteristic peaks, when analysed using the Universal Attenuated Total Reflectance (UATR) method, at 3362, 3019, 2843, 1677, 1577, 1547, 1533, 1326, 1150, 926, 781, 667 cm⁻¹.

Without wishing to be bound by any theory, it is believed that the infra red peaks can be assigned to structural components of the salt as follow:

| Peak: | Due to: |
|---|---|
| 3361.92 cm⁻¹ | N—H |
| 3018.97 cm⁻¹ | aromatic C—H |
| 2842.99 cm⁻¹ | aliphatic C—H |
| 1676.72 cm⁻¹ | amide C═O |
| 1577.31, 1546.92, 1532.94 cm⁻¹ | amide |
| 1325.63 cm⁻¹ | aromatic C—N |
| 1149.91 cm⁻¹ | 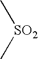 |
| 925.73 cm⁻¹ | C—H aromatic |
| 780.75, 666.88 cm⁻¹ | aromatic C—H |

Accordingly, in a further embodiment, the invention provides, for any of the therapeutic uses of the invention as defined herein, a substantially crystalline form of 4-(2,6-dichloro-benzoylamino)-1H-pyrazole-3-carboxylic acid (1-methanesulphonyl-piperidin-4-yl)-amide which exhibits an infra-red spectrum when analysed using the Universal Attenuated Total Reflectance (UATR) method, containing characteristic peaks at 3362, 3019, 2843, 1677, 1577, 1547, 1533, 1326, 1150, 926, 781, 667 cm⁻¹.

As will be evident from the foregoing paragraphs, the novel crystalline form of the compound of the invention can be characterised by a number of different physicochemical parameters. Accordingly, in a preferred embodiment, the invention provides, for any of the therapeutic uses of the invention as defined herein, a substantially crystalline form of 4-(2,6-dichloro-benzoylamino)-1H-pyrazole-3-carboxylic acid (1-methanesulphonyl-piperidin-4-yl)-amide which is characterised by any one or more (in any combination) or all of the following parameters, namely that the crystalline form:

(a) has a crystal structure as set out in FIGS. 3 and 4; and/or
(b) has a crystal structure as defined by the coordinates in Table 1 herein; and/or
(c) has crystal lattice parameters at a=9.15, b=31.32, c=7.93 Å, β=113.3°, α=γ=90°; and/or
(d) has a crystal structure that belongs belong to a monoclinic space group such as C2/c (# 15); and/or
(e) has an X-ray powder diffraction pattern characterised by the presence of major peaks at the diffraction angles (2θ) and interplanar spacings (d) set forth in Table A, and optionally Table B; and/or
(f) exhibits peaks at the same diffraction angles as those of the X-ray powder diffraction pattern shown in FIG. 5 and optionally wherein the peaks have the same relative intensity as the peaks in FIG. 5; and/or
(g) has an X-ray powder diffraction pattern substantially as shown in FIG. 5; and/or
(h) is anhydrous and exhibits an endothermic peak at an endothermic peak at 293-296° C., for example 294.5-295° C. when subjected to DSC; and/or
(i) exhibits an infra-red spectrum, when analysed using the Universal Attenuated Total Reflectance (UATR) method, that contains characteristic peaks at containing characteristic peaks at 3362, 3019, 2843, 1677, 1577, 1547, 1533, 1326, 1150, 926, 781, 667 cm⁻¹.

### Biological Activity of the Compounds of Formulae (0) and (I′″) and Uses Thereof.

The compounds of the formulae (0), (I′″) and sub-groups thereof are inhibitors of cyclin dependent kinases. For example, compounds for use in the combinations of the invention are inhibitors of cyclin dependent kinases, and in particular CDK5.

As a consequence of their activity in modulating or inhibiting CDK kinases, they will be useful for treating pain or conditions such as stroke.

### Pain

The range of pain sensations experienced and multiple mechanisms involved make a precise definition of pain difficult, therefore in the present invention the term “pain” is used in the broadest sense to describe a spectrum of conditions including nociceptive pain, arising from tissue damage or inflammation, pain related to noxious stimuli, acute pain, chronic pain, and neuropathic pain.

In the present description the terms “treatment” or “treat” refer to both prophylactic or preventative treatment as well as curative or palliative treatment of pain, in particular anti-nociceptive and anti-allodynic treatment of pain.

Examples of types of pain for which the compounds of the present invention will be useful in treating include nociception, somatic pain, visceral pain, acute pain, chronic pain, hyperalgesia, allodynia, post operative pain, pain due to hypersensivity, headache, inflammatory pain (rheumatic, dental, dysmenorrhoea or infection), neurological pain, musculoskeletal pain, cancer related pain or vascular pain.

In one embodiment, the pain may be other than cancer pain.

In another embodiment, the pain may be cancer pain, For example, the cancer pain may be cancer pain resulting from structural damage, periosteal irritation, and nerve entrapment which is the most common complication of both benign and metastatic bone disease, and presents a significant problem in both hospital and community practice (Coleman, 1997, Cancer 80; 1588-1594). In another embodiment the cancer related pain is pain associated with cancer therapy, e.g. postchemotherapy syndromes, chronic postsurgical pain syndromes, post radiation syndromes or bone cancer pain.

One subgroup of types of pain includes nociception, somatic pain, visceral pain, acute pain, chronic pain, hyperalgesia, allodynia, post operative pain, pain due to hypersensivity, headache, inflammatory pain (rheumatic, dental, dysmenorrhoea or infection), neurological pain, musculoskeletal pain or vascular pain.

The pain may be pain associated with a disease or pathological condition in a mammal.

Therefore in one embodiment of the invention is used for the direct treatment of pain in diseases and medical conditions.

Acute pain is that generally short lived with a specific origin e.g. soft tissue damage/trauma (including post surgical pain), inflammation or infection, usually with no persistent psychological reaction. Acute pain can be modulated by analgesics or treatment of the underlying condition e.g. antibiotics to treat infection.

Chronic pain is a more complex condition involving persistent pain over long periods with, sometimes with no apparent cause and with no apparent biological purpose. Chronic pain can often have psychological consequences. Common causes of chronic pain include low-back pain, headache, pain associated with cancer, arthritis pain and fibromyalgia or myofascial pain.

Neuropathic pain is distinct from “normal” or nociceptive pain, usually results from neurological dysfunction and has a complex and variable etiology. It is often characterised by hyperalgesia (lowered pain threshold and enhanced perception) and allodynia (innocuous thermal or mechanical stimuli causing a perception of pain). Neuropathic pain often fails to respond to the same drugs as nociceptive conditions and is therefore more difficult to treat. Neuropathic pain can arise whenever nerves are damaged by trauma or amputation, disease (herpes zoster, diabetes, cancer), or chemical injury (e.g. as a side effect of drug treatment with nucleotide anti-HIV or some antineoplastic drugs). Examples would include monoradiculopathies, trigeminal neuralgia, post herpetic neuralgia, complex regional pain syndromes and peripheral neuropathies.

Peripheral neuropathy is a neurodegenerative condition affecting peripheral nerves usually manifesting as one or a combination of motor, sensory, sensorimotor, or autonomic dysfunction. Peripheral neuropathies can result from disease e.g. diabetes (diabetic neuropathy), alcoholism, acquired immunodeficiency syndrome (AIDS), drug therapies e.g. treatment with cytostatics or genetic predisposition (e.g. Metachromatic leukodystrophy). Peripheral neuropathies are often accompanied by pain conditions.

In addition, the compounds of formula (0) and (I′″) can be used inter alia in the treatment of pain conditions such as acute and chronic pain (as well as, but not limited to, pain associated with cancer, surgery, arthritis, dental surgery, trauma, musculo-skeletal injury or disease, visceral diseases) and migraine headache. Additionally the painful conditions can be neuropathic; examples of such conditions are post-herpetic neuralgia, diabetic neuropathy, drug-induced neuropathy, HIV-mediated neuropathy, sympathetic reflex dystrophy or causalgia, fibromyalgia, myofacial pain, entrapment neuropathy, phantom limb pain and trigeminal neuralgia. Neuropathic conditions include central pain related to stroke, multiple sclerosis, spinal cord injury, arachnoiditis, neoplasms, syringomyelia, Parkinson's disease and epilepsia.

Another sub-group of pain conditions includes all of the pain conditions listed in the preceding paragraph other than cancer pain, i.e. pain associated with cancer.

The present invention is particularly applicable to the palliative treatment of pain, i.e. the direct relief of pain in addition to the relief of pain as the result of amelioration of the underlying disease or medical condition, which is the cause of the pain. Thus, advantageously the invention provides methods and uses for the direct analgesic or acute treatment of pain.

The potential activity of the compounds in treating pain conditions may be tested using a variety of well known techniques. Examples of such techniques include observations of spontaneous pain (ie gait analysis/spontaneous foot lifting/weight bearing), evoked elements (e.g. heat (Hargreaves test and hot plate test), cold (application of acetone), paw pressure test (Randal Siletoe test) or mechanical (von Frey hairs) stimuli or rat tail clip test) or similar/equivalent assays, in test species exposed to the test compound in comparison to appropriate controls.

These models could be further modified to improve sensitivity or to test inflammatory pain behaviour by injection of an inflammatory agent (formalin, carageenan, capsaicin, complete Freud's adjuvant, or incomplete Freud's adjuvant) given intra-plantar or intra-articular prior to testing. Activity of the compounds in neuropathic pain conditions could be evaluated using the “Chung” model of peripheral neuropathy (Kim S H, Chung J M., Pain 1992; 50: 355-363). In vivo electro-physiological single cell recordings or nerve fibre recordings could be employed to measure spontaneous and evoked firing rates. Immunohistochemical evidence e.g. staining for substance P, cGRP, galanin, or other relevant substances might also be used.

The activity of the compounds in treating pain is considered to arise from their activity as inhibitors of cyclin dependent kinase 5 (CDK5). Such activity can be measured using the assay set forth in the examples below and the level of activity exhibited by a given compound can be defined in terms of the IC₅₀ value. Preferred compounds for use in the present invention are compounds having an IC₅₀ value of less than 1 micromolar, more preferably less than 0.1 micromolar.

### Stroke

The compounds of formula (0) and subgroups thereof such as formula (I′″) may also be used to treat patients suffering from stroke or at risk of suffering from stroke.

Where a patient is suffering from stroke, the compounds of the invention may be administered to provide a neuroprotective effect to prevent or reduce the extent of damage to brain issue.

For example, the compounds of the invention may be used to treat ischemic stroke, which is the most common type of stroke, and which results from insufficient cerebral circulation of blood caused by obstruction of the inflow of arterial blood.

The ischemic stroke may be caused by, for example, a thrombus, i.e., a blood clot that forms in a blood vessel. The thrombus may interrupt arterial blood flow, causing brain ischemia and consequent neurologic symptoms. The thrombus may be one which arises as a result of inflammation or atherosclerosis.

Ischemic stroke may also be caused by the lodging of an embolus (an air bubble) from the heart in an intracranial vessel, causing decreased perfusion pressure or increased blood viscosity with inadequate cerebral blood flow. An embolus may be caused by various disorders, including atrial fibrillation and atherosclerosis.

The compounds of the invention may also be used to treat hemorrhagic stroke, which is a form of stroke involving a hemorrhage or rupture of an artery leading to the brain. Hemorrhagic stroke results in bleeding into brain tissue, including the epidural, subdural, or subarachnoid space of the brain. A hemorrhagic stroke typically results from the rupture of an arteriosclerotic vessel that has been exposed to arterial hypertension or to thrombosis.

During acute ischemic stroke, i.e., the period from the cerebrovascular event up to 24 hours after the event, the arterial occlusion results in an immediate infarcted core of brain tissue, where cerebral blood flow is significantly reduced, for example to less than 20% of the normal blood flow. The infarcted core suffers irreversible damage due to significant cell death. The length of time that ischemia persists, and the severity of the ischemia, contribute to the extent of injury. An area around the infracted core, known as the ischemic penumbra, suffers a delayed and less severe infarct. For example, during acute stroke the penumbra may have a reduction in blood flow of from about 20-40% of normal blood flow. The compounds of the invention will be useful in reducing neuronal cell death due to ischemia.

The compounds of the invention may also be used for the prevention or reduction of risk of stroke in patients at risk for stroke. For example, the patients may exhibit any one or more risk factors selected from vascular inflammation, atherosclerosis, arterial hypertension, diabetes, hyperlipidemia and atrial fibrillation.

The compounds of the invention may be administered to facilitate recovery or restoration after an acute stroke period, for example through the reduction or prevention of secondary cell damage in the penumbra.

The potential usefulness of the compounds of formula (0) and sub-groups thereof such as formula (I′″) arises from their ability to modulate (e.g inhibit) cdk kinase activity and in particular Cdk kinase 4, 5 & 6 which have been shown to be involved with or mediate neuronal death following hypoxic or ischemic insult.

In the context of the use of the compounds in treating stroke, preferred compounds of the present invention are compounds having an IC₅₀ value of less than 1 micromolar, more preferably less than 0.1 micromolar.

### Polycystic Kidney Disease

The compounds of formula (0) and subgroups thereof such as formula (I′″) may be used for the treatment of polycystic kidney disease (PKD or for the prevention or treatment of cyst formation elsewhere in the body.

The treatment of PKD may take various forms.

For example, compounds of the invention may be used to prevent or slow down progression of PKD where the existence of PKD in a patient has been confirmed.

The compounds of the invention may be used for the treatment of progressive renal insufficiency associated with the progression of cystic disease.

Alternatively, or additionally, compounds of the invention may be administered so as to prevent or slow down the development of one or more symptoms of PKD. Examples of such symptoms include hypertension associated with PKD, bleeding into the cyst, or pain associated with cyst expansion.

In one particular form, the invention provides a method for the treatment of hypertension accompanying polycystic kidney disease (PKD) by administering to a patient an effective amount of a compound of the invention.

Although the compounds of the invention may be used to treat cystic or polycystic conditions elsewhere in the body, the disease to be treated is preferably polycystic kidney disease (PKD).

The PKD can be autosomal dominant polycystic kidney disease (ADPKD) or autosomal recessive polycystic kidney disease (ARPKD) as described in the introductory sections of this application.

As indicated in the introductory sections of this application, ADPKD is caused by mutations in one of three genes: namely chromosome PKD1 on chromosome 16, PKD2 on chromosome 4 and the as yet unmapped PKD3 gene. Patients susceptible to the disease can be diagnosed by identifying mutations in the PKD1, PKD2 or PKD3 genes. The patients can be diagnosed prior to the development of clinically significant symptoms of PKD or they can be diagnosed once clinically significant symptoms have been detected.

The diagnoses can be carried out using standard methods well known to the skilled person. Standard methods of identification and analysis of mutations include direct sequencing, oligonucleotide microarray analysis, a mutant specific antibody or by using reverse-transcriptase polymerase chain reaction (RT-PCR) or in-situ hybridization such as fluorescence in situ hybridization (FISH) techniques. The diagnostic tests are typically conducted on a biological sample selected from blood samples (isolation and enrichment of shed tumour cells), stool biopsies, sputum, chromosome analysis, pleural fluid, peritoneal fluid, buccal spears, biopsy or urine. Once diagnosed, the patients can be treated with a compound of the invention.

In screening by RT-PCR, the level of mRNA in the tumour is assessed by creating a cDNA copy of the mRNA followed by amplification of the cDNA by PCR. Methods of PCR amplification, the selection of primers, and conditions for amplification, are known to a person skilled in the art. Nucleic acid manipulations and PCR are carried out by standard methods, as described for example in Ausubel, F. M. et al., eds. Current Protocols in Molecular Biology, 2004, John Wiley & Sons Inc., or Innis, M. A. et-al., eds. PCR Protocols: a guide to methods and applications, 1990, Academic Press, San Diego. Reactions and manipulations involving nucleic acid techniques are also described in Sambrook et al., 2001, 3rd Ed, Molecular Cloning: A Laboratory Manual, Cold Spring Harbor Laboratory Press. Alternatively a commercially available kit for RT-PCR (for example Roche Molecular Biochemicals) may be used, or methodology as set forth in U.S. Pat. Nos. 4,666,828; 4,683,202; 4,801,531; 5,192,659, 5,272,057, 5,882,864, and 6,218,529 and incorporated herein by reference.

An example of an in-situ hybridisation technique for assessing mRNA expression would be fluorescence in-situ hybridisation (FISH) (see Angerer, 1987 Meth. Enzymol., 152: 649). Generally, in situ hybridization comprises the following major steps: (1) fixation of tissue to be analyzed; (2) prehybridization treatment of the sample to increase accessibility of target nucleic acid, and to reduce nonspecific binding; (3) hybridization of the mixture of nucleic acids to the nucleic acid in the biological structure or tissue; (4) post-hybridization washes to remove nucleic acid fragments not bound in the hybridization, and (5) detection of the hybridized nucleic acid fragments. The probes used in such applications are typically labeled, for example, with radioisotopes or fluorescent reporters. Preferred probes are sufficiently long, for example, from about 50, 100, or 200 nucleotides to about 1000 or more nucleotides, to enable specific hybridization with the target nucleic acid(s) under stringent conditions. Standard methods for carrying out FISH are described in Ausubel, F. M. et al., eds. Current Protocols in Molecular Biology, 2004, John Wiley & Sons Inc and Fluorescence In Situ Hybridization: Technical Overview by John M. S. Bartlett in Molecular Diagnosis of Cancer, Methods and Protocols, 2nd ed.; ISBN: 1-59259-760-2; March 2004, pps. 077-088; Series: Methods in Molecular Medicine.

More specifically, the patient may be subjected to a diagnostic test to detect mutated forms PDK1, PDK2 or PDK3, or to detect a marker characteristic of a mutated form PDK1, PDK2 or PDK3. By marker we include genetic markers including, for example, the measurement of DNA composition to identify mutations. The term marker also includes markers which are characteristic of up regulation including enzyme activity, enzyme levels, enzyme state and mRNA levels of the aforementioned proteins. The term up-regulation includes elevated expression or over-expression, including gene amplification (i.e. multiple gene copies) and increased expression by a transcriptional effect, and hyperactivity and activation, including activation by mutations. From a clinical perspective patients may have palpable flank masses, elevated blood pressure and abnormally large quantities of protein in the urine. Diagnosis can therefore be based upon imaging using for example MRI or CT or ultrasound or pyelograms, and/or genetic testing as described herein.

The compounds of the invention can be used in a curative or palliative sense to treat the PKD and/or its symptoms once the symptoms of the disease have become apparent.

Alternatively, the compounds of the invention can be used in a prophylactic sense to treat patients who have been tested and determined as suffering from a mutation in the PK\D1 and/or PKD2 and/or PKD3 gene as described above, but who have not yet developed clinically significant symptoms.

### Methods for the Preparation of Compounds of the Formula (0)

Compounds of the Formula (0) can be prepared as described in WO 2005/012256 at pages 91-101.

In this section, as in all other sections of this application unless the context indicates otherwise, references to formula (0) also include all sub-groups and examples thereof as defined herein such as formula (I′″). Where a reference is made to a group R¹ and R³ or any other “R” group, the definition of the group in question is as set out above and as set out in the following sections of this application unless the context requires otherwise.

Compounds of the formula (0) can be prepared in accordance with synthetic methods well known to the skilled person, and by methods set out below and as described in our application PCT/GB2004/003179 (WO 2005/012256), the contents of which are incorporated herein by reference.

For example, compounds of the formula (0) can be prepared by the sequence of reactions shown in Scheme 1.

The starting material for the synthetic route shown in Scheme 1 is the 4-nitro-pyrazole-3-carboxylic acid (X) which can either be obtained commercially or can be prepared by nitration of the corresponding 4-unsubstituted pyrazole carboxy compound.

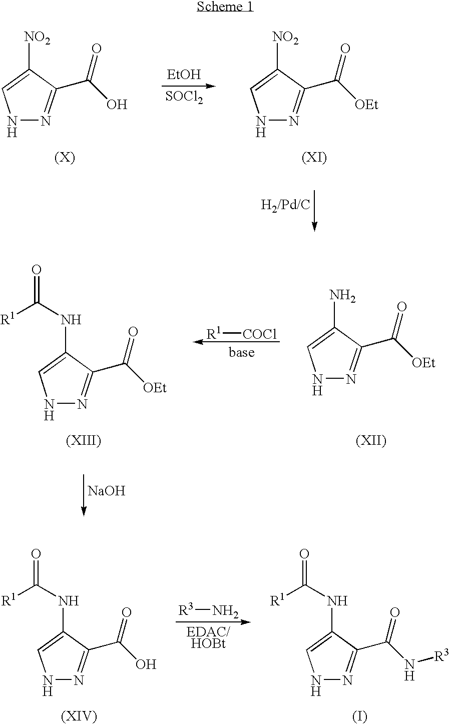

The nitro-pyrazole carboxylic acid (X) is converted to the corresponding ester (XI), for example the methyl or ethyl ester (of which the ethyl ester is shown), by reaction with the appropriate alcohol such as ethanol in the presence of an acid catalyst or thionyl chloride. The reaction may be carried out at ambient temperature using the esterifying alcohol as the solvent.

The nitro-ester (XI) can be reduced to the corresponding amine (XII) by standard methods for converting a nitro group to an amino group. Thus, for example, the nitro group can be reduced to the amine by hydrogenation over a palladium on charcoal catalyst. The hydrogenation reaction can be carried out in a solvent such as ethanol at ambient temperature.

The resulting amine (XII) can be converted to the amide (XIII) by reaction with an acid chloride of the formula R¹COCl in the presence of a non-interfering base such as triethylamine. The reaction may be carried out at around room temperature in a polar solvent such as dioxan. The acid chloride can be prepared by treatment of the carboxylic acid R¹CO₂H with thionyl chloride, or by reaction with oxalyl chloride in the presence of a catalytic amount of dimethyl formamide, or by reaction of a potassium salt of the acid with oxalyl chloride.

As an alternative to using the acid chloride method described above, the amine (XII) can be converted to the amide (XIII) by reaction with the carboxylic acid R¹CO₂H in the presence of amide coupling reagents of the type commonly used in the formation of peptide linkages. Examples of such reagents include 1,3-dicyclohexylcarbodiimide (DCC) (Sheehan et al, J. Amer. Chem. Soc. 1955, 77, 1067), 1-ethyl-3-(3′-dimethylaminopropyl)-carbodiimide (referred to herein either as EDC or EDAC but also known in the art as EDCI and WSCDI) (Sheehan et al, J. Org. Chem., 1961, 26, 2525), uronium-based coupling agents such as O-(7-azabenzotriazol-1-yl)-N,N,N′,N′-tetramethyluronium hexafluorophosphate (HATU) and phosphonium-based coupling agents such as 1-benzo-triazolyloxytris-(pyrrolidino)phosphonium hexafluorophosphate (PyBOP) (Castro et al, Tetrahedron Letters, 1990, 31, 205). Carbodiimide-based coupling agents are advantageously used in combination with 1-hydroxy-7-azabenzotriazole (HOAt) (L. A. Carpino, J. Amer. Chem. Soc., 1993, 115, 4397) or 1-hydroxybenzotriazole (HOBt) (Konig et al, Chem. Ber., 103, 708, 2024-2034). Preferred coupling reagents include EDC (EDAC) and DCC in combination with HOAt or HOBt.

The coupling reaction is typically carried out in a non-aqueous, non-protic solvent such as acetonitrile, dioxan, dimethylsulphoxide, dichloromethane, dimethylformamide or N-methylpyrrolidine, or in an aqueous solvent optionally together with one or more miscible co-solvents. The reaction can be carried out at room temperature or, where the reactants are less reactive (for example in the case of electron-poor anilines bearing electron withdrawing groups such as sulphonamide groups) at an appropriately elevated temperature. The reaction may be carried out in the presence of a non-interfering base, for example a tertiary amine such as triethylamine or N,N-diisopropylethylamine.

The amide (XIII) is subsequently hydrolysed to the carboxylic acid (XIV) by treatment with an aqueous alkali metal hydroxide such sodium hydroxide. The saponification reaction may be carried out using an organic co-solvent such as an alcohol (e.g. methanol) and the reaction mixture is typically heated to a non-extreme temperature, for example up to about 50-60° C.

The carboxylic acid (XIV) can then be converted to a compound of the formula (I′″) by reaction with an amine R³—NH₂ using the amide forming conditions described above. Thus, for example, the amide coupling reaction may be carried out in the presence of EDC and HOBt in a polar solvent such as DMF.

An alternative general route to compounds of the formula (I′″) wherein R^{2b} is hydrogen is shown in Scheme 2.

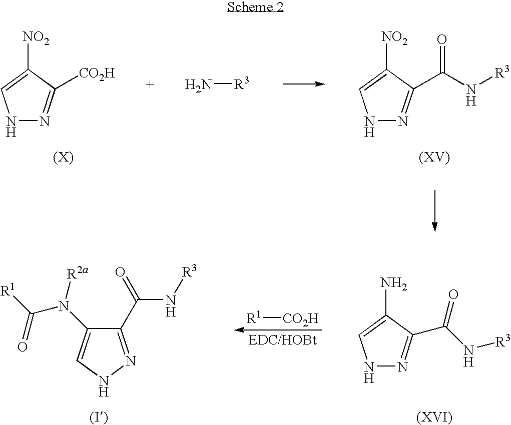

In Scheme 2, the nitro-pyrazole-carboxylic acid (X), or an activated derivative thereof such as an acid chloride, is reacted with amine R³—NH₂ using the amide forming conditions described above to give the nitro-pyrazole-amide (XV) which is then reduced to the corresponding amino compound (XVI) using a standard method of reducing nitro groups, for example the method involving hydrogenation over a Pd/C catalyst as described above.

The amine (XVI) is then coupled with a carboxylic acid of the formula R¹—CO₂H or an activated derivative thereof such as an acid chloride or anhydride under the amide-forming conditions described above in relation to Scheme 1. Thus, for example, as an alternative to using an acid chloride, the coupling reaction can be carried out in the presence of EDAC (EDC) and HOBt in a solvent such as DMF to give a compound of the formula (I′) which corresponds to a compound of the formula (I′″) wherein R^{2b} is hydrogen.

Compounds of the formula (I′″) can also be prepared from a compound of the formula (XVII):

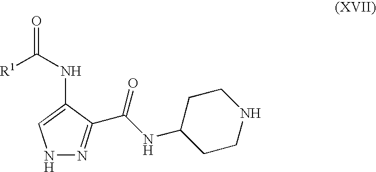

by reaction with an appropriate sulphonylating agent, for example a sulphonyl chloride such as methanesulphonyl chloride.

An illustrative reaction sequence showing the conversion of a compound of the formula (XVII) into sulphonyl derivatives of the formula (I′″) is set out in Scheme 3.

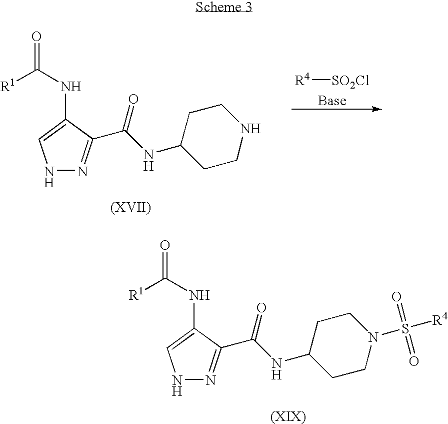

As shown in Scheme 3, a compound of the formula (I′″) in which R³ is a piperidine ring bearing a sulphonyl group —SO₂R⁴ (i.e. a compound of the formula (XIX)) can be prepared by reacting the compound of the formula (XVII) with a sulphonyl chloride R⁴SO₂Cl (such as methane sulphonyl chloride) in the presence of a non-interfering base such as diisopropylethylamine. The reaction is typically carried out at room temperature in a non-aqueous non-protic solvent such as dioxane and dichloromethane.

The sulphonyl chlorides of the formula R⁴SO₂Cl may be obtained from commercial sources, or can be prepared by a number of procedures. For example, alkylsulphonyl chlorides can be prepared by reacting an alkyl halide with sodium sulphite with heating in an aqueous organic solvent such as water/dioxane to form the corresponding sulphonic acid followed by treatment with thionyl chloride in the presence of DMF to give the sulphonyl chloride.

In an alternative preparation, a thiol R⁴SH/R^{4a}SH can be reacted with potassium nitrate and sulphuryl chloride to give the required sulphonyl chloride.

In many of the reactions described above, it may be necessary to protect one or more groups to prevent reaction from taking place at an undesirable location on the molecule. Examples of protecting groups, and methods of protecting and deprotecting functional groups, can be found in Protective Groups in Organic Synthesis (T. Green and P. Wuts; 3rd Edition; John Wiley and Sons, 1999).

For example, an amine group may be protected as an amide (—NRCO—R) or a urethane (—NRCO—OR), for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), or as a 2(-phenylsulphonyl)ethyloxy amide (—NH-Psec). Other protecting groups for amines, such as cyclic amines and heterocyclic N—H groups, include toluenesulphonyl (tosyl) and methanesulphonyl (mesyl) groups and benzyl groups such as a para-methoxybenzyl (PMB) group. A carboxylic acid group may be protected as an ester for example, as: a C₁₋₇ alkyl ester (e.g., a methyl ester; a t-butyl ester); a C₁₋₇ haloalkyl ester (e.g., a C₁₋₇ trihaloalkyl ester); a tri-C₁₋₁₇ alkylsilyl-C₁₋₇alkyl ester; or a C₅₋₂₀ aryl-C₁₋₁₇ alkyl ester (e.g., a benzyl ester; a nitrobenzyl ester); or as an amide, for example, as a methyl amide. A thiol group may be protected, for example, as a thioether (—SR), for example, as: a benzyl thioether; an acetamidomethyl ether (—S—CH₂NHC(═O)CH₃).

### Methods of Purification of the Compounds

The compounds may be isolated and purified by a number of methods well known to those skilled in the art and examples of such methods include chromatographic techniques such as column chromatography (e.g. flash chromatography) and HPLC. Preparative LC-MS is a standard and effective method used for the purification of small organic molecules such as the compounds described herein. The methods for the liquid chromatography (LC) and mass spectrometry (MS) can be varied to provide better separation of the crude materials and improved detection of the samples by MS. Optimisation of the preparative gradient LC method will involve varying columns, volatile eluents and modifiers, and gradients. Methods are well known in the art for optimising preparative LC-MS methods and then using them to purify compounds. Such methods are described in Rosentreter U. Huber U.; Optimal fraction collecting in preparative LC/MS; J Comb Chem.; 2004; 6(2), 159-64 and Leister W. Strauss K, Wisnoski D, Zhao Z. Lindsley C., Development of a custom high-throughput preparative liquid chromatography/mass spectrometer platform for the preparative purification and analytical analysis of compound libraries; J Comb Chem.; 2003; 5(3); 322-9.

One such system for purifying compounds via preparative LC-MS is described in the experimental section below although a person skilled in the art will appreciate that alternative systems and methods to those described could be used. In particular, normal phase preparative LC based methods might be used in place of the reverse phase methods described here. Most preparative LC-MS systems utilise reverse phase LC and volatile acidic modifiers, since the approach is very effective for the purification of small molecules and because the eluents are compatible with positive ion electrospray mass spectrometry. Employing other chromatographic solutions e.g. normal phase LC, alternatively buffered mobile phase, basic modifiers etc as outlined in the analytical methods described above could alternatively be used to purify the compounds.

### Pharmaceutical Formulations

While it is possible for the active compounds in the combinations of the invention to be administered alone, it is preferable to present them as a pharmaceutical composition (e.g. formulation) comprising at least one active compound together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents; for example agents that reduce or alleviate some of the side effects associated with chemotherapy

Thus, the present invention further provides pharmaceutical compositions, as defined above, and methods of making a pharmaceutical composition comprising admixing at least one active compound, as defined above, together with a compound and one or more pharmaceutically acceptable carriers, excipients, buffers, adjuvants, stabilizers, or other materials, as described herein.

The term “pharmaceutically acceptable” as used herein pertains to combinations, compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Accordingly, in a further aspect, the invention provides combinations comprising (or consisting essentially of) a compounds of the formula (0) and sub-groups thereof as defined herein in the form of pharmaceutical compositions.

The pharmaceutical compositions can be in any form suitable for oral, parenteral, topical, intranasal, ophthalmic, otic, rectal, intra-vaginal, or transdermal administration. Where the compositions are intended for parenteral administration, they can be formulated for intravenous, intramuscular, intraperitoneal, subcutaneous administration or for direct delivery into a target organ or tissue by injection, infusion or other means of delivery. The delivery can be by bolus injection, short term infusion or longer term infusion and can be via passive delivery or through the utilisation of a suitable infusion pump.

Pharmaceutical formulations adapted for parenteral administration include aqueous and non-aqueous sterile injection solutions which may contain anti-oxidants, buffers, bacteriostats, co-solvents, organic solvent mixtures, cyclodextrin complexation agents, emulsifying agents (for forming and stabilizing emulsion formulations), liposome components for forming liposomes, gellable polymers for forming polymeric gels, lyophilisation protectants and combinations of agents for, inter alia, stabilising the active ingredient in a soluble form and rendering the formulation isotonic with the blood of the intended recipient. Pharmaceutical formulations for parenteral administration may also take the form of aqueous and non-aqueous sterile suspensions which may include suspending agents and thickening agents (R. G. Strickly, Solubilizing Excipients in oral and injectable formulations, Pharmaceutical Research, Vol 21(2) 2004, p 201-230).

A drug molecule that is ionizable can be solubilized to the desired concentration by pH adjustment if the drug's pKₐ is sufficiently away from the formulation pH value. The acceptable range is pH 2-12 for intravenous and intramuscular administration, but subcutaneously the range is pH 2.7-9.0. The solution pH is controlled by either the salt form of the drug, strong acids/bases such as hydrochloric acid or sodium hydroxide, or by solutions of buffers which include but are not limited to buffering solutions formed from glycine, citrate, acetate, maleate, succinate, histidine, phosphate, tris(hydroxymethyl)aminomethane (TRIS), or carbonate.

The combination of an aqueous solution and a water-soluble organic solvent/surfactant (i.e., a cosolvent) is often used in injectable formulations. The water-soluble organic solvents and surfactants used in injectable formulations include but are not limited to propylene glycol, ethanol, polyethylene glycol 300, polyethylene glycol 400, glycerin, dimethylacetamide (DMA), N-methyl-2-pyrrolidone (NMP; Pharmasolve), dimethylsulphoxide (DMSO), Solutol HS 15, Cremophor EL, Cremophor RH 60, and polysorbate 80. Such formulations can usually be, but are not always, diluted prior to injection.

Propylene glycol, PEG 300, ethanol, Cremophor EL, Cremophor RH 60, and polysorbate 80 are the entirely organic water-miscible solvents and surfactants used in commercially available injectable formulations and can be used in combinations with each other. The resulting organic formulations are usually diluted at least 2-fold prior to IV bolus or IV infusion.

Alternatively increased water solubility can be achieved through molecular complexation with cyclodextrins

The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use.

Liposomes are closed spherical vesicles composed of outer lipid bilayer membranes and an inner aqueous core and with an overall diameter of <100 μm. Depending on the level of hydrophobicity, moderately hydrophobic drugs can be solubilized by liposomes if the drug becomes encapsulated or intercalated within the liposome. Hydrophobic drugs can also be solubilized by liposomes if the drug molecule becomes an integral part of the lipid bilayer membrane, and in this case, the hydrophobic drug is dissolved in the lipid portion of the lipid bilayer. A typical liposome formulation contains water with phospholipid at −5-20 mg/ml, an isotonicifier, a pH 5-8 buffer, and optionally cholesterol.

The pharmaceutical formulation can be prepared by lyophilising a compound of formula (I) or acid addition salt thereof. Lyophilisation refers to the procedure of freeze-drying a composition. Freeze-drying and lyophilisation are therefore used herein as synonyms. A typical process is to solubilise the compound and the resulting formulation is clarified, sterile filtered and aseptically transferred to containers appropriate for lyophilisation (e.g. vials). In the case of vials, they are partially stoppered with lyo-stoppers. The formulation can be cooled to freezing and subjected to lyophilisation under standard conditions and then hermetically capped forming a stable, dry lyophile formulation. The composition will typically have a low residual water content, e.g. less than 5% e.g. less than 1% by weight based on weight of the lyophile.

The lyophilisation formulation may contain other excipients for example, thickening agents, dispersing agents, buffers, antioxidants, preservatives, and tonicity adjusters. Typical buffers include phosphate, acetate, citrate and glycine. Examples of antioxidants include ascorbic acid, sodium bisulphite, sodium metabisulphite, monothioglycerol, thiourea, butylated hydroxytoluene, butylated hydroxyl anisole, and ethylenediamietetraacetic acid salts. Preservatives may include benzoic acid and its salts, sorbic acid and its salts, alkyl esters of para-hydroxybenzoic acid, phenol, chlorobutanol, benzyl alcohol, thimerosal, benzalkonium chloride and cetylpyridinium chloride. The buffers mentioned previously, as well as dextrose and sodium chloride, can be used for tonicity adjustment if necessary.

Bulking agents are generally used in lyophilisation technology for facilitating the process and/or providing bulk and/or mechanical integrity to the lyophilized cake. Bulking agent means a freely water soluble, solid particulate diluent that when co-lyophilised with the compound or salt thereof, provides a physically stable lyophilized cake, a more optimal freeze-drying process and rapid and complete reconstitution. The bulking agent may also be utilised to make the solution isotonic.

The water-soluble bulking agent can be any of the pharmaceutically acceptable inert solid materials typically used for lyophilisation. Such bulking agents include, for example, sugars such as glucose, maltose, sucrose, and lactose; polyalcohols such as sorbitol or mannitol; amino acids such as glycine; polymers such as polyvinylpyrrolidine; and polysaccharides such as dextran.

The ratio of the weight of the bulking agent to the weight of active compound is typically within the range from about 1 to about 5, for example of about 1 to about 3, e.g. in the range of about 1 to 2.

Alternatively they can be provided in a solution form which may be concentrated and sealed in a suitable vial. Sterilisation of dosage forms may be via filtration or by autoclaving of the vials and their contents at appropriate stages of the formulation process. The supplied formulation may require further dilution or preparation before delivery for example dilution into suitable sterile infusion packs.

Extemporaneous injection solutions and suspensions may be prepared from sterile powders, granules and tablets.

In one preferred embodiment of the invention, the pharmaceutical composition is in a form suitable for i.v. administration, for example by injection or infusion.

Pharmaceutical compositions of the present invention for parenteral injection can also comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

The compositions of the present invention may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption such as aluminum monostearate and gelatin.

If a compound is not stable in aqueous media or has low solubility in aqueous media, it can be formulated as a concentrate in organic solvents. The concentrate can then be diluted to a lower concentration in an aqueous system, and can be sufficiently stable for the short period of time during dosing. Therefore in another aspect, there is provided a pharmaceutical composition comprising a non aqueous solution composed entirely of one or more organic solvents, which can be dosed as is or more commonly diluted with a suitable IV excipient (saline, dextrose; buffered or not buffered) before administration (Solubilizing excipients in oral and injectable formulations, Pharmaceutical Research, 21(2), 2004, p 201-230). Examples of solvents and surfactants are propylene glycol, PEG300, PEG400, ethanol, dimethylacetamide (DMA), N-methyl-2-pyrrolidone (NMP, Pharmasolve), Glycerin, Cremophor EL, Cremophor RH 60 and polysorbate. Particular non aqueous solutions are composed of 70-80% propylene glycol, and 20-30% ethanol. One particular non aqueous solution is composed of 70% propylene glycol, and 30% ethanol. Another is 80% propylene glycol and 20% ethanol. Normally these solvents are used in combination and usually diluted at least 2-fold before IV bolus or IV infusion. The typical amounts for bolus IV formulations are ˜50% for Glycerin, propylene glycol, PEG300, PEG400, and ˜20% for ethanol. The typical amounts for IV infusion formulations are ˜15% for Glycerin, 3% for DMA, and ˜10% for propylene glycol, PEG300, PEG400 and ethanol.

In one preferred embodiment of the invention, the pharmaceutical composition is in a form suitable for i.v. administration, for example by injection or infusion. For intravenous administration, the solution can be dosed as is, or can be injected into an infusion bag (containing a pharmaceutically acceptable excipient, such as 0.9% saline or 5% dextrose), before administration.

In another preferred embodiment, the pharmaceutical composition is in a form suitable for sub-cutaneous (s.c.) administration.

Pharmaceutical dosage forms suitable for oral administration include tablets, capsules, caplets, pills, lozenges, syrups, solutions, powders, granules, elixirs and suspensions, sublingual tablets, wafers or patches and buccal patches.

Pharmaceutical compositions containing compounds of the formula (I) can be formulated in accordance with known techniques, see for example, Remington's Pharmaceutical Sciences, Mack Publishing Company, Easton, Pa., USA.

Thus, tablet compositions can contain a unit dosage of active compound together with an inert diluent or carrier such as a sugar or sugar alcohol, eg; lactose, sucrose, sorbitol or mannitol; and/or a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (eg; tablets, capsules etc.) can be coated or un-coated, but typically have a coating, for example a protective film coating (e.g. a wax or varnish) or a release controlling coating. The coating (e.g. a Eudragit™ type polymer) can be designed to release the active component at a desired location within the gastrointestinal tract. Thus, the coating can be selected so as to degrade under certain pH conditions within the gastrointestinal tract, thereby selectively release the compound in the stomach or in the ileum or duodenum.

Instead of, or in addition to, a coating, the drug can be presented in a solid matrix comprising a release controlling agent, for example a release delaying agent which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract. As a further alternative, the active compound can be formulated in a delivery system that provides osmotic control of the release of the compound. Osmotic release and other delayed release or sustained release formulations may be prepared in accordance with methods well known to those skilled in the art.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, dragées, tablets or capsules.

Pharmaceutical compositions for oral administration can be obtained by combining the active ingredient with solid carriers, if desired granulating a resulting mixture, and processing the mixture, if desired or necessary, after the addition of appropriate excipients, into tablets, dragee cores or capsules. It is also possible for them to be incorporated into plastics carriers that allow the active ingredients to diffuse or be released in measured amounts.

The compounds for use in the combinations of the invention can also be formulated as solid dispersions. Solid dispersions are homogeneous extremely fine disperse phases of two or more solids. Solid solutions (molecularly disperse systems), one type of solid dispersion, are well known for use in pharmaceutical technology (see (Chiou and Riegelman, J. Pharm. Sci., 60, 1281-1300 (1971)) and are useful in increasing dissolution rates and increasing the bioavailability of poorly water-soluble drugs.

Solid dispersions of drugs are generally produced by melt or solvent evaporation methods. For melt processing, the materials (excipients) which are usually semisolid and waxy in nature, are heated to cause melting and dissolution of the drug substance, followed by hardening by cooling to very low temperatures. The solid dispersion can then be pulverized, sieved, mixed with excipients, and encapsulated into hard gelatin capsules or compressed into tablets. Alternatively the use of surface-active and self-emulsifying carriers allows the encapsulation of solid dispersions directly into hard gelatin capsules as melts. Solid plugs are formed inside the capsules when the melts are cooled to room temperature.

Solid solutions can also be manufactured by dissolving the drug and the required excipient in either an aqueous solution or a pharmaceutically acceptable organic solvent, followed by removal of the solvent, using a pharmaceutically acceptable method, such as spray drying. The resulting solid can be particle sized if required, optionally mixed with exipients and either made into tablets or filled into capsules.

A particularly suitable polymeric auxiliary for producing such solid dispersions or solid solutions is polyvinylpyrrolidone (PVP).

The present invention provides a pharmaceutical composition comprising a substantially amorphous solid solution, said solid solution comprising

(a) a compound of the formula (I), for example the compound of Example 1; and
(b) a polymer selected from the group consisting of:
polyvinylpyrrolidone (povidone), crosslinked polyvinylpyrrolidone (crospovidone), hydroxypropyl methylcellulose, hydroxypropylcellulose, polyethylene oxide, gelatin, crosslinked polyacrylic acid (carbomer), carboxymethylcellulose, crosslinked carboxymethylcellulose (croscarmellose), methylcellulose, methacrylic acid copolymer, methacrylate copolymer, and water soluble salts such as sodium and ammonium salts of methacrylic acid and methacrylate copolymers, cellulose acetate phthalate, hydroxypropylmethylcellulose phthalate and propylene glycol alginate;
wherein the ratio of said compound to said polymer is about 1:1 to about 1:6, for example a 1:3 ratio, spray dried from a mixture of one of chloroform or dichloromethane and one of methanol or ethanol, preferably dichloromethane/ethanol in a 1:1 ratio.

This invention also provides solid dosage forms comprising the solid solution described above. Solid dosage forms include tablets, capsules and chewable tablets. Known excipients can be blended with the solid solution to provide the desired dosage form. For example, a capsule can contain the solid solution blended with (a) a disintegrant and a lubricant, or (b) a disintegrant, a lubricant and a surfactant. A tablet can contain the solid solution blended with at least one disintegrant, a lubricant, a surfactant, and a glidant. The chewable tablet can contain the solid solution blended with a bulking agent, a lubricant, and if desired an additional sweetening agent (such as an artificial sweetener), and suitable flavours.

The pharmaceutical formulations may be presented to a patient in “patient packs” containing an entire course of treatment in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in patient prescriptions. The inclusion of a package insert has been shown to improve patient compliance with the physician's instructions.

Compositions for topical use include ointments, creams, sprays, patches, gels, liquid drops and inserts (for example intraocular inserts). Such compositions can be formulated in accordance with known methods.

Compositions for parenteral administration are typically presented as sterile aqueous or oily solutions or fine suspensions, or may be provided in finely divided sterile powder form for making up extemporaneously with sterile water for injection.

Examples of formulations for rectal or intra-vaginal administration include pessaries and suppositories which may be, for example, formed from a shaped moldable or waxy material containing the active compound.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose.

The compounds of the formula (0)) will generally be presented in unit dosage form and, as such, will typically contain sufficient compound to provide a desired level of biological activity. For example, a formulation may contain from 1 nanogram to 2 grams of active ingredient, e.g. from 1 nanogram to 2 milligrams of active ingredient. Within this range, particular sub-ranges of compound are 0.1 milligrams to 2 grams of active ingredient (more usually from 10 milligrams to 1 gram, e.g. 50 milligrams to 500 milligrams), or 1 microgram to 20 milligrams (for example 1 microgram to 10 milligrams, e.g. 0.1 milligrams to 2 milligrams of active ingredient).

For oral compositions, a unit dosage form may contain from 1 milligram to 2 grams, more typically 10 milligrams to 1 gram, for example 50 milligrams to 1 gram, e.g. 100 miligrams to 1 gram, of active compound.

The compound will be administered to a patient in need thereof (for example a human or animal patient) in an amount sufficient to achieve the desired therapeutic effect.

In one particular embodiment, the composition used in the therapeutic methods of the invention comprises the compound 4-(2,6-dichloro-benzoylamino)-1H-pyrazole-3-carboxylic acid (1-methanesulphonyl-piperidin-4-yl)-amide, where 4-(2,6-dichloro-benzoylamino)-1H-pyrazole-3-carboxylic acid (1-methanesulphonyl-piperidin-4-yl)-amide is a solid pharmaceutical composition comprising a compressed mixture of:

(a) a solid dispersion of 4-(2,6-dichloro-benzoylamino)-1H-pyrazole-3-carboxylic acid (1-methanesulphonyl-piperidin-4-yl)-amide in polyvinylpyrrolidone;
(b) a solid diluent; and
(c) a disintegrant; and optionally
(d) one or more further pharmaceutically acceptable excipients.

The solid pharmaceutical composition is typically presented in tablet or capsule form.

The solid pharmaceutical composition can be in the form of a tablet.

Alternatively the solid pharmaceutical composition is in the form of a capsule.

The solid dispersion (a) contains 4-(2,6-dichloro-benzoylamino)-1H-pyrazole-3-carboxylic acid (1-methanesulphonyl-piperidin-4-yl)-amide dispersed in polyvinylpyrrolidone (PVP). The dispersion may take the form of a solid solution, or may consist of the compound of the invention dispersed as a finely divided solid in a surrounding matrix of PVP.

PVP is available in a range of molecular weights and a particular grade of PVP for use in the formulations of the present invention has a molecular weight in the range from 44,000-54,000.

The solid dispersion typically contains 4-(2,6-dichloro-benzoylamino)-1H-pyrazole-3-carboxylic acid (1-methanesulphonyl-piperidin-4-yl)-amide and the PVP in a weight ratio of about 1:1 to about 1:6, more typically 1:2 to 1:4, for example a 1:3 ratio.

The solid dispersion can be prepared by dissolving 4-(2,6-dichloro-benzoylamino)-1H-pyrazole-3-carboxylic acid (1-methanesulphonyl-piperidin-4-yl)-amide and the PVP in a common solvent (for example a solvent selected from chloroform, dichloromethane, methanol and ethanol and mixtures thereof (e.g. dichloromethane/ethanol in a 1:1 ratio) and then removing the solvent for example on a rotary evaporator or by spray drying, in particular by spray drying the resulting solution.

The spray dried solid dispersion on its own typically has a very low density and the solid diluent assists in increasing the density of the composition, rendering it easier to compress. The solid diluent is typically a pharmacologically inert solid substance chosen from sugars or sugar alcohols, e.g. lactose, sucrose, sorbitol or mannitol; and non-sugar derived diluents such as sodium carbonate, calcium phosphate, calcium carbonate, and cellulose or derivatives thereof such as methyl cellulose, ethyl cellulose, hydroxypropyl methyl cellulose, and starches such as corn starch.

Particular diluents are lactose and calcium phosphate.

The disintegrant is a substance that swells rapidly on contact with water so as to cause the rapid disintegration of the pharmaceutical composition and release of 4-(2,6-dichloro-benzoylamino)-1H-pyrazole-3-carboxylic acid (1-methanesulphonyl-piperidin-4-yl)-amide.

Particular disintegrants are those known in the art as “super disintegrants” and include cross linked carboxymethylcellulose (Croscarmellose), cross-linked polyvinylpyrrolidone (cross-linked PVP or Crospovidone), and sodium starch glycolate. Examples of preferred super disintegrants are Croscarmellose and sodium starch glycolate.

Examples of other pharmaceutically acceptable excipients (d) that may be included in the pharmaceutical compositions of the invention include microcrystalline cellulose, which can act as both a diluent and an auxiliary disintegrant. Silicified microcrystalline cellulose (which contains about 1-3% silicon dioxide, typically about 2% silicon dioxide), may also be used to enhance the flowability of the composition and thereby improve the ease with which the composition can be compressed.

Another pharmaceutically acceptable excipient (d) that can be included in the compressed mixture is an alkali metal bicarbonate such as sodium bicarbonate. The bicarbonate reacts with acid in the stomach to release carbon dioxide thereby facilitating more rapid disintegration of the pharmaceutical composition.

One particular mixture of components (a) to (d) is a mixture wherein:
component (a) is a spray dried solid dispersion of 4-(2,6-dichloro-benzoylamino)-1H-pyrazole-3-carboxylic acid (1-methanesulphonyl-piperidin-4-yl)-amide in PVP in a ratio of 1:3;
component (b) is calcium phosphate;
component (c) is Croscarmellose; and
component (d) is silicified microcrystalline cellulose.

The mixture of components (a) to (c) and optionally (d) is compressed prior to processing to give the final dosage form. Thus, for example, it can be compressed to give a compressed solid mass (e.g. in the form of a ribbon or pellet) and then milled to form granules of a desired particle size. The granules can then be filled into a capsule or shaped and compressed to form a tablet.

The mixture of components (a) to (c) and optionally (d) can be compressed by means of various methods well known to the skilled person. For example, they can be compressed using a roller compactor to form a ribbon which can then be broken up and milled to form granules.

In one embodiment, the composition comprises 4-(2,6-dichloro-benzoylamino)-1H-pyrazole-3-carboxylic acid (1-methanesulphonyl-piperidin-4-yl)-amide, where 4-(2,6-dichloro-benzoylamino)-1H-pyrazole-3-carboxylic acid (1-methanesulphonyl-piperidin-4-yl)-amide is in a pharmaceutical composition in the form of a capsule containing a milled compressed mixture of components (a) to (c) and optionally (d) as defined herein.

In another embodiment, the composition comprises 4-(2,6-dichloro-benzoylamino)-1H-pyrazole-3-carboxylic acid (1-methanesulphonyl-piperidin-4-yl)-amide, where 4-(2,6-dichloro-benzoylamino)-1H-pyrazole-3-carboxylic acid (1-methanesulphonyl-piperidin-4-yl)-amide is in a pharmaceutical composition in the form of a tablet comprising a compressed mixture of components (a) to (c) and optionally (d) as defined herein.

### Methods of Treatment

The compounds of formula (0) and sub-formulae thereof such as formula (I′″) may be used to treat pain conditions in patients. Prior to treatment, a diagnosis of the pain condition will be carried out by someone skilled in the art. This could include obtaining history and characteristics of the pain, physical examination of the patient and any appropriate diagnostic tests. Once the type of pain has been determined, a compound of formula (0) may be administered in an amount effective to treat the pain.

As stated above, the terms “treatment” and “treat” in the context of pain include both prophylactic and palliative treatment. Thus, the compounds of formula (0) may be used in a prophylactic sense to prevent the onset of pain or to prevent pain from worsening, or they may be used to reduce or eliminate pain in a patient suffering from pain.

The compounds may also be used to treat or reduce the effects of stroke as described above. For example, the compounds may be used as neuroprotective agents to prevent or reduce the damage to brain tissue following a stroke. They may also be administered to patients exhibiting one or more risk factors indicative of a possible stroke.

Furthermore, the compounds may be used for the prevention or treatment of cystic diseases and in particular cystic renal diseases such as polycystic kidney disease.

Thus, the compounds of formula (0) and subgroups thereof such as formula (I′″) are typically administered to a subject in need of such administration, for example a human or animal patient, preferably a human.

The compounds are typically administered in amounts that are therapeutically or prophylactically useful and which generally are non-toxic. However, in certain situations (for example in the case of extreme pain or pain associated with a terminal condition), the benefits of administering the compounds may outweigh the disadvantages of any toxic effects or side effects, in which case it may be considered desirable to administer the compound in amounts that are associated with a degree of toxicity.

The compounds of the invention may be administered over a prolonged term to maintain beneficial therapeutic effects or may be administered for a short period only. Alternatively they may be administered in a pulsatile or continuous manner.

A typical daily dose of the compound of formula (0) can be in the range from 100 picograms to 100 milligrams per kilogram of body weight, more typically 5 nanograms to 25 milligrams per kilogram of bodyweight, and more usually 10 nanograms to 15 milligrams per kilogram (e.g. 10 nanograms to 10 milligrams, and more typically 1 microgram per kilogram to 20 milligrams per kilogram, for example 1 microgram to 10 milligrams per kilogram) per kilogram of bodyweight although higher or lower doses may be administered where required. The compound of the formula (I) can be administered on a daily basis for example.

The compounds of the invention may be administered orally in a range of doses, for example 1 to 1500 mg, 2 to 800 mg, or 5 to 500 mg, e.g. 2 to 200 mg or 10 to 1000 mg, particular examples of doses including 10, 20, 50 and 80 mg. The compounds may be administered once or more than once each day depending on the severity and type of pain.

Ultimately, however, the quantity of compound administered and the type of composition used will be commensurate with the nature of the disease or physiological condition being treated and will be at the discretion of the physician.

Accordingly, a person skilled in the art would know through their common general knowledge the dosing regimes to use.

The compounds of formula (0) and sub-formulae thereof such as formula (I′″) can be administered as a sole therapeutic agent or in combination with other therapeutic agents. For example, the compounds can be administered together with one or more other therapeutic agents useful for treating pain. Examples include other anti-nociceptive compounds, non-steroidal anti-inflammatories (NSAID's), opioids, GABA analogues, narcotic analgesics, local anaesthetics, NMDA antagonists, neuroleptic agents, anti-convulsants, anti-spasmodics, anti depressants or muscle relaxants and/or excipients/formulations to treat the pain conditions described.

### EXAMPLES

The invention will now be illustrated, but not limited, by reference to the specific embodiments described in the following examples. The starting materials for each of the Examples are commercially available unless otherwise specified.

In the examples, the compounds prepared were characterised by liquid chromatography and mass spectroscopy (LC-MS) using the system and operating conditions set out below. Where chlorine is present and a single mass is quoted, the mass quoted for the compound is for ³⁵Cl. The two systems were equipped with identical chromatography columns and were set up to run under the same operating conditions. The operating conditions used are also described below. In the examples, the retention times are given in minutes.

In the examples, the following abbreviations may be used.
AcOH acetic acid
BOC tert-butyloxycarbonyl
CDI 1,1-carbonyldiimidazole
DMAW90 Solvent mixture: DCM: MeOH, AcOH, H₂O (90:18:3:2)
DMAW120 Solvent mixture: DCM: MeOH, AcOH, H₂O (120:18:3:2)
DMAW240 Solvent mixture: DCM: MeOH, AcOH, H₂O (240:20:3:2)
DCM dichloromethane
DMF dimethylformamide
DMSO dimethyl sulphoxide
EDC 1-ethyl-3-(3′-dimethylaminopropyl)-carbodiimide
Et₃N triethylamine
EtOAc ethyl acetate
Et₂O diethyl ether
HOAt 1-hydroxyazabenzotriazole
HOBt 1-hydroxybenzotriazole
MeCN acetonitrile
MeOH methanol
P.E. petroleum ether
SiO₂ silica
TBTU N,N,N′,N′-tetramethyl-O-(benzotriazol-1-yl)uronium tetrafluoroborate
THF tetrahydrofuran

### Platform System

| | |
|---|---|
| System: | Waters 2790/Platform LC |
| Mass Spec Detector: | Micromass Platform LC |
| PDA Detector: | Waters 996 PDA |

### Analytical Conditions:

| | |
|---|---|
| Eluent A: | 5% CH3CN in 95% H₂O (0.1% Formic Acid) |
| Eluent B: | CH₃CN (0.1% Formic Acid) |
| Gradient: | 10-95% eluent B |
| Flow: | 1.2 ml/min |
| Column: | Synergi 4 μm Max-RP C₁₂, 80A, 50 × 4.6 mm (Phenomenex) |

### MS Conditions:

| | | |
|---|---|---|
| Capillary voltage: | 3.5 | kV |
| Cone voltage: | 30 | V |
| Source Temperature: | 120° | C. |

### FractionLynx System

| | |
|---|---|
| System: | Waters FractionLynx (dual analytical/prep) |
| Mass Spec Detector: | Waters-Micromass ZQ |
| PDA Detector: | Waters 2996 PDA |

### Analytical Conditions:

| | |
|---|---|
| Eluent A: | H₂O (0.1% Formic Acid) |
| Eluent B: | CH₃CN (0.1% Formic Acid) |
| Gradient: | 5-95% eluent B |
| Flow: | 1.5 ml/min |
| Column: | Synergi 4 μm Max-RP C₁₂, 80A, 50 × 4.6 mm (Phenomenex) |

### MS Conditions:

| | | |
|---|---|---|
| Capillary voltage: | 3.5 | kV |
| Cone voltage: | 30 | V |
| Source Temperature: | 120° | C. |
| Desolvation Temperature: | 300° | C. |

### Analytical LC-MS System

Several systems were used, as described below, and these were equipped with were set up to run under closely similar operating conditions. The operating conditions used are also described below.

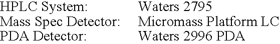

### Acidic Analytical Conditions

| | |
|---|---|
| Eluent A: | H₂O (0.1% Formic Acid) |
| Eluent B: | CH₃CN (0.1% Formic Acid) |
| Gradient: | 5-95% eluent B over 3.5 minutes |
| Flow: | 0.8 ml/min |
| Column: | Phenomenex Synergi 4μ MAX-RP 80A, 2.0 × 50 mm |

### Basic Analytical Conditions:

| | |
|---|---|
| Eluent A: | H₂O (10 mM NH₄HCO₃ buffer adjusted to pH = 9.5 with |
| | NH₄OH) |
| Eluent B: | CH₃CN |
| Gradient: | 05-95% eluent B over 3.5 minutes |
| Flow: | 0.8 ml/min |
| Column: | Thermo Hypersil-Keystone BetaBasic-18 5 μm 2.1 × 50 mm |
| or | |
| Column: | Phenomenex Luna C18(2) 5 μm 2.0 × 50 mm |

### Polar Analytical Conditions:

| | |
|---|---|
| Eluent A: | H₂O (0.1% Formic Acid) |
| Eluent B: | CH₃CN (0.1% Formic Acid) |
| Gradient: | 00-50% eluent B over 3 minutes |
| Flow: | 0.8 ml/min |
| Column: | Thermo Hypersil-Keystone HyPurity Aquastar, 5μ, |
| | 2.1 × 50 mm |
| or | |
| Column: | Phenomenex Synergi 4μ MAX-RP 80A, 2.0 × 50 mm or |

### Longer Analytical Conditions:

| | |
|---|---|
| Eluent A: | H₂O (0.1% Formic Acid) |
| Eluent B: | CH₃CN (0.1% Formic Acid) |
| Gradient: | 05-95% eluent B over 15 minutes |
| Flow: | 0.4 ml/min |
| Column: | Phenomenex Synergi 4μ MAX-RP 80A, 2.0 × 150 mm |

### MS Conditions:

| | |
|---|---|
| Capillary voltage: | 3.6 kV |
| Cone voltage: | 30 V |
| Source Temperature: | 120° C. |
| Scan Range: | 165-700 amu |
| Ionisation Mode: | ElectroSpray Positive or |
| | ElectroSpray Negative or |
| | ElectroSpray Positive & Negative |

### Mass Directed Purification LC-MS System

The following preparative chromatography systems can be used to purify the compounds of the invention.

Hardware:

Waters Fractionlynx system:

### 2767 Dual Autosampler/Fraction Collector

2525 preparative pump
CFO (column fluidic organiser) for column selection
RMA (Waters reagent manager) as make up pump

### Waters ZQ Mass Spectrometer

Waters 2996 Photo Diode Array detector

| | |
|---|---|
| Software: | Masslynx 4.0 |

Columns:

1. Low pH chromatography: Phenomenex Synergy MAX-RP, 10μ, 150×15 mm (alternatively used same column type with 100×21.2 mm dimensions).
2. High pH chromatography: Phenomenex Luna C18 (2), 10μ, 100×21.2 mm (alternatively used Thermo Hypersil Keystone BetaBasic C18, 5μ, 100×21.2 mm)

Eluents:

### 1. Low pH Chromatography:

### Solvent A: H₂O+0.1% Formic Acid, pH 1.5

### Solvent B: CH₃CN+0.1% Formic Acid

### 2. High pH Chromatography:

### Solvent A: H₂O+10 mM NH₄HCO₃+NH₄OH, pH 9.5

### Solvent B: CH₃CN

3. Make up solvent: MeOH+0.1% formic acid (for both chromatography type)

Methods:

Prior to using preparative chromatography to isolate and purify the product compounds, analytical LC-MS (see above) can first be used to determine the most appropriate conditions for preparative chromatography. A typical routine is to run an analytical LC-MS using the type of chromatography (low or high pH) most suited for compound structure. Once the analytical trace shows good chromatography, a suitable preparative method of the same type can be chosen. Typical running condition for both low and high pH chromatography methods are:

Flow rate: 24 ml/min
Gradient: Generally all gradients have an initial 0.4 min step with 95% A+5% B. Then according to analytical trace a 3.6 min gradient is chosen in order to achieve good separation (e.g. from 5% to 50% B for early retaining compounds; from 35% to 80% B for middle retaining compounds and so on)
Wash: 1 minute wash step is performed at the end of the gradient
Re-equilibration: A 2.1 minute re-equilibration step is carried out to prepare the system for the next run
Make Up flow rate: 1 ml/min

Solvent:

All compounds were usually dissolved in 100% MeOH or 100% DMSO

MS Running Conditions:

| | |
|---|---|
| Capillary voltage: | 3.2 kV |
| Cone voltage: | 25 V |
| Source Temperature: | 120° C. |
| Multiplier: | 500 V |
| Scan Range: | 125-800 amu |
| Ionisation Mode: | ElectroSpray Positive |

The starting materials for each of the Examples are commercially available unless otherwise specified.

### Example 1

### General Procedure A

### Preparation of Amide from Amino-Pyrazole

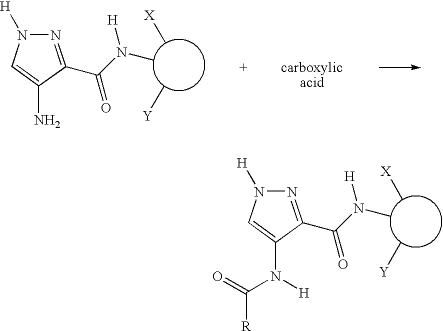

To a stirred solution of the appropriate 4-amino-1H-pyrazole-3-carboxylic acid amide (0.23 mmol), EDAC (52 mg; 0.27 mmol) and HOBt (37 mg; 0.27 mmol) in 5 ml of N,N-dimethylformamide was added the corresponding carboxylic acid (0.25 mmol), and the mixture was then left at room temperature overnight. The reaction mixture was evaporated and the residue purified by preparative LC/MS, to give the product.

### General Procedure B

### Deprotection of Piperidine Ring Nitrogen by Removal of tert-Butoxycarbonyl Group

A product of Procedure A containing a piperidine group bearing an N-tert-butoxycarbonyl (t-Boc) protecting group (40 mg) was treated with saturated ethyl acetate/HCl, and stirred at room temperature for 1 hour. A solid precipitated out of the reaction mixture, which was filtered off, washed with ether, and then dried to give 25 mg product (LC/MS: [M+H]⁺ 364).

| | | | |
|---|---|---|---|
| Example 1 | 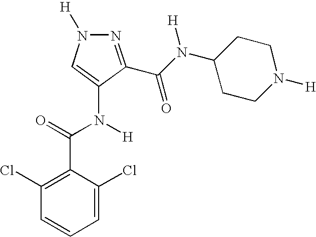 | Procedure A followed by Procedure B | [M + H]⁺ 383 Rₜ 1.87 |

### Example 2

### Preparation of 4-(2,6-dichloro-benzoylamino)-1H-pyrazole-3-carboxylic acid piperidin-4-ylamide hydrochloride

### 2A. 4-(2,6-dichloro-benzoylamino)-1H-pyrazole-3-carboxylic acid

2,6-dichlorobenzoyl chloride (8.2 g; 39.05 mmol) was added cautiously to a solution of 4-amino-1H-pyrazole-3-carboxylic acid methyl ester (prepared in a manner analogous to 165B) (5 g; 35.5 mmol) and triethylamine (5.95 ml; 42.6 mmol) in dioxan (50 ml) then stirred at room temperature for 5 hours. The reaction mixture was filtered and the filtrate treated with methanol (50 ml) and 2M sodium hydroxide solution (100 ml), heated at 50° C. for 4 hours, and then evaporated. 100 ml of water was added to the residue then acidified with concentrated hydrochloric acid. The solid was collected by filtration, washed with water (100 ml) and sucked dry to give 10.05 g of 4-(2,6-dichloro-benzoylamino)-1H-pyrazole-3-carboxylic acid as a pale violet solid.

### 2B. 4-{[4-(2,6-dichloro-benzoylamino)-1H-pyrazole-3-carbonyl]-amino}-piperidine-1-carboxylic acid tert-butyl ester

A mixture of 4-(2,6-dichloro-benzoylamino)-1H-pyrazole-3-carboxylic acid (6.5 g, 21.6 mmol), 4-amino-1-BOC-piperidine (4.76 g, 23.8 mmol), EDC (5.0 g, 25.9 mmol) and HOBt (3.5 g, 25.9 mmol) in DMF (75 ml) was stirred at room temperature for 20 hours. The reaction mixture was reduced in vacuo and the residue partitioned between ethyl acetate (100 ml) and saturated aqueous sodium bicarbonate solution (100 ml). The organic layer was washed with brine, dried (MgSO₄) and reduced in vacuo. The residue was taken up in 5% MeOH-DCM (˜30 ml). The insoluble material was collected by filtration and, washed with DCM and dried in vacuo to give 4-{[4-(2,6-dichloro-benzoylamino)-1H-pyrazole-3-carbonyl]-amino}-piperidine-1-carboxylic acid tert-butyl ester (5.38 g) as a white solid. The filtrate was reduced in vacuo and the residue purified by column chromatography using gradient elution 1:2 EtOAc/hexane to EtOAc to give further 4-{[4-(2,6-dichloro-benzoylamino)-1H-pyrazole-3-carbonyl]-amino}-piperidine-1-carboxylic acid tert-butyl ester (2.54 g) as a white solid.

### 2C. 4-(2,6-dichloro-benzoylamino)-1H-pyrazole-3-carboxylic acid piperidin-4-ylamide

A solution of 4-{[4-(2,6-dichloro-benzoylamino)-1H-pyrazole-3-carbonyl]-amino}-piperidine-1-carboxylic acid tert-butyl ester (7.9 g) in MeOH (50 mL) and EtOAc (50 ml) was treated with sat. HCl-EtOAc (40 mL) then stirred at r.t. overnight. The product did not crystallise due to the presence of methanol, and therefore the reaction mixture was evaporated and the residue triturated with EtOAc. The resulting off white solid was collected by filtration, washed with EtOAc and sucked dry on the sinter to give 6.3 g of 4-(2,6-dichloro-benzoylamino)-1H-pyrazole-3-carboxylic acid piperidin-4-ylamide as the hydrochloride salt. (LC/MS: Rₜ 5.89, [M+H]⁺ 382/384).

### Example 3

### Preparation of 4-(2,6-dichloro-benzoylamino)-1H-pyrazole-3-carboxylic acid piperidin-4-ylamide acetic acid salt

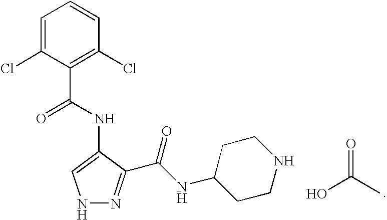

To a solution of 4-(2,6-dichloro-benzoylamino)-1H-pyrazole-3-carboxylic acid piperidin-4-ylamide hydrochloride salt (Example 2C) 20.6 g, 50 mmol) in water (500 ml) stirring at ambient temperature was added sodium bicarbonate (4.5 g, 53.5 mmol). The mixture was stirred for 1 hour and the solid formed collected by filtration and dried in vacuo azeotroping with toluene (×3) to give the corresponding free base of 4-(2,6-dichloro-benzoylamino)-1H-pyrazole-3-carboxylic acid piperidin-4-ylamide.

¹H NMR (400 MHz, DMSO-d₆) δ 10.20 (s, 1H), 8.30 (s, 1H), 8.25 (d, 1H), 7.60-7.50 (m, 3H), 3.70 (m, 1H), 3.00 (d, 2H), 2.50 (m, 2H), 1.70 (d, 2H), 1.50 (m, 2H).

To a stirred suspension of 4-(2,6-dichloro-benzoylamino)-1H-pyrazole-3-carboxylic acid piperidin-4-ylamide (10.0 g, 26.2 mmol) in methanol (150 ml) was added glacial acetic acid (15 ml, 262 mmol) at ambient temperature. After 1 h, a clear solution was obtained which was reduced in vacuo azeotroping with toluene (×2). The residue was then triturated with acetonitrile (2×100 ml) and the solid dried in vacuo to give 4-(2,6-dichloro-benzoylamino)-1H-pyrazole-3-carboxylic acid piperidin-4-ylamide acetic acid salt (10.3 g) as a white solid.

¹H NMR (400 MHz, DMSO-d₆) δ 10.20 (s, 1H), 8.40 (d, 1H), 8.35 (s, 1H), 7.60-7.50 (m, 3H), 3.85 (m, 1H), 3.00 (d, 2H), 2.60 (t, 2H), 1.85 (s, 3H), 1.70 (d, 2H), 1.55 (m, 2H)

### Example 4

### Synthesis of the methanesulphonic acid salt of 4-(2,6-dichloro-benzoylamino)-1H-pyrazole-3-carboxylic acid piperidin-4-ylamide

The methanesulphonic acid salt of 4-(2,6-dichloro-benzoylamino)-1H-pyrazole-3-carboxylic acid piperidin-4-ylamide may be prepared by the synthetic route shown in the Scheme below.

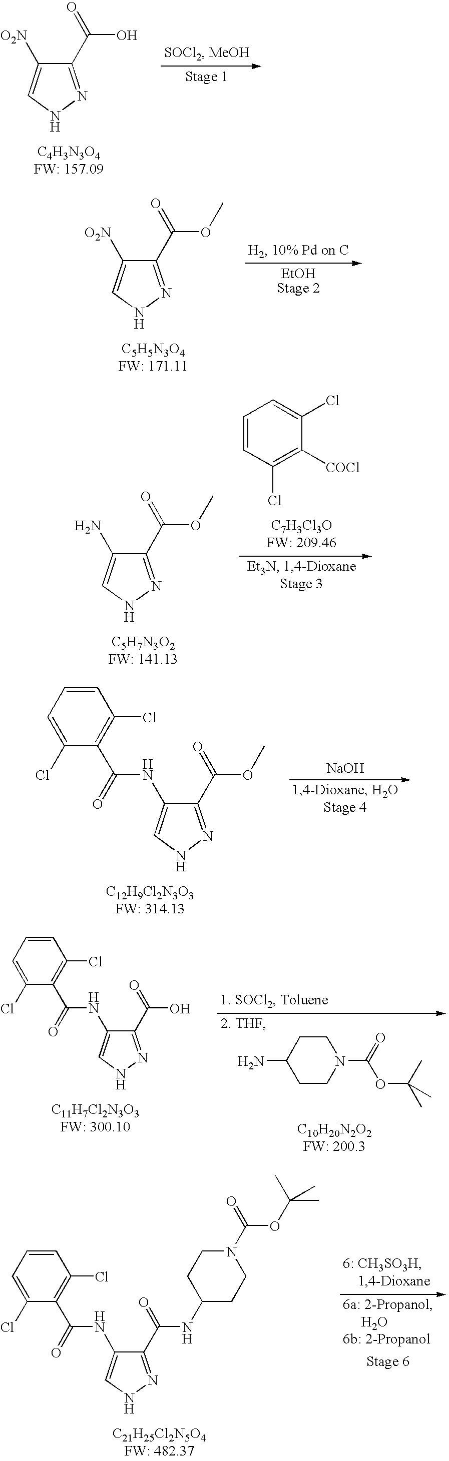

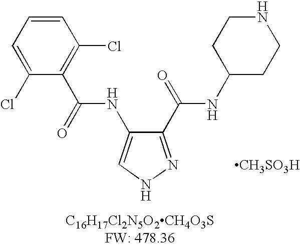

### Stage 1: Preparation of 4-nitro-1H-pyrazole-3-carboxylic acid methyl ester

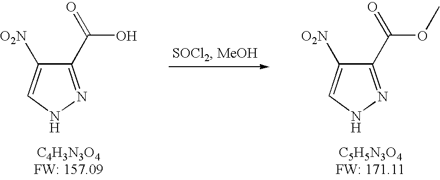

A 20 L reaction vessel equipped with a digital thermometer and stirrer was charged with 4-nitro-1H-pyrazole-3-carboxylic acid (1.117 Kg, 7.11 mol, 1 wt) and methanol (8.950 L, 8 vol). The reaction mixture was stirred under nitrogen, cooled to 0 to 5° C., thionyl chloride (0.581 L, 8.0 mol, 0.52 vol) added over 180 minutes and the resultant mixture allowed to warm to and stir at 18 to 22° C. overnight, after which time ¹H NMR analysis (d₆-DMSO) indicated reaction completion. The reaction mixture was concentrated under reduced pressure at 40 to 45° C., the residue treated with toluene and re-concentrated (3×2.250 L, 3×2 vol) under reduced pressure at 40 to 45° C. to give 4-nitro-1H-pyrazole-3-carboxylic acid methyl ester as an off-white solid (1.210 Kg, 99.5%).

### Stage 2: Preparation of 4-amino-1H-pyrazole-3-carboxylic acid methyl ester

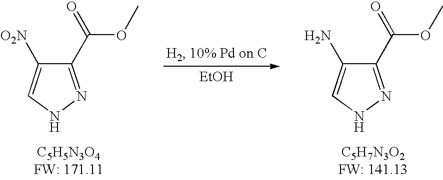

A 20 L reaction vessel equipped with a digital thermometer and stirrer was charged with palladium on carbon (10% wet paste, 0.170 Kg, 0.14 wt) under nitrogen. In a separate vessel a slurry of 4-nitro-1H-pyrazole-3-carboxylic acid methyl ester (1.210 Kg, 7.07 mol, 1 wt) in ethanol (12.10 L, 10 vol) was warmed to 30 to 35° C. to effect dissolution and the solution added to the catalyst under nitrogen. Following a nitrogen-hydrogen purge sequence an atmosphere of hydrogen was introduced and the reaction mixture maintained at 28 to 30° C. until reaction completion (5 to 10 hours) was noted by ¹H NMR analysis (d₆-DMSO). Following a purge cycle, the reaction mixture under nitrogen was filtered and the liquors concentrated under reduced pressure to give 4-amino-1H-pyrazole-3-carboxylic acid methyl ester (0.987 Kg, 98.9%).

### Stage 3: Preparation of 4-(2,6-dichlorobenzoylamino)-1H-pyrazole-3-carboxylic acid methyl ester

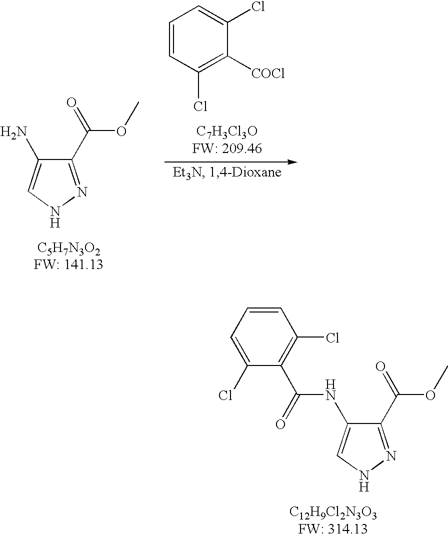

A solution of 4-amino-1H-pyrazole-3-carboxylic acid methyl ester (0.634 Kg, 4.49 mol, 1 wt) in 1,4-dioxane (8.90 L, 9 vol) under nitrogen was treated with triethylamine (0.761 L, 5.46 mol, 1.2 vol) followed by 2,6-dichlorobenzoyl chloride (0.710 L, 4.96 mol, 0.72 vol) such that the internal temperature was maintained in the range 20 to 25° C. Residual 2,6-dichlorobenzoyl chloride was washed in with a line rinse of 1,4-dioxane (0.990 L, 1 vol) and the reaction mixture stirred at 18 to 25° C. until complete (16 hours) by TLC analysis (eluent: ethyl acetate: heptanes 3:1; R_{f amine} 0.25, R_{f product} 0.65). The reaction mixture was filtered, the filter-cake washed with 1,4-dioxane (2×0.990 L, 2×1 vol) and the combined filtrates (red) progressed to Stage 4 without further isolation.

### Stage 4: Preparation of 4-(2,6-dichlorobenzoylamino)-1H-pyrazole-3-carboxylic acid

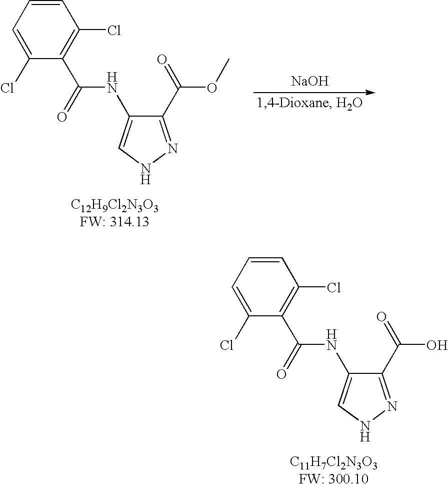

To a solution of sodium hydroxide (0.484 Kg, 12.1 mol) in water (6.05 L) was charged a solution of the Stage 3 ester in one portion: (1.099 Kg, 3.50 mol in 6.00 L). The reaction mixture was stirred to completion at 20 to 25° C. as determined by TLC analysis (eluent: ethyl acetate:heptanes 3:1; R_{f ester} 0.65, R_{f Stage 4} baseline). The reaction mixture was concentrated under reduced pressure at 45 to 50° C., the oily residue diluted with water (9.90 L) and acidified to pH 1 with concentrated hydrochloric acid such that the temperature was maintained below 30° C. The resulting precipitate was collected by filtration, washed with water (5.00 L), pulled dry on the filter and subsequently washed with heptanes (5.00 L). The filter-cake was charged to a 20 L rotary evaporator flask and drying completed azeotropically with toluene (2×4.50 L) to afford 4-(2,6-dichlorobenzoylamino)-1H-pyrazole-3-carboxylic acid as a yellow solid (1.044 Kg, approx. 99.5%).

### Stage 5: Preparation of 4-{[4-(2,6-dichlorobenzoylamino)-1H-pyrazole-3-carbonyl]amino}piperidine-1-carboxylic acid tert-butyl ester

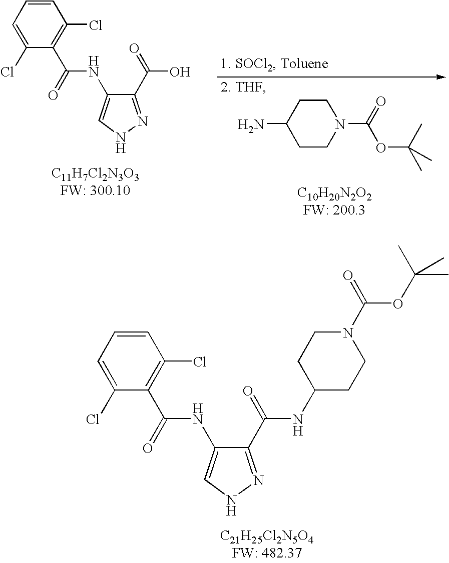

Stage 4 product (1.0 wt) and toluene (10.0 vol) were charged to a suitably sized flange flask equipped with a mechanical stirrer, dropping funnel and thermometer. The contents were stirred under nitrogen at 16 to 25° C. and thionyl chloride (0.3 vol) was added slowly. The contents were then heated to 80 to 100° C. and stirred at this temperature until the reaction was judged complete by ¹H NMR. Further toluene (up to 10 vol) could be added at this stage if the contents were to become too thick to stir. Once complete, the mixture was cooled to between 40 and 50° C. and then concentrated under vacuum at 45 to 50° C. to dryness. The residue was then azeo-dried with toluene (3×2.0 vol).

The isolated solid was transferred to a suitably sized flask and tetrahydrofuran (5.0 vol) was charged. The contents were stirred under nitrogen at 16 to 25° C. and triethylamine (0.512 vol) was added. To a separate flask was charged 4-amino-piperidine-1-carboxylic acid tert-butyl ester (0.704 wt) and tetrahydrofuran (5.0 vol). The contents were agitated until complete dissolution was achieved and the solution was then charged to the reaction flask, maintaining the temperature between 16 and 30° C. The reaction mixture was then heated to between 45 and 50° C. and the contents stirred until judged complete by ¹H NMR. The contents were then cooled to between 16 and 25° C. and water (5.0 vol) was charged.

Mixed heptanes (0.5 vol) were added, the contents were stirred for up to 10 minutes and the layers were separated. The aqueous phase was then extracted with tetrahydrofuran:mixed heptanes [(9:1), 3×5.0 vol]. The organic phases were combined, washed with water (2.5 vol) and then concentrated under vacuum at 40 to 45° C. The residue was azeotroped with toluene (3×5.0 vol) and concentrated to dryness to yield the crude Stage 5 product.

The solid was then transferred to a suitably sized flask, methanol: toluene [(2.5:97.5), 5.0 vol] was added and the slurry was stirred under nitrogen for 3 to 18 hours. The contents were filtered, the filter-cake was washed with toluene (2×0.7 vol) and the solid was then dried under vacuum at 40 to 50° C. to yield 4-{[4-(2,6-dichlorobenzoylamino)-1H-pyrazole-3-carbonyl]amino}piperidine-1-carboxylic acid tert-butyl ester as an off-white solid.

Two batches of Stage 4 product (0.831 kg per batch) were processed in this way to give a total of 2.366 kg (88.6% yield) of 4-{[4-(2,6-dichlorobenzoylamino)-1H-pyrazole-3-carbonyl]amino}piperidine-1-carboxylic acid tert-butyl ester.

### Stage 6: Preparation of 4-(2,6-dichlorobenzoylamino)-1H-pyrazole-3-carboxylic acid piperidin-4-ylamide methanesulphonate

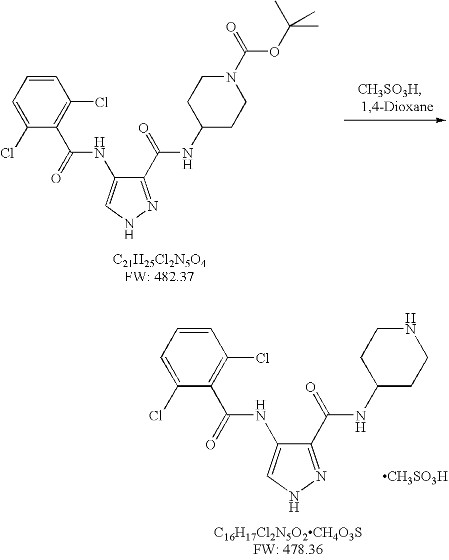

Stage 5 product (1.0 wt) and 1,4-dioxane (30.0 vol) were charged to a suitably sized flange flask equipped with a mechanical stirrer, dropping funnel and thermometer. The contents were stirred under nitrogen and heated to between 80 and 90° C. Methanesulphonic acid (0.54 vol) was added over 30 to 60 minutes and the contents were then heated to 95 to 105° C. and stirred in this temperature range until the reaction was judged complete by ¹H NMR. Once complete, the contents were cooled to between 20 and 30° C. and the resultant precipitate collected by filtration. The filter-cake was washed with 2-propanol (2×2.0 vol) and pulled dry on the filter for 3 to 24 hours to give crude 4-(2,6-dichlorobenzoylamino)-1H-pyrazole-3-carboxylic acid piperidin-4-ylamide methanesulphonate as a free-flowing off-white solid (80.0 to 120.0% w/w, uncorrected for impurities or solutes).

Several batches of Stage 5 product were processed in this way and the details of the quantities of starting material and product for each batch are set out in Table 1 below.

TABLE 1
Yields from the deprotection step - Stage 6

| | Input (g) of (4-{[4-(2,6- | | |
|---|---|---|---|
| | Dichloro-benzoylamino)- | Output (g) of [4-(2,6- | |
| | 1H-pyrazole-3- | Dichlorobenzoyl-amino)- | Chemical |
| | carbonyl]amino}- | 1H-pyrazole-3-carboxylic | purity |
| | piperidine-1-carboxylic | acid piperidin-4-ylamide | (HPLC % |
| Batch | acid tert-butyl ester) | methanesulphonate] | area) |
| 1 | 590.0 | 579.6 | 97.88 |
| | | 99.1% th, 98.2% w/w | |
| 2 | 521.0 | 532.7 | 98.09 |
| | | 103.1% th, 102.2% w/w | |
| 3 | 523.8 | 511.7 | 98.17 |
| | | 98.5% th, 97.7% w/w | |
| 4 | 518.4 | 596.3 | 98.24 |
| | | 116.0% th, 115.0% w/w | |
| 5 | 563.2 | 600.1 | 98.16 |
| | | 107.4% th, 106.6% w/w | |
| 6 | 563.1 | 565.2 | 98.49 |
| | | 101.2% th, 100.4% w/w | |
| 7 | 560.4 | 553.9 | 98.70 |
| | | 99.7% th, 98.8% w/w | |
| 8 | 569.7 | 560.6 | 98.41 |
| | | 99.2% th, 98.4% w/w | |

### Stage 6a: Recrystallisation of 4-(2,6-dichlorobenzoylamino)-1H-pyrazole-3-carboxylic acid piperidin-4-ylamide methanesulphonate

The product of Stage 6 was recrystallised to ensure that any residual levels of Boc-protected product of Stage 5 were no greater than 0.25%. Four batches of Stage 6 product were recrystallised using the following protocol.

Crude Stage 6 product and 2-propanol (10.0 vol) were charged to a suitably sized flask equipped with a mechanical stirrer, dropping funnel and thermometer. The contents were stirred under nitrogen and heated to between 75 and 85° C. Water (up to 2.5 vol) was then charged to the contents until a clear solution was obtained. The contents were then cooled to between 40 and 60° C. and concentrated under vacuum at 40 to 50° C. until the reaction volume was reduced by approximately 50%. 2-Propanol (3.0 vol) was charged to the flask and the contents were concentrated at 40 to 50° C. until approximately 3.0 vol of solvent was removed. This process was then repeated twice more with 2-propanol (2×3.0 vol) and the water content was checked. The resultant slurry was then cooled to between 0 and 5° C. and stirred at this temperature for 1 to 2 hours. The contents were filtered, the filter-cake was washed with 2-propanol (2×1.0 vol) and then pulled dry on the filter for up to 24 hours. The solid was transferred to drying trays and dried under vacuum at 45 to 50° C. to constant weight to give 4-(2,6-dichlorobenzoylamino)-1H-pyrazole-3-carboxylic acid piperidin-4-ylamide methanesulphonate as an off-white solid (60.0 to 100.0% w/w).

The recrystallisation yields for the four batches ranged between 85.6% and 90.4% and the purities of the recrystallised product ranged from 99.29% to 99.39%. A second recrystallisation increased the purity still further.

The 4-(2,6-dichlorobenzoylamino)-1H-pyrazole-3-carboxylic acid piperidin-4-ylamide methanesulphonate produced by this route had a melting point (by DSC) of 379.8° C.

### Removal of Residual Boc-Protected Product of Stage 5

In some cases, when the methanesulphonate salt was dissolved in acetate buffer, a fine precipitate consisting of residual traces of the Boc-protected free base was observed. Several techniques may be used for removing or preventing the formation of the precipitate, as set out below.

### (a) Filtration

A mixture of the methanesulphonate salt in 200 mM acetate buffer was drawn from a vial into a 20 mL single-use syringe using a sterile needle, and a clinical grade 0.2 μm filter (a Sartorius Minisart sterile single use filter unit) was then attached to the syringe. The plunger of the syringe was slowly depressed and the filtrate collected in a clean, clear glass vial. The content of the vial was a clear, colourless solution of the methanesulphonate salt free of particulate matter.

### (b) Heating in Aqueous Acid

A mixture of the methanesulphonate salt and methanesulphonic acid (0.4 eq.) in water (10 vol) was heated at 100° C. for 4 hours, and then cooled to 60° C. Analysis by TLC indicates that the methanesulphonate salt was present as a single component. 2-Propanol (10 vol) was added and the mixture cooled to 40° C. The mixture was reduced in vacuo to approximately 10 volumes, then a further portion of 2-propanol added (10 vol) and the mixture again reduced to 10 volumes. This cycle was repeated a further three times. The mixture was cooled in an ice-bath and the solid formed collected by filtration, washed with 2-propanol (5 vol) and dried in vacuo to give the methanesulphonate salt as a white to off-white solid.

### (c) Organic-Aqueous Extractions

A mixture of the methanesulphonate salt and methanesulphonic acid (0.4 eq.) in water (10 vol) was heated at 100° C. for 3 hours, and then cooled to ambient temperature. To this mixture was added THF-heptane (9:1, 10 vol) and the resultant mixture stirred vigorously to give a solution. The layers were separated and the aqueous phase washed with THF-heptane (9:1, 2×10 vol) then ethyl acetate (2×10 vol). To the aqueous phase was added 2-propanol (10 vol) and the solution was reduced in vacuo to approximately 5 volumes, then a further portion of 2-propanol added (10 vol) and the mixture again reduced to 5 volumes. This cycle was repeated a further three times. The solid formed was collected by filtration, washed with 2-propanol (5 vol) and dried in vacuo to give the methanesulphonate salt as a white to off-white solid.

### (d) Chromatography

The use of chromatographic techniques may provide a route for removing non-polar impurities from the methanesulphonate salt. It is envisaged that the use of reverse-phase methods will be particularly useful.

### Example 5

### Determination of the Crystal Structure of 4-(2,6-dichlorobenzoylamino)-1H-pyrazole-3-carboxylic acid piperidin-4-ylamide methanesulphonate by X-ray Diffraction

The compound 4-(2,6-dichlorobenzoylamino)-1H-pyrazole-3-carboxylic acid piperidin-4-ylamide methanesulphonate was prepared as described in Example 1. The crystal used for the diffraction experiment was a colourless plate with dimensions 0.05×0.08×0.14 mm³ obtained by precipitation from a water solution by 2-propanol. Crystallographic data were collected at 93 K using CuKα radiation (A=1.5418 Å) from a Rigaku rotating anode RU3HR, Osmic blue confocal optics and a Rigaku Jupiter CCD detector. Images were collected in two ω scans at 2θ=15 and 90° with a detector to crystal distance of 67 mm. Data collection was controlled by CrystalClear software and images were processed and scaled by Dtrek. Due to a high absorption coefficient (μ=4.01 mm⁻¹) data had to be corrected using 4^{th} order Fourier absorption correction. It was found that the crystals belong to an orthorhombic space group Pbca (# 61) with crystal lattice parameters at 93 K a=8.90(10), b=12.44(10), c=38.49(4) Å, α=β=γ=90°. The numbers in brackets represents the deviation (s.u., standard uncertainty).

The crystals described above and the crystal structure form a further aspect of the invention.

The crystal structure was solved using direct methods implemented in SHELXS-97. Intensity data for a total of 2710 unique reflections in a resolution range from 20-0.9 Å (2.3<θ<58.87) were used in the refinement of 271 crystallographic parameters by SHELXL-97. Final statistical parameters were: wR2=0.2115 (all data), R1=0.0869 (data with I>2σ(I)) and goodness of fit S=1.264.

One molecule of protonated free base and one mesylate anion were found in the asymmetric unit. The elemental composition of the asymmetric unit was C₁₇H₂₁Cl₂N₅O₅S and the calculated density of the crystals is 1.49 Mg/m³. Hydrogen atoms were generated on geometrical grounds while the location of heteroatom bound hydrogen atoms was confirmed by inspection of Fo-Fc difference maps. The positional and thermal parameters of hydrogen atoms were constricted to ride on corresponding non-hydrogen atoms. The thermal motion of non-hydrogen atoms was modelled by anisotropical thermal factors (see FIG. 1).

The crystal structure contains one intramolecular (N15H . . . O7 2.690 Å) and five intermolecular hydrogen bonds (see packing figure FIG. 2). Three of them link the protonated piperidine nitrogen with two mesylate anions. The first mesylate anion is linked through a single H-bond N12H12A . . . O2M 2.771 Å, while the second is involved in a bifurcated H-bond with interactions N12H12B . . . O1M 2.864 Å and N12H12B . . . O2M 3.057 Å. The remaining mesylate oxygen O3M is involved in a hydrogen bond N8H8 . . . O3M 2.928 Å. Neighbouring protonated free base molecules are linked together by a H-bond N15H15 . . . O7 2.876 Å, as well as by relatively long contact N15H15 . . . N2 3.562 Å and stacking of phenyl and pyrazole rings. These interactions are propagated infinitely along the b axis. Crystal packing contains 2D layers (in the ab plane) of mesylate anions sandwiched by an extensive network of charged H-bonds with two layers of protonated free base cations. The compact 2D sandwich layers are joined together along the c axis by stacking of phenyl rings and involving chlorine . . . phenyl interaction with Cl2 . . . C18 3.341 Å.

A graphical representation of the structure generated by the X-ray diffraction study is provided in FIG. 2.

The coordinates for the atoms making up the structure of the 4-(2,6-dichlorobenzoylamino)-1H-pyrazole-3-carboxylic acid piperidin-4-ylamide methanesulphonate are as set out in Table 2.

```
TABLE 2
space group: Pbca
unit cell at 93K with a, b & c having 5% s.u.:
a = 8.9
b = 12.4
c = 38.5
alpha = beta = gamma = 90
Coordinates in cif format:
loop_{—}
  _atom_site_label
  _atom_site_type_symbol
  _atom_site_fract_x
  _atom_site_fract_y
  _atom_site_fract_z
  _atom_site_U_iso_or_equiv
  _atom_site_adp_type
  _atom_site_occupancy
  _atom_site_symmetry_multiplicity
  _atom_site_calc_flag
  _atom_site_refinement_flags
  _atom_site_disorder_assembly
  _atom_site_disorder_group
S1M S 0.13517(17) 0.18539(13) 0.03193(5) 0.0286(5) Uani 1 1 d . . .
O1M O 0.1193(5) 0.2208(3) −0.00409(14) 0.0326(13) Uani 1 1 d . . .
O2M O 0.1551(5) 0.0681(3) 0.03330(13) 0.0331(13) Uani 1 1 d . . .
O3M O 0.0151(5) 0.2217(4) 0.05453(14) 0.0368(13) Uani 1 1 d . . .
C4M C 0.3036(8) 0.2420(6) 0.0475(2) 0.0355(19) Uani 1 1 d . . .
H4M1 H 0.3855 0.2197 0.0329 0.053 Uiso 1 1 calc R . .
H4M2 H 0.3212 0.2181 0.0708 0.053 Uiso 1 1 calc R . .
H4M3 H 0.2959 0.3189 0.0471 0.053 Uiso 1 1 calc R . .
C11 Cl 0.26158(17) 0.18137(12) 0.34133(5) 0.0325(5) Uani 1 1 d . . .
C12 Cl 0.75698(19) 0.16766(13) 0.26161(5) 0.0366(6) Uani 1 1 d . . .
N1 N 0.6277(6) −0.2419(4) 0.34903(16) 0.0276(14) Uani 1 1 d . . .
H1 H 0.5932 −0.3064 0.3484 0.033 Uiso 1 1 calc R . .
N2 N 0.7505(5) −0.2150(4) 0.36663(16) 0.0286(15) Uani 1 1 d . . .
C3 C 0.7635(7) −0.1082(5) 0.36163(19) 0.0265(17) Uani 1 1 d . . .
C4 C 0.6453(7) −0.0708(5) 0.34039(18) 0.0211(16) Uani 1 1 d . . .
C5 C 0.5616(7) −0.1594(5) 0.3322(2) 0.0277(18) Uani 1 1 d . . .
H5 H 0.4770 −0.1623 0.3181 0.033 Uiso 1 1 calc R . .
C6 C 0.8878(7) −0.0454(5) 0.3760(2) 0.0269(17) Uani 1 1 d . . .
O7 O 0.9037(5) 0.0506(3) 0.36722(14) 0.0368(13) Uani 1 1 d . . .
N8 N 0.9821(6) −0.0939(4) 0.39821(15) 0.0267(14) Uani 1 1 d . . .
H8 H 0.9626 −0.1584 0.4048 0.032 Uiso 1 1 calc R . .
C9 C 1.1147(7) −0.0417(5) 0.41139(19) 0.0253(17) Uani 1 1 d . . .
H9 H 1.1272 0.0261 0.3987 0.030 Uiso 1 1 calc R . .
C10 C 1.1019(8) −0.0148(5) 0.4502(2) 0.0330(18) Uani 1 1 d . . .
H10A H 1.0156 0.0315 0.4540 0.040 Uiso 1 1 calc R . .
H10B H 1.0866 −0.0804 0.4633 0.040 Uiso 1 1 calc R . .
C11 C 1.2429(7) 0.0412(5) 0.4630(2) 0.0349(19) Uani 1 1 d . . .
H11A H 1.2533 0.1102 0.4515 0.042 Uiso 1 1 calc R . .
H11B H 1.2355 0.0538 0.4878 0.042 Uiso 1 1 calc R . .
N12 N 1.3784(6) −0.0279(4) 0.45532(16) 0.0258(14) Uani 1 1 d . . .
H12A H 1.4618 0.0069 0.4623 0.031 Uiso 1 1 calc R . .
H12B H 1.3716 −0.0892 0.4676 0.031 Uiso 1 1 calc R . .
C13 C 1.3929(7) −0.0546(6) 0.4181(2) 0.0314(18) Uani 1 1 d . . .
H13A H 1.4790 −0.1013 0.4147 0.038 Uiso 1 1 calc R . .
H13B H 1.4098 0.0107 0.4049 0.038 Uiso 1 1 calc R . .
C14 C 1.2538(7) −0.1097(6) 0.4049(2) 0.0356(19) Uani 1 1 d . . .
H14A H 1.2425 −0.1785 0.4165 0.043 Uiso 1 1 calc R . .
H14B H 1.2639 −0.1231 0.3802 0.043 Uiso 1 1 calc R . .
N15 N 0.6215(5) 0.0371(4) 0.33108(16) 0.0256(14) Uani 1 1 d . . .
H15 H 0.6768 0.0852 0.3408 0.031 Uiso 1 1 calc R . .
C16 C 0.5183(7) 0.0697(5) 0.30805(18) 0.0213(15) Uani 1 1 d . . .
O17 O 0.4336(5) 0.0082(3) 0.29260(13) 0.0309(12) Uani 1 1 d . . .
C18 C 0.5120(6) 0.1890(5) 0.30170(17) 0.0195(15) Uani 1 1 d . . .
C19 C 0.3923(7) 0.2486(5) 0.31620(19) 0.0252(16) Uani 1 1 d . . .
C20 C 0.3785(7) 0.3569(5) 0.30904(19) 0.0267(17) Uani 1 1 d . . .
H20 H 0.2991 0.3957 0.3185 0.032 Uiso 1 1 calc R . . .
C21 C 0.4814(7) 0.4078(5) 0.28805(19) 0.0270(17) Uani 1 1 d . . .
H21 H 0.4708 0.4808 0.2834 0.032 Uiso 1 1 calc R . .
C22 C 0.6005(7) 0.3518(5) 0.27375(19) 0.0294(18) Uani 1 1 d . . .
H22 H 0.6702 0.3865 0.2597 0.035 Uiso 1 1 calc R . .
C23 C 0.6142(7) 0.2425(5) 0.2807(2) 0.0286(17) Uani 1 1 d . . .
```

### Example 6

### Preparation of 4-(2,6-dichloro-benzoylamino)-1H-pyrazole-3-carboxylic acid piperidin-4-ylamide acetic acid salt

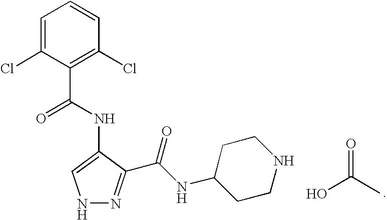

To a solution of 4-(2,6-dichloro-benzoylamino)-1H-pyrazole-3-carboxylic acid piperidin-4-ylamide hydrochloride salt (20.6 g, 50 mmol) in water (500 ml) stirring at ambient temperature was added sodium bicarbonate (4.5 g, 53.5 mmol). The mixture was stirred for 1 hour and the solid formed collected by filtration and dried in vacuo azeotroping with toluene (×3) to give the corresponding free base of 4-(2,6-dichloro-benzoylamino)-1H-pyrazole-3-carboxylic acid piperidin-4-ylamide.

¹H NMR (400 MHz, DMSO-d₆) δ 10.20 (s, 1H), 8.30 (s, 1H), 8.25 (d, 1H), 7.60-7.50 (m, 3H), 3.70 (m, 1H), 3.00 (d, 2H), 2.50 (m, 2H), 1.70 (d, 2H), 1.50 (m, 2H).

To a stirred suspension of 4-(2,6-dichloro-benzoylamino)-1H-pyrazole-3-carboxylic acid piperidin-4-ylamide (10.0 g, 26.2 mmol) in methanol (150 ml) was added glacial acetic acid (15 ml, 262 mmol) at ambient temperature. After 1 h, a clear solution was obtained which was reduced in vacuo azeotroping with toluene (×2). The residue was then triturated with acetonitrile (2×100 ml) and the solid dried in vacuo to give 4-(2,6-dichloro-benzoylamino)-1H-pyrazole-3-carboxylic acid piperidin-4-ylamide acetic acid salt (10.3 g) as a white solid.

¹H NMR (400 MHz, DMSO-d₆) δ 10.20 (s, 1H), 8.40 (d, 1H), 8.35 (s, 1H), 7.60-7.50 (m, 3H), 3.85 (m, 1H), 3.00 (d, 2H), 2.60 (t, 2H), 1.85 (s, 3H), 1.70 (d, 2H), 1.55 (m, 2H)

In the following synthetic examples, the liquid chromatography and mass spectroscopic methods used were selected from the following methods.

### Analytical LC-MS System and Method Description

In the examples, the compounds prepared were characterised by liquid chromatography and mass spectroscopy using the systems and operating conditions set out below. Where atoms with different isotopes are present, and a single mass quoted, the mass quoted for the compound is the monoisotopic mass (i.e. ³⁵Cl; ⁷⁹Br etc.). Several systems were used, as described below, and these were equipped with, and were set up to run under, closely similar operating conditions. The operating conditions used are also described below.

### Waters Platform LC-MS System:

| | |
|---|---|
| HPLC System: | Waters 2795 |
| Mass Spec Detector: | Micromass Platform LC |
| PDA Detector: | Waters 2996 PDA |

### Analytical Acidic Conditions:

| | |
|---|---|
| Eluent A: | H₂O (0.1% Formic Acid) |
| Eluent B: | CH₃CN (0.1% Formic Acid) |
| Gradient: | 5-95% eluent B over 3.5 minutes |
| Flow: | 0.8 ml/min |
| Column: | Phenomenex Synergi 4μ MAX-RP 80A, 2.0 × 50 mm |

### Analytical Long Acidic Conditions:

| | |
|---|---|
| Eluent A: | H₂O (0.1% Formic Acid) |
| Eluent B: | CH₃CN (0.1% Formic Acid) |
| Gradient: | 05-95% eluent B over 15 minutes |
| Flow: | 0.4 ml/min |
| Column: | Phenomenex Synergi 4μ MAX-RP 80A, 2.0 × 150 mm |

### Platform MS Conditions:

| | |
|---|---|
| Capillary voltage: | 3.6 kV (3.40 kV on ES negative) |
| Cone voltage: | 25 V |
| Source Temperature: | 120° C. |
| Scan Range: | 100-800 amu |
| Ionisation Mode: | ElectroSpray Positive or |
| | ElectroSpray Negative or |
| | ElectroSpray Positive & Negative |

### Waters Fractionlynx LC-MS System:

| | |
|---|---|
| HPLC System: | 2767 autosampler - 2525 binary gradient pump |
| Mass Spec Detector: | Waters ZQ |
| PDA Detector: | Waters 2996 PDA |

### Analytical Acidic Conditions:

| | |
|---|---|
| Eluent A: | H₂O (0.1% Formic Acid) |
| Eluent B: | CH₃CN (0.1% Formic Acid) |
| Gradient: | 5-95% eluent B over 4 minutes |
| Flow: | 2.0 ml/min |
| Column: | Phenomenex Synergi 4μ MAX-RP 80A, 4.6 × 50 mm |

### Fractionlynx MS Conditions:

| | |
|---|---|
| Capillary voltage: | 3.5 kV (3.2 kV on ES negative) |
| Cone voltage: | 25 V (30 V on ES negative) |
| Source Temperature: | 120° C. |
| Scan Range: | 100-800 amu |
| Ionisation Mode: | ElectroSpray Positive or |
| | ElectroSpray Negative or |
| | ElectroSpray Positive & Negative |

### Mass Directed Purification LC-MS System

Preparative LC-MS is a standard and effective method used for the purification of small organic molecules such as the compounds described herein. The methods for the liquid chromatography (LC) and mass spectrometry (MS) can be varied to provide better separation of the crude materials and improved detection of the samples by MS. Optimisation of the preparative gradient LC method will involve varying columns, volatile eluents and modifiers, and gradients. Methods are well known in the art for optimising preparative LC-MS methods and then using them to purify compounds. Such methods are described in Rosentreter U, Huber U.; Optimal fraction collecting in preparative LC/MS; J Comb Chem.; 2004; 6(2), 159-64 and Leister W, Strauss K, Wisnoski D, Zhao Z. Lindsley C., Development of a custom high-throughput preparative liquid chromatography/mass spectrometer platform for the preparative purification and analytical analysis of compound libraries; J Comb Chem.; 2003; 5(3); 322-9.

One such system for purifying compounds via preparative LC-MS is described below although a person skilled in the art will appreciate that alternative systems and methods to those described could be used. In particular, normal phase preparative LC based methods might be used in place of the reverse phase methods described here. Most preparative LC-MS systems utilise reverse phase LC and volatile acidic modifiers, since the approach is very effective for the purification of small molecules and because the eluents are compatible with positive ion electrospray mass spectrometry. Employing other chromatographic solutions e.g. normal phase LC, alternatively buffered mobile phase, basic modifiers etc as outlined in the analytical methods described above could alternatively be used to purify the compounds.

### Preparative LC-MS Systems:

### Waters Fractionlynx System:

Hardware:

### 2767 Dual Loop Autosampler/Fraction Collector

2525 preparative pump
CFO (column fluidic organiser) for column selection
RMA (Waters reagent manager) as make up pump

### Waters ZQ Mass Spectrometer

Waters 2996 Photo Diode Array detector

### Waters ZQ Mass Spectrometer

Software:

### Masslynx 4.0

Waters MS Running Conditions:

| | |
|---|---|
| Capillary voltage: | 3.5 kV (3.2 kV on ES Negative) |
| Cone voltage: | 25 V |
| Source Temperature: | 120° C. |
| Multiplier: | 500 V |
| Scan Range: | 125-800 amu |
| Ionisation Mode: | ElectroSpray Positive or |
| | ElectroSpray Negative |

### Agilent 1100 LC-MS Preparative System:

Hardware:

Autosampler: 1100 series “prepALS”
Pump: 1100 series “PrepPump” for preparative flow gradient and 1100 series “QuatPump” for pumping modifier in prep flow
UV detector: 1100 series “MWD” Multi Wavelength Detector
MS detector: 1100 series “LC-MSD VL”

### Fraction Collector: 2×“Prep-FC”

Make Up pump: “Waters RMA”

### Agilent Active Splitter

Software:

### Chemstation: Chem32

Agilent MS Running Conditions:

| | |
|---|---|
| Capillary voltage: | 4000 V (3500 V on ES Negative) |
| Fragmentor/Gain: | 150/1 |
| Drying gas flow: | 13.0 L/min |
| Gas Temperature: | 350° C. |
| Nebuliser Pressure: | 50 psig |
| Scan Range: | 125-800 amu |
| Ionisation Mode: | ElectroSpray Positive or |
| | ElectroSpray Negative |

### Chromatographic Conditions

Columns:

### 1. Low pH Chromatography:

### Phenomenex Synergy MAX-RP, 10μ, 100×21.2 mm

(alternatively used Thermo Hypersil-Keystone HyPurity Aquastar, 5μ, 100×21.2 mm for more polar compounds)

### 2. High pH Chromatography:

### Phenomenex Luna C18 (2), 10μ, 100×21.2 mm

(alternatively used Phenomenex Gemini, 5μ, 100×21.2 mm)

Eluents:

### 1. Low pH Chromatography:

Solvent A: H₂O+0.1% Formic Acid, pH˜1.5

### Solvent B: CH₃CN+0.1% Formic Acid

### 2. High pH Chromatography:

Solvent A: H₂O+10 mM NH₄HCO₃+NH₄OH, pH=9.2

### Solvent B: CH₃CN

### 3. Make Up Solvent:

MeOH+0.2% Formic Acid (for both chromatography type)

Methods:

According to the analytical trace the most appropriate preparative chromatography type was chosen. A typical routine was to run an analytical LC-MS using the type of chromatography (low or high pH) most suited for compound structure. Once the analytical trace showed good chromatography a suitable preparative method of the same type was chosen. Typical running condition for both low and high pH chromatography methods were:

Flow rate: 24 ml/min

Gradient: Generally all gradients had an initial 0.4 min step with 95% A+5% B. Then according to analytical trace a 3.6 min gradient was chosen in order to achieve good separation (e.g. from 5% to 50% B for early retaining compounds; from 35% to 80% B for middle retaining compounds and so on)

Wash: 1.2 minute wash step was performed at the end of the gradient

Re-equilibration: 2.1 minutes re-equilibration step was ran to prepare the system for the next run

Make Up flow rate: 1 ml/min

Solvent:

All compounds were usually dissolved in 100% MeOH or 100% DMSO

### Example 7

### 7A. 4-Nitro-1H-pyrazole-3-carboxylic acid methyl ester

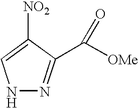

Thionyl chloride (2.90 ml, 39.8 mmol) was slowly added to a mixture of 4-nitro-3-pyrazolecarboxylic acid (5.68 g, 36.2 mmol) in MeOH (100 ml) at ambient temperature and the mixture stirred for 48 hours. The mixture was reduced in vacuo and dried through azeotrope with toluene to afford 4-nitro-1H-pyrazole-3-carboxylic acid methyl ester as a white solid.

¹H NMR (400 MHz, DMSO-d₆) δ 14.4 (s, 1H), 8.9 (s, 1H), 3.9 (s, 3H)

### 7B. 4-Amino-1H-pyrazole-3-carboxylic acid methyl ester

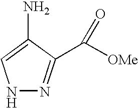

A mixture of 4-nitro-1H-pyrazole-3-carboxylic acid methyl ester and 10% Pd/C in EtOH was stirred under an atmosphere of hydrogen for 20 hours. The mixture was filtered through a plug of Celite, reduced in vacuo and dried through azeotrope with toluene to afford 4-amino-1H-pyrazole-3-carboxylic acid methyl ester.

¹H NMR (400 MHz, MeOD) δ 7.2 (s, 1H), 3.9 (s, 3H)

### 7C. 4-(2,6-dichloro-benzoylamino)-1H-pyrazole-3-carboxylic acid

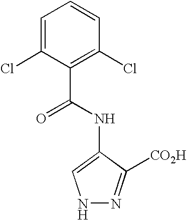

2,6-dichlorobenzoyl chloride (8.2 g; 39.05 mmol) was added cautiously to a solution of 4-amino-1H-pyrazole-3-carboxylic acid methyl ester (5 g; 35.5 mmol) and triethylamine (5.95 ml; 42.6 mmol) in dioxane (50 ml) then stirred at room temperature for 5 hours. The reaction mixture was filtered and the filtrate treated with methanol (50 ml) and 2M sodium hydroxide solution (100 ml), heated at 50° C. for 4 hours, and then evaporated. 100 ml of water was added to the residue then acidified with concentrated hydrochloric acid. The solid was collected by filtration, washed with water (100 ml) and sucked dry to give 10.05 g of 4-(2,6-dichloro-benzoylamino)-1H-pyrazole-3-carboxylic acid as a pale violet solid. (LC/MS: Rₜ 2.26, [M+H]⁺ 300/302).

### 7D. 4-{[4-(2,6-dichloro-benzoylamino)-1H-pyrazole-3-carbonyl]-amino}-piperidine-1-carboxylic acid tert-butyl ester

A mixture of 4-(2,6-dichloro-benzoylamino)-1H-pyrazole-3-carboxylic acid (6.5 g, 21.6 mmol), 4-amino-1-BOC-piperidine (4.76 g, 23.8 mmol), EDC (5.0 g, 25.9 mmol) and HOBt (3.5 g, 25.9 mmol) in DMF (75 ml) was stirred at room temperature for 20 hours. The reaction mixture was reduced in vacuo and the residue partitioned between ethyl acetate (100 ml) and saturated aqueous sodium bicarbonate solution (100 ml). The organic layer was washed with brine, dried (MgSO₄) and reduced in vacuo. The residue was taken up in 5% MeOH-DCM (˜30 ml). The insoluble material was collected by filtration and, washed with DCM and dried in vacuo to give 4-{[4-(2,6-dichloro-benzoylamino)-1H-pyrazole-3-carbonyl]-amino}-piperidine-1-carboxylic acid tert-butyl ester (5.38 g) as a white solid. The filtrate was reduced in vacuo and the residue purified by column chromatography using gradient elution 1:2 EtOAc/hexane to EtOAc to give further 4-{[4-(2,6-dichloro-benzoylamino)-1H-pyrazole-3-carbonyl]-amino}-piperidine-1-carboxylic acid tert-butyl ester (2.54 g) as a white solid.

### 7E. 4-(2,6-dichloro-benzoylamino)-1H-pyrazole-3-carboxylic acid piperidin-4-ylamide hydrochloride

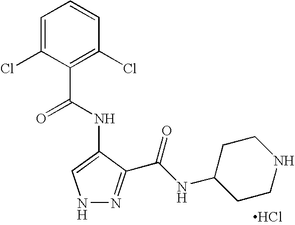

A solution of 4-{[4-(2,6-dichloro-benzoylamino)-1H-pyrazole-3-carbonyl]-amino}-piperidine-1-carboxylic acid tert-butyl ester (7.9 g) in MeOH (50 mL) and EtOAc (50 ml) was treated with sat. HCl-EtOAc (40 mL) then stirred at r.t. overnight. The product did not crystallise due to the presence of methanol, and therefore the reaction mixture was evaporated and the residue triturated with EtOAc. The resulting off white solid was collected by filtration, washed with EtOAc and sucked dry on the sinter to give 6.3 g of 4-(2,6-dichloro-benzoylamino)-1H-pyrazole-3-carboxylic acid piperidin-4-ylamide as the hydrochloride salt. (LC/MS: Rₜ 5.89, [M+H]⁺ 382/384).

### 7F. 4-(2,6-dichloro-benzoylamino)-1H-pyrazole-3-carboxylic acid (1-methanesulphonyl-piperidin-4-yl)-amide

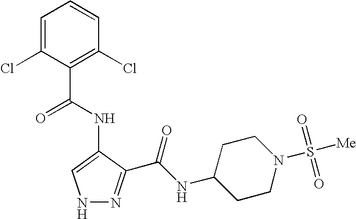

To a mixture of 4-(2,6-dichloro-benzoylamino)-1H-pyrazole-3-carboxylic acid piperidin-4-ylamide hydrochloride (1 mmol) in acetonitrile (10 ml) was added diisopropylethylamine (2.2 mmol) followed by the methanesulphonyl chloride (1 mmol). The mixture was stirred at ambient temperature for 16 hours then reduced in vacuo. The residue was partitioned between ethyl acetate and water, the layers separated and the organic portion washed with brine, dried (MgSO₄) and reduced in vacuo to give the title compound. [M+H]⁺ 460 Rₜ 2.67. LC/MS. r.t. 2.67 min; m/z 460.11

¹H NMR: (400 MHz, DMSO-d₆) δ 13.51 (s, 1H), 10.20 (s, 1H), 8.50 (d, J=8.0 Hz, 1H), 8.41 (s, 1H), 7.66-7.56 (m, 3H), 3.95-3.89 (m, 1H), 3.61 (d, J=12.0 Hz, 2H), 2.92 (s, 3H), 2.84 (t, J=12.0 Hz, 2H), 1.89-1.86 (m, 2H), 1.79-1.70 (m, 2H)

### Example 8

### 4-(2,6-dichloro-benzoylamino)-1H-pyrazole-3-carboxylic acid (1-isopropyl-sulphonyl-piperidin-4-yl)-amide

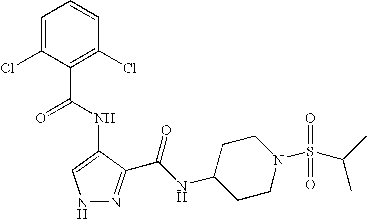

The title compound was prepared by the methods described in Example 7 but using isopropylsulphonyl chloride instead of methanesulphonyl chloride and was purified by preparative LC/MS. r.t. 2.83 min; m/z 488.22

¹H NMR: (400 MHz, DMSO-d₆) δ 13.42 (s, 1H), 10.16 (s, 1H), 8.46 (d, J=8.0 Hz, 1H), 8.35 (s, 1H), 7.60-7.51 (m, 3H), 3.92-3.87 (m, 1H), 3.65 (d, J=12.0 Hz, 2H), 3.35-3.27 (m, 1H), 2.95 (t, J=12.0 Hz, 2H), 1.80-1.76 (m, 2H), 1.66-1.59 (m, 2H), 1.22 (d, J=8.0 Hz, 6H)

### Example 9

### 4-(2,6-dichloro-benzoylamino)-1H-pyrazole-3-carboxylic acid (1-ethyl-sulphonyl-piperidin-4-yl)-amide

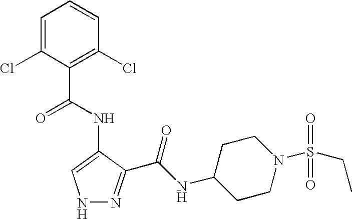

The title compound was prepared by the methods described in Example 7, but using ethylsulphonyl chloride instead of methanesulphonyl chloride, and was purified by column chromatography, eluting with P.E.-EtOAc (1:1-0:1). LC/MS. r.t. 2.74 min; m/z 474.17

¹H NMR (400 MHz, DMSO-d₆) δ 13.45 (s, 1H), 10.17 (s, 1H), 8.51 (d, J=8.0 Hz, 1H), 8.37 (s, 1H), 7.60-7.51 (m, 3H), 3.91-3.85 (m, 1H), 3.61 (d, J=12.0 Hz, 2H), 3.04 (q, J=8.0 Hz, 2H), 2.86 (t, J=12.0 Hz, 2H), 1.80-1.78 (m, 2H), 1.69-1.60 (m, 2H), 1.21 (t, J=8.0 Hz, 3H)

### Example 10

### 4-(2,6-dichloro-benzoylamino)-1H-pyrazole-3-carboxylic acid (1-propyl-sulphonyl-piperidin-4-yl)-amide

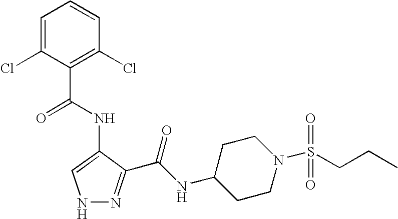

The title compound was prepared by the methods described in Example 7, but using propanesulphonyl chloride instead of methanesulphonyl chloride, and was purified by preparative LC/MS r.t. 3.11 min; m/z 488.18

¹H NMR: (400 MHz, DMSO-d₆) δ 13.42 (s, 1H), 10.15 (s, 1H), 8.46 (d, J=8.0 Hz, 1H), 8.36 (s, 1H), 7.60-7.51 (m, 3H), 3.91-3.84 (m, 1H), 3.60 (d, J=12.0 Hz, 2H), 3.00 (t, J=8.0 Hz, 2H), 2.85 (t, J=12.0 Hz, 2H), 1.82-1.78 (m, 2H), 1.72-1.62 (m, 4H), 0.99 (t, J=8.0 Hz, 3H)

### Example 11

### Synthesis of 4-(2,6-dichloro-benzoylamino)-1H-pyrazole-3-carboxylic acid (1-methanesulphonyl-piperidin-4-yl)-amide and Crystals Thereof

The compound 4-(2,6-dichloro-benzoylamino)-1H-pyrazole-3-carboxylic acid (1-methanesulphonyl-piperidin-4-yl)-amide can be prepared by the synthetic sequence illustrated in Scheme 1 above and described in more detail below.

### Stage 1: Preparation of 4-nitro-1H-pyrazole-3-carboxylic acid methyl ester

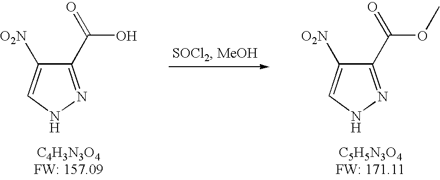

4-Nitro-1H-pyrazole-3-carboxylic acid (1.350 Kg, 8.59 Mol, 1.0 wt) and methanol (10.80 L, 8.0 vol) were charged to a flange flask equipped with a mechanical stirrer, condenser and thermometer. The suspension was cooled to 0 to 5° C. under nitrogen and thionyl chloride (0.702 L, 9.62 Mol, 0.52 vol) added at this temperature. The mixture was warmed to 15 to 25° C. over 16 to 24 hours. Reaction completion was determined by ¹H NMR analysis (d₆-DMSO). The mixture was concentrated under vacuum at 35 to 45° C. and toluene (2.70 L, 2.0 vol) charged to the residue and removed under vacuum at 35 to 45° C. The toluene azeotrope was repeated twice using toluene (2.70 L, 2.0 vol) to give 4-nitro-1H-pyrazole-3-carboxylic acid methyl ester [1.467 Kg, 99.8% th, 108.7% w/w, ¹H NMR (d₆-DMSO) concordant with structure, no entrained solvent] as an off-white solid.

### Stage 2: Preparation of 4-amino-1H-pyrazole-3-carboxylic acid methyl ester

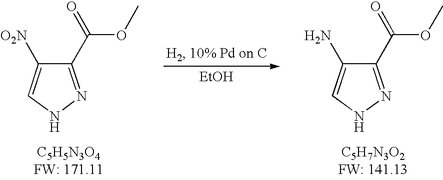

A suspension of 4-nitro-1H-pyrazole-3-carboxylic acid methyl ester (1.467 Kg, 8.57 Mol, 1.0 wt) and ethanol (14.70 L, 10.0 vol) was heated to and maintained at 30 to 35° C. until complete dissolution occurred. 10% Palladium on carbon (10% Pd/C wet paste, 0.205 Kg, 0.14 wt) was charged to a separate flask under nitrogen and a vacuum/nitrogen purge cycle performed (×3). The solution of 4-nitro-1H-pyrazole-3-carboxylic acid methyl ester in ethanol was charged to the catalyst and the vacuum/nitrogen purge cycle repeated (×3). A vacuum/hydrogen purge cycle was performed (×3) and the reaction placed under an atmosphere of hydrogen. The reaction mixture was stirred at 28 to 30° C. until deemed complete by ¹H NMR analysis (d₆-DMSO). The mixture was filtered under nitrogen and concentrated under vacuum at 35 to 45° C. to give 4-amino-1H-pyrazole-3-carboxylic acid methyl ester [, 1.184 Kg, 97.9% th, 80.7% w/w, ¹H NMR (d₆-DMSO) concordant with structure, corrected for 0.27% w/w entrained ethanol] as an off-white solid.

### Stage 3: Preparation of 4-(2,6-dichlorobenzoylamino)-1H-pyrazole-3-carboxylic acid methyl ester

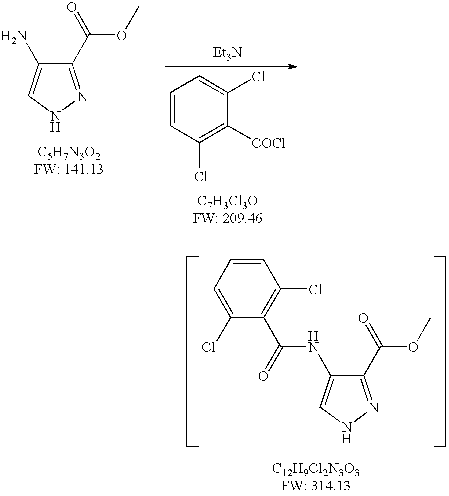

Triethylamine (1.42 L, 10.20 Mol, 1.2 vol) was added to solution of 4-amino-1H-pyrazole-3-carboxylic acid methyl ester (1.184 Kg, 8.39 Mol, 1.0 wt) in 1,4-dioxane (10.66 L, 9.0 vol) at 15 to 25° C. under nitrogen. 2,6-Dichlorobenzoyl chloride (1.33 L, 9.28 Mol, 1.12 vol) was charged at 15 to 25° C. followed by a line rinse of 1,4-dioxane (1.18 L, 1.0 vol) and the reaction mixture stirred at 15 to 25° C. for 14 to 24 hours. Reaction completion was determined by ¹H NMR analysis¹. The reaction mixture was filtered, the filter-cake washed with 1,4-dioxane (2×1.18 L, 2×1.0 vol) and the combined filtrates progressed to Stage 4 without further isolation. ¹ A sample of the reaction mixture was filtered, the filtrates dissolved in d₆-DMSO and a ¹H NMR spectrum obtained

### Stage 4: Preparation of 4-(2,6-dichlorobenzoylamino)-1H-pyrazole-3-carboxylic acid

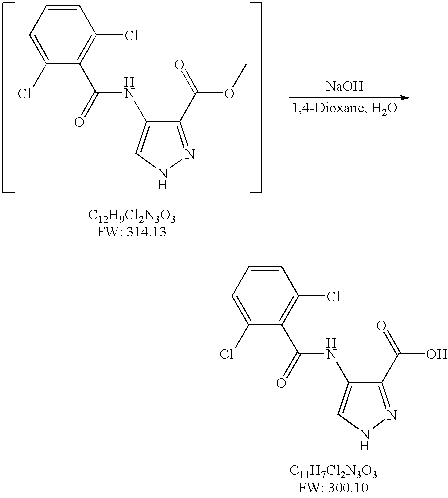

A solution of 4-(2,6-dichlorobenzoylamino)-1H-pyrazole-3-carboxylic acid methyl ester (1.308 Kg, 4.16 Mol, 1.0 wt) in 1,4-dioxane (6.47 L, 5.0 vol) was charged, in one portion, to 2M aq. sodium hydroxide solution (7.19 L, 14.38 Mol, 5.5 vol) at 35 to 45° C. The reaction mixture was cooled to 15 to 25° C. over 14 to 24 hours. Reaction completion was determined by TLC analysis². The reaction mixture was concentrated under vacuum at 45 to 50° C. The resultant oily residue was diluted with water (11.77 L, 9.0 vol) and acidified to pH1 with conc. aq. hydrochloric acid at 15 to 30° C. The precipitate was collected by filtration, washed with water (5.88 L, 4.5 vol), pulled dry on the filter and a displacement wash with heptanes (5.88 L, 4.5 vol) added. The filter-cake was charged to a 20 L rotary evaporator flask and azeo-dried with toluene (2×5.23 L, 2×4.0 vol) to afford 4-(2,6-dichlorobenzoylamino)-1H-pyrazole-3-carboxylic acid [1.207 Kg, 96.6% th, 92.3% w/w, ¹H NMR (d₆-DMSO) concordant with structure, 98.31% by HPLC area] as a yellow solid. ² Eluant: Ethyl acetate. UV visualisation. R_{f ester} 0.5, R_{f Stage 4} 0.0

### Stage 5: Preparation of 4-{[4-(2,6-dichlorobenzoylamino)-1H-pyrazole-3-carbonyl]amino}-piperidine-1-carboxylic acid tert-butyl ester

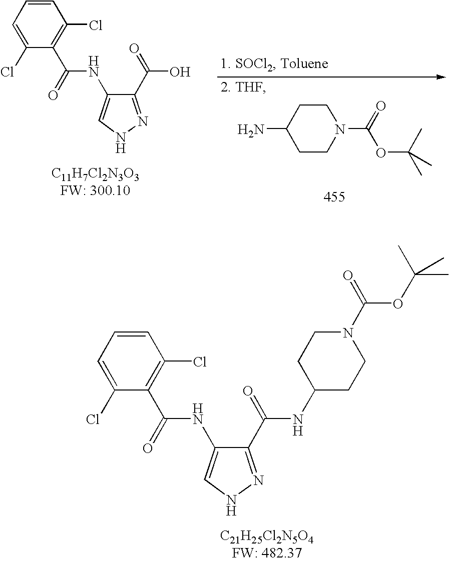

Thionyl chloride (0.25 L, 3.43 Mol, 0.3 vol) was added to a stirred suspension of 4-(2,6-dichlorobenzoylamino)-1H-pyrazole-3-carboxylic acid (0.806 Kg, 2.69 Mol, 1.0 wt) in toluene (8.00 L, 10.0 vol) under nitrogen at 16 to 25° C. The contents were then heated to and stirred at 80 to 100° C. for 16 to 24 hours. Reaction completion was determined by ¹H NMR analysis. The reaction mixture was cooled to 40 to 50° C., concentrated to dryness under vacuum at 45 to 50° C. and the residue azeo-dried with toluene (3×1.60 L, 3×2.0 vol) under vacuum at 45 to 50° C. to afford a white solid. The solid was transferred to a suitable vessel, tetrahydrofuran (4.00 L, 5.0 vol) charged, the contents stirred under nitrogen and triethylamine (0.42 L, 3.01 Mol, 0.512 vol) added at 16 to 25° C. A solution of 4-aminopiperidine-1-carboxylic acid tert-butyl ester (0.569 Kg, 2.84 Mol, 0.704 wt) in tetrahydrofuran (4.00 L, 5.0 vol) was then added to the reaction flask at 16 to 30° C. and the reaction mixture heated to and stirred at 45 to 50° C. for 2 to 16 hours. Reaction completion was determined by ¹H NMR analysis. The reaction mixture was cooled to 16 to 25° C. and quenched with water (4.00 L, 5.0 vol) and mixed heptanes (0.40 L, 0.5 vol). The contents were stirred for up to 10 minutes, the layers separated and the aqueous phase extracted with tetrahydrofuran:mixed heptanes [(9:1), 3×4.00 L, 3×5.0 vol]. The combined organic phases were washed with water (1.81 L, 2.5 vol) and concentrated under vacuum at 40 to 45° C. The residue was azeo-dried with toluene (3×4.00 L, 3×5.0 vol) to yield crude 4-{[4-(2,6-dichlorobenzoylamino)-1H-pyrazole-3-carbonyl]amino}-piperidine-1-carboxylic acid tert-butyl ester (1.257 Kg, 97.1% th, 156.0% w/w, corrected for 0.90% w/w entrained solvent). Several batches of compound were prepared in this way and the batches were combined for purification.

Crude 4-{[4-(2,6-dichlorobenzoylamino)-1H-pyrazole-3-carbonyl]amino}-piperidine-1-carboxylic acid tert-butyl ester (5.22 Mol, 1.0 wt), toluene (12.00 L, 4.87 vol) and methanol (0.30 L, 0.13 vol) were stirred under nitrogen for 3 to 18 hours at 16 to 25° C. The solid was isolated by filtration, the filter-cake washed with toluene (2×1.60 L, 2×0.7 vol) and dried under vacuum at 40 to 50° C. to yield 4-{[4-(2,6-dichlorobenzoylamino)-1H-pyrazole-3-carbonyl]amino}-piperidine-1-carboxylic acid tert-butyl ester [2.242 Kg, 86.6% th, 139.2% w/w, ¹H NMR (d₆-DMSO) concordant, 99.41% by HPLC area] as an off-white solid.

### Stage 6: Preparation of 4-(2,6-dichlorobenzoylamino)-1H-pyrazole-3-carboxylic acid piperidin-4-ylamide methanesulphonate

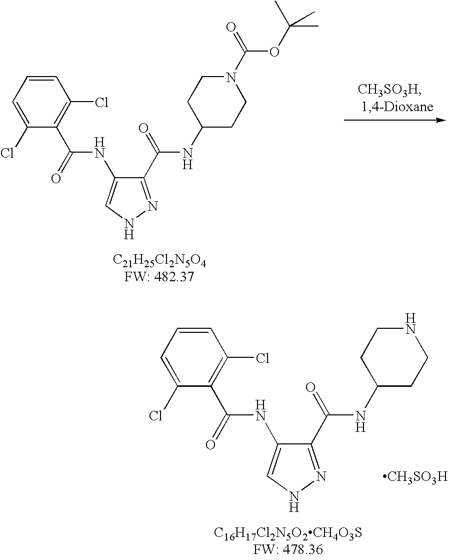

4-{[4-(2,6-Dichlorobenzoylamino)-1H-pyrazole-3-carbonyl]amino}-piperidine-1-carboxylic acid tert-butyl ester (0.561 Kg, 1.16 Mol, 1.0 wt) and 1,4-dioxane (14.00 L, 26.0 vol) were stirred under nitrogen and heated to 80 to 90° C. Methanesulphonic acid (0.30 L, 4.62 Mol, 0.54 vol) was added over 30 to 60 minutes at 80 to 90° C. and the contents heated to and maintained at 95 to 105° C. for 1 to 24 hours. Reaction completion was determined by ¹H NMR analysis. The reaction mixture was cooled to 20 to 30° C. and the resulting precipitate collected by filtration. The filter-cake was washed with propan-2-ol (2×1.10 L, 2×2.0 vol) and pulled dry on the filter for 3 to 24 hours to give 4-(2,6-dichlorobenzoylamino)-1H-pyrazole-3-carboxylic acid piperidin-4-ylamide methanesulphonate [0.558 Kg, 100.2% th, 99.4% w/w, ¹H NMR (d₆-DMSO) concordant with structure, 98.13% by HPLC area] as an off-white solid.

### Stage 7: Preparation of 4-(2,6-dichlorobenzoylamino)-1H-pyrazole-3-carboxylic acid (1-methanesulphonyl-piperidin-4-yl)-amide

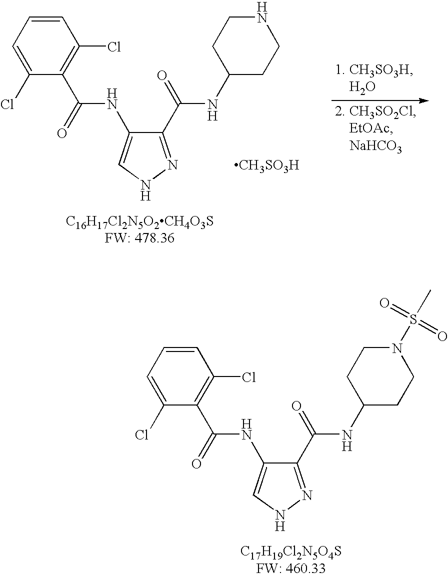

Methanesulphonic acid (0.055 L, 0.85 Mol, 0.1 vol) was added to a stirred suspension of 4-(2,6-dichlorobenzoylamino)-1H-pyrazole-3-carboxylic acid piperidin-4-ylamide methanesulphonate (0.562 Kg, 1.17 Mol, 1.0 wt) in water (5.60 L, 10.0 vol) at 15 to 40° C. The reaction mixture was heated to and stirred at 95 to 105° C. for 80 to 100 minutes. Reaction completion was determined by HPLC analysis. The mixture was cooled to 15 to 20° C., sodium hydrogen carbonate (1.224 Kg, 14.57 Mol, 2.18 wt) charged at 15 to 25° C. followed by ethyl acetate (4.20 L, 7.5 vol) and the temperature adjusted to 15 to 25° C. as necessary. Methanesulphonyl chloride (0.455 L, 5.88 Mol, 0.81 vol) was added in five aliquots over 120 to 180 minutes at 15 to 25° C. and the reaction mixture stirred for a further 30 to 45 minutes. Reaction completion was determined by HPLC analysis. The ethyl acetate was removed under vacuum at 35 to 45° C., the resulting slurry filtered, the filter-cake washed with water (0.56 L, 1.0 vol) and transferred to a suitably sized flask. Water (2.81 L, 5.0 vol) was charged and the mixture stirred for 30 to 40 minutes at 15 to 25° C. then filtered, the filter-cake washed with water (056 L, 1.0 vol) and pulled dry on the pad for 1 to 24 hours. The collected solids were dried under vacuum at 40 to 50° C. to give crude 4-(2,6-dichlorobenzoylamino)-1H-pyrazole-3-carboxylic acid (1-methanesulphonyl-piperidin-4-yl)-amide [0.490 Kg, 90.7% th, 87.2% w/w, ¹H NMR (d₆-DMSO) concordant with structure, 98.05% by HPLC area] as an off-white solid.

### Stage 8: Recrystallisation of 4-(2,6-dichlorobenzoylamino)-1H-pyrazole-3-carboxylic acid (1-methanesulphonyl-piperidin-4-yl)-amide

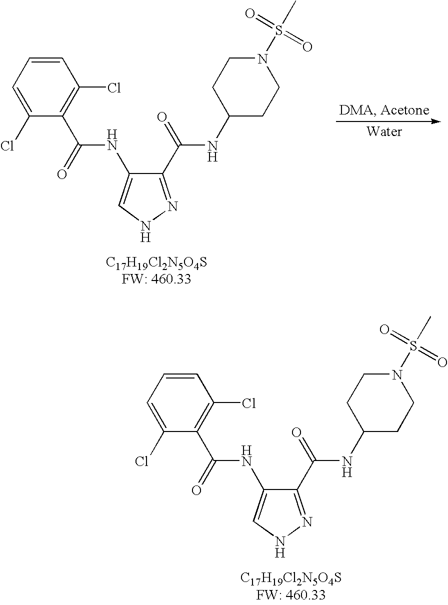

Crude 4-(2,6-dichlorobenzoylamino)-1H-pyrazole-3-carboxylic acid (1-methanesulphonyl-piperidin-4-yl)-amide (5.506 Kg, 11.96 Mol, 1.0 wt), N,N-dimethylacetamide (8.00 L, 1.5 vol) and acetone (11.00 L, 2.0 vol) were stirred under nitrogen and heated to 40 to 50° C. The resulting solution was clarified by filtration through glass microfibre paper and the filtrates heated to 60 to 80° C. Water (10.50 L, 2.0 vol) was added at 60 to 80° C. such that reflux was maintained throughout. The mixture was cooled to and aged at 15 to 25° C. for 14 to 24 hours, the crystallised solid isolated by filtration, the filter-cake washed with water (6.00 L, 1.0 vol) and transferred to a suitable vessel. Water (11.00 L, 2.0 vol) was charged, the mixture stirred for 30 to 40 minutes at 15 to 25° C. and then filtered. The filter-cake was washed with water (6.00 L, 1.0 vol) and pulled dry on the filter for at least 30 minutes. The solid was dried under vacuum at 40 to 50° C. to yield 4-(2,6-dichlorobenzoylamino)-1H-pyrazole-3-carboxylic acid (1-methanesulphonyl-piperidin-4-yl)-amide [4.530 Kg, 82.3% th, 82.3% w/w, ¹H NMR (d₆-DMSO) concordant with structure, 99.29% by HPLC area] as a white solid.

### Example 12

### Alternative Synthesis of 4-(2,6-dichloro-benzoylamino)-1H-pyrazole-3-carboxylic acid (1-methanesulphonyl-piperidin-4-yl)-amide

### Step 1: Synthesis of 4-[(4-nitro-1H-pyrazole-3-carbonyl)-amino]-piperidine-1-carboxylic acid tert-butyl ester

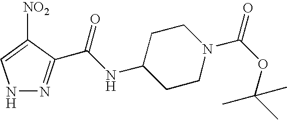

4-Nitropyrazole-3-carboxylic acid (20.0 g, 127.4 mmol) was suspended in CH₂Cl₂/DMF (99:1, 400 mL), treated cautiously with oxalyl chloride (11.6 mL, 134 mmol) and then stirred at room temperature for 16 h. The reaction mixture was evaporated then re-evaporated with toluene (×3) to give a yellow solid. The resultant acid chloride was suspended in dioxane (400 mL), treated with triethylamine (26.4 mL, 190 mmol) followed by 4-amino-1-BOC-piperidine (25.0 g, 125 mmol) and stirred at room temperature for 6 h. The reaction mixture was filtered and the solid collected stirred in water (500 mL) and then re-filtered. The solid collected was dried in vacuo, azeotroping with toluene, to give the title compound (37.6 g).

### Step 2: Synthesis of 4-nitro-1H-pyrazole-3-carboxylic acid piperidin-4-ylamide

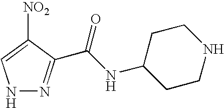

4-[(4-Nitro-1H-pyrazole-3-carbonyl)-amino]-piperidine-1-carboxylic acid tert-butyl ester (20.0 g, 59.0 mmol) was suspended in dioxane-CH₂Cl₂ (1:1, 400 ml) and treated with 4M HCl in dioxane (100 mL). The mixture was stirred at room temperature for 16 h and the solid formed collected by filtration, and dried in vacuo to give the title compound as a white solid (13.8 g).

### Step 3: Synthesis of 4-nitro-1H-pyrazole-3-carboxylic acid (1-methanesulphonyl-piperidin-4-yl)-amide

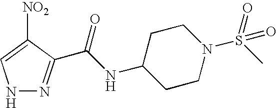

To a suspension of 4-nitro-1H-pyrazole-3-carboxylic acid piperidin-4-ylamide (13.7 g, 50.0 mmol) in dioxane-acetonitrile (1:1, 250 mL) was added triethylamine (17.4 mL, 125 mmol) followed by methanesulphonyl chloride (4.26 mL, 55.0 mmol). The mixture was stirred at 45° C. for 5 h then reduced in vacuo. To the residue was added water (500 mL), the mixture stirred for 20 min and the solid collected by filtration and dried in vacuo, azeotroping with toluene (×3), to give the title compound as an off-white solid (12.8 g)

### Step 4: Synthesis of 4-amino-1H-pyrazole-3-carboxylic acid (1-methanesulphonyl-piperidin-4-yl)-amide

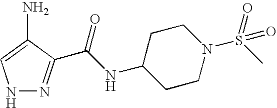

4-Nitro-1H-pyrazole-3-carboxylic acid (1-methanesulphonyl-piperidin-4-yl)-amide (5.0 g) was dissolved in DMF (30 mL), treated with 10% palladium on carbon (0.5 g) then hydrogenated at room temperature and 45 psi until the reaction was complete. The reaction mixture was filtered through Celite and reduced in vacuo. The residue was triturated with water (200 mL) and the resultant solid collected by filtration and dried in vacuo, azeotroping with toluene (×3) to give the title compound as the major product (3.5 g)

### Step 5: Synthesis of 4-(2,6-dichloro-benzoylamino)-1H-pyrazole-3-carboxylic acid (1-methanesulphonyl-piperidin-4-yl)-amide

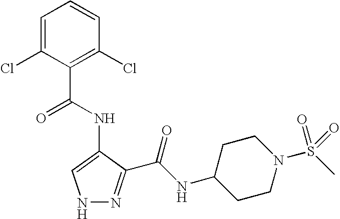

To a mixture of 4-amino-1H-pyrazole-3-carboxylic acid (1-methanesulphonyl-piperidin-4-yl)-amide (3.4 g, ˜10 mmol) and triethylamine (1.53 mL, 11 mmol) in dioxane (50 mL) at 45° C. was slowly added 2,6-dichlorobenzoyl chloride (1.4 mL, 10 mmol). The mixture was heated at 45° C. for 2 h, poured into water (250 mL) and then extracted with EtOAc (2×200 mL). The combined organic extracts were reduced in vacuo and purified by column chromatography on silica gel eluting with P.E-EtOAc (1:0-0:1). The product containing fractions were reduced in vacuo and the residue taken up in 2M aqueous NaOH-MeOH (1:1, 50 mL) and stirred at ambient temperature for 2 h. The MeOH was removed in vacuo and the mixture extracted with EtOAc. The organic portion was washed with brine, dried over MgSO₄ and reduced in vacuo. The residue was purified by hot slurry with EtOH to give the title compound as an off-white solid (2.52 g).

### Example 13

### Determination of the Crystal Structure of 4-(2,6-dichlorobenzoylamino)-1H-Pyrazole-3-carboxylic acid (1-methanesulphonyl-piperidin-4-yl)-amide by X-ray Diffraction

A crystal was obtained by evaporation of a CHCl₃ solution of the compound 4-(2,6-dichlorobenzoylamino)-1H-pyrazole-3-carboxylic acid (1-methanesulphonyl-piperidin-4-yl)-amide prepared as described in Example 6.

The crystal used for the diffraction experiment was colourless and of irregular shape with dimensions 0.15×015×0.04 mm³. Crystallographic data were collected at 104 K using CuKα radiation (λ=1.5418 Å) from a Rigaku rotating anode RU3HR, Osmic blue confocal optics, AFC9 ¼χ goniometer and a Rigaku Jupiter CCD detector. Images were collected in three ω scans at 2θ=15° and four scans at 2θ=90° with a detector to crystal distance of 67 mm. Data collection was controlled by CrystalClear software and images were processed and scaled by Dtrek. Due to a high absorption coefficient (μ=4.04 mm⁻¹) data had to be corrected using 4^{th} order Fourier absorption correction. It was found that the crystals belong to a monoclinic space group C2/c (# 15) with crystal lattice parameters a=9.15, b=31.32, c=7.93 Å, β=113.3°, α=γ=90°. One short room temperature scan was taken to check crystal lattice parameters and symmetry. It was found that symmetry is the same as at 104 K and crystal lattice parameters are similar (room temperature a=9.19, b=31.31, c=8.09 Å, β=115.2°). The unit cell dimensions a, b & c have a deviation (s.u., standard uncertainty) of 5%.

The crystal structure was solved using direct methods implemented in SHELXS-97. Intensity data for a total of 2682 unique reflections in a resolution range from 15.67-0.84 Å (2.82<θ<66.54) were used in the refinement of 263 crystallographic parameters by SHELXL-97. Final statistical parameters were: wR2=0.1749 (all data), R_{F}=0.0663 (data with I>2σ(I)) and goodness of fit S=1.035.

Only one molecule of free base was found in the asymmetric unit. The elemental composition of the asymmetric unit was C₁₇H₁₉Cl₂N₅O₄S and the calculated density of the crystals is 1.47 Mg/m³. Hydrogen atoms were generated on geometrical grounds while the location of heteroatom bound hydrogen atoms was confirmed by inspection of Fo-Fc difference maps. The positional and thermal parameters of hydrogen atoms were constricted to ride on corresponding non-hydrogen atoms. The thermal motion of non-hydrogen atoms was modelled by anisotropic thermal factors (see FIG. 3).

The crystal structure contains one intramolecular (N6-H . . . O14 2.812 Å) and one intermolecular hydrogen bond (see FIG. 4). The molecules are linked together into chains by intermolecular H-bond N1-H . . . O22 2.845 Å. Dichlorophenyl moieties from different chains stack together forming compact 3D packing.

A thermal ellipsoid representation of the structure generated by the X-ray diffraction study is provided in FIG. 3 and packing diagram is in FIG. 4.

The coordinates for the atoms making up the structure of the free base of 4-(2,6-dichlorobenzoylamino)-1H-pyrazole-3-carboxylic acid (1-methanesulphonyl-piperidin-4-yl)-amide are as set out in cif format in Table 1 below.

TABLE 1

space group: C2/c (# 15)
unit cell at 104K with a, b & c having 5% s.u.:
a = 9.150
b = 31.320
c = 7.930
alpha = gamma = 90.00
beta = 113.30
loop_{—}
_atom_site_label
_atom_site_type_symbol
_atom_site_fract_x
_atom_site_fract_y
_atom_site_fract_z
_atom_site_U_iso_or_equiv
_atom_site_adp_type
_atom_site_occupancy
_atom_site_symmetry_multiplicity
_atom_site_calc_flag
_atom_site_refinement_flags
_atom_site_disorder_assembly
_atom_site_disorder_group
Cl1 Cl 1.55055(16) 0.20997(4) 1.6202(2) 0.0376(4) Uani 1 1 d . . .
Cl2 Cl 0.97743(17) 0.20548(4) 1.6837(3) 0.0447(5) Uani 1 1 d . . .
S1 S 0.57041(12) 0.07771(3) 0.25572(15) 0.0212(3) Uani 1 1 d . . .
O7 O 1.3597(5) 0.14890(12) 1.8380(5) 0.0376(10) Uani 1 1 d . . .
O14 O 1.0227(4) 0.12633(10) 1.1610(5) 0.0266(8) Uani 1 1 d . . .
O22 O 0.4600(4) 0.04232(10) 0.1911(5) 0.0285(9) Uani 1 1 d . . .
O23 O 0.6695(4) 0.08741(13) 0.1578(5) 0.0282(9) Uani 1 1 d . . .
N1 N 1.2370(5) 0.02604(12) 1.5929(6) 0.0215(9) Uani 1 1 d . . .
H1 H 1.2665 0.0019 1.6538 0.026 Uiso 1 1 calc . . .
N2 N 1.1481(5) 0.02788(12) 1.4095(6) 0.0241(10) Uani 1 1 d . . .
N6 N 1.2053(5) 0.13987(12) 1.5365(6) 0.0226(9) Uani 1 1 d . . .
H6 H 1.1513 0.1533 1.4330 0.027 Uiso 1 1 calc . . .
N15 N 0.9606(5) 0.05870(11) 1.0508(6) 0.0192(9) Uani 1 1 d . . .
H15 H 0.9804 0.0313 1.0720 0.023 Uiso 1 1 calc . . .
N19 N 0.6881(4) 0.06785(12) 0.4705(5) 0.0185(9) Uani 1 1 d . . .
C3 C 1.1279(5) 0.06988(14) 1.3718(7) 0.0196(10) Uani 1 1 d . . .
C4 C 1.2051(5) 0.09437(14) 1.5332(7) 0.0210(10) Uani 1 1 d . . .
C5 C 1.2765(6) 0.06537(16) 1.6738(8) 0.0240(11) Uani 1 1 d . . .
H5 H 1.3393 0.0714 1.7992 0.029 Uiso 1 1 calc . . .
C7 C 1.2811(6) 0.16340(14) 1.6846(7) 0.0243(11) Uani 1 1 d . . .
C8 C 1.2638(7) 0.21135(14) 1.6550(8) 0.0239(11) Uani 1 1 d . . .
C9 C 1.3834(6) 0.23627(16) 1.6278(7) 0.0260(11) Uani 1 1 d . . .
C10 C 1.3723(7) 0.27967(18) 1.6094(8) 0.0331(13) Uani 1 1 d . . .
H10 H 1.4564 0.2955 1.5978 0.040 Uiso 1 1 calc . . .
C11 C 1.2352(7) 0.30098(16) 1.6076(8) 0.0333(14) Uani 1 1 d . . .
H11 H 1.2266 0.3311 1.5928 0.040 Uiso 1 1 calc . . .
C12 C 1.1136(7) 0.27794(18) 1.6273(8) 0.0354(14) Uani 1 1 d . . .
H12 H 1.0207 0.2921 1.6242 0.043 Uiso 1 1 calc . . .
C13 C 1.1291(6) 0.23383(16) 1.6518(8) 0.0321(14) Uani 1 1 d . . .
C14 C 1.0327(5) 0.08684(14) 1.1863(7) 0.0218(11) Uani 1 1 d . . .
C16 C 0.8492(5) 0.07270(14) 0.8678(7) 0.0184(10) Uani 1 1 d . . .
H16 H 0.7916 0.0985 0.8838 0.022 Uiso 1 1 calc . . .
C17 C 0.9342(5) 0.08479(14) 0.7426(7) 0.0211(11) Uani 1 1 d . . .
H17A H 0.9903 0.0595 0.7223 0.025 Uiso 1 1 calc . . .
H17B H 1.0142 0.1073 0.8019 0.025 Uiso 1 1 calc . . .
C18 C 0.8119(5) 0.10120(15) 0.5567(7) 0.0225(10) Uani 1 1 d . . .
H18A H 0.7612 0.1276 0.5760 0.027 Uiso 1 1 calc . . .
H18B H 0.8665 0.1080 0.4743 0.027 Uiso 1 1 calc . . .
C20 C 0.6048(5) 0.05454(15) 0.5920(7) 0.0242(11) Uani 1 1 d . . .
H20A H 0.5265 0.0319 0.5305 0.029 Uiso 1 1 calc . . .
H20B H 0.5466 0.0792 0.6132 0.029 Uiso 1 1 calc . . .
C21 C 0.7264(6) 0.03785(14) 0.7776(7) 0.0234(11) Uani 1 1 d . . .
H21A H 0.6712 0.0302 0.8584 0.028 Uiso 1 1 calc . . .
H21B H 0.7798 0.0120 0.7578 0.028 Uiso 1 1 calc . . .
C24 C 0.4560(6) 0.12321(16) 0.2544(8) 0.0279(12) Uani 1 1 d . . .
H24A H 0.5263 0.1479 0.2999 0.042 Uiso 1 1 calc . . .
H24B H 0.3984 0.1181 0.3338 0.042 Uiso 1 1 calc . . .
H24C H 0.3796 0.1288 0.1288 0.042 Uiso 1 1 calc . . .

### Example 14

### X-Ray Powder Diffraction (XRPD) Studies of Crystals of 4-(2,6-dichlorobenzoylamino)-1H-pyrazole-3-carboxylic acid (1-methanesulphonyl-piperidin-4-yl)-amide

Crystals of 4-(2,6-dichlorobenzoylamino)-1H-pyrazole-3-carboxylic acid (1-methanesulphonyl-piperidin-4-yl)-amide were prepared using the recrystallisation method described in Example 5 Step 8.

The crystal samples for X-ray powder diffraction (XRPD) data collection were gently ground by marble mortar and loaded into a crystallographic capillary (from Hampton Research, Quartz or Glass Type 10, 0.4 or 0.7 mm diameter). Diffraction patterns were collected at room temperature using CuKα radiation (λ=1.5418 Å) from a Rigaku rotating anode RU3HR, Osmic blue confocal optics, ¼ χ goniometer and a Rigaku HTC image plate detector. 2D Images were collected while spinning φ axis with a detector to crystal distance of 250 mm. Data collection was controlled by CrystalClear software and 2D images were converted to 1D plot (2θ vs. Intensity) by Datasqueeze (intensity averaged over the azimuthal angle 0≦X≦360° for 2θ range 3-30° in 0.01° or 0.02° steps). An in house program AstexXRPD was used for manipulation and visualisation of 1D XRPD patterns (FIG. 5).

TABLE 2
2θ, d-spacing and relative intensity of main peaks.

| 2θ/° | d/Å | I |
|---|---|---|
| 5.63 | 15.70 | 24 |
| 12.56 | 7.05 | 26 |
| 13.35 | 6.63 | 27 |
| 14.89 | 5.95 | 18 |
| 16.57 | 5.35 | 59 |
| 16.95 | 5.23 | 62 |
| 19.53 | 4.55 | 37 |
| 20.42 | 4.35 | 76 |
| 20.88 | 4.25 | 23 |
| 22.66 | 3.92 | 100 |
| 24.33 | 3.66 | 40 |
| 24.99 | 3.56 | 16 |

### Example 15

### Physicochemical Studies on 4-(2,6-dichlorobenzoylamino)-1H-pyrazole-3-carboxylic acid (1-methanesulphonyl-piperidin-4-yl)-amide

Crystals of 4-(2,6-dichlorobenzoylamino)-1H-pyrazole-3-carboxylic acid (1-methanesulphonyl-piperidin-4-yl)-amide prepared by the recrystallisation method of Example 11 Step 8 were subjected to differential scanning calorimetry studies and thermogravimetric analysis.

### Differential Scanning Calorimetry Study

Approximately 1-3 mg of sample (accurately weighed) were placed into an aluminium DSC pan and crimped using an aluminium lid to ensure a tight seal. The sample was then placed into a Pyris Diamond DSC (Perkin-Elmer) equipped with a liquid nitrogen cooling unit and allowed to equilibrate at 25° C. until a stable heat flow response was seen. A dry helium purge gas at a flow rate of 20 ml/min was used to produce an inert atmosphere and prevent oxidation of the sample during heating. The sample was then scanned from 25-400° C. at a scan rate of 200° C./min and the resulting heat flow response (mW) measured against temperature. Prior to experimental analysis the instrument was temperature and heat-flow calibrated using an indium reference standard.

A DSC scan of the compound is shown in FIG. 6.

### Thermogravimetric Analysis

Approximately 5 mg of sample (accurately weighed) was placed into a platinum TGA pan and loaded into a TGA 7 gravimetric analyser. The sample under study was then heated at a rate of 10° C./min (from ambient to 300° C.) and the resulting change in weight monitored. A dry nitrogen purge gas at a flow rate of 20 ml/min was used to produce an inert atmosphere and prevent oxidation of the sample during heating. Prior to analysis the instrument was weight calibrated using a 100 mg reference standard and temperature calibrated using an Alumel reference standard (using the Curie point transition temperature).

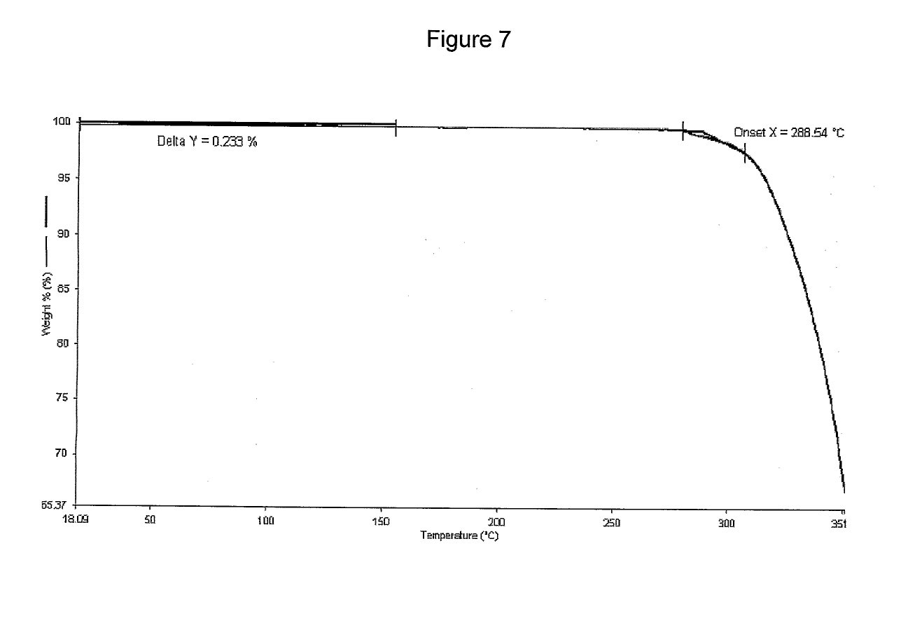

The weight loss profile of the compound is shown in FIG. 7.

### Results and Conclusions

From the resulting DSC thermograms obtained, a single defined and co-operative endothermic transition was seen onset ca. 294.5-295° C., indicative of the thermally induced melting of the crystalline lattice. No significant transitions were apparent prior to the main melting endotherm, indicating little/no loss of chemisorbed (bound) volatiles from the sample (as a result of dehydration/desolvation) as well as no detectable presence of amorphous content. This lack of a hydrated or solvated state was confirmed using TGA (FIG. 7) which showed a mass loss of approximately 0.2% up to 150° C. This suggests the existence of this drug form in the solely anhydrous crystalline state with no detectable polymorphic impurities or polymorphic transformations occurring.

The TGA plot (FIG. 7), shows a significant event at about 288° C. which occurred with an onset prior to the main melt transition, suggesting a small degree of thermally induced partial degradation of the sample prior to and during the melt. This degradation process was accelerated at temperatures greater than 300° C.

### Example 16

Vapour Sorption/Desorption Analysis of 4-(2,6-dichlorobenzoylamino)-1H-pyrazole-3-carboxylic acid (1-methanesulphonyl-piperidin-4-yl)-amide

Crystals of 4-(2,6-dichlorobenzoylamino)-1H-pyrazole-3-carboxylic acid (1-methanesulphonyl-piperidin-4-yl)-amide prepared by the recrystallisation method of Example 11 Step 8 were subjected to vapour sorption/desorption analysis in order to test for the propensity of this sample to form a hydrated state.

Approximately 20 mg of sample was placed into a wire-mesh vapour sorption balance pan and loaded into an ‘IgaSorp’ vapour sorption balance (Hiden Analytical Instruments) held at 25+/−0.1° C. The sample was then dried by maintaining a 0% humidity environment (using mass flow control apparatus) until no further weight change was recorded. Subsequently, the sample was then subjected to a ramping profile from 0-90% relative humidity (% RH) at 10% RH increments, maintaining the sample at each step until equilibration had been attained (99.5% step completion).

Upon reaching equilibration, the % RH within the apparatus was ramped to the next step and the equilibration procedure repeated. After completion of the sorption cycle, the sample was then dried using the same procedure. The weight change during the sorption/desorption cycles was then monitored, allowing for the hygroscopic nature of the sample to be determined.

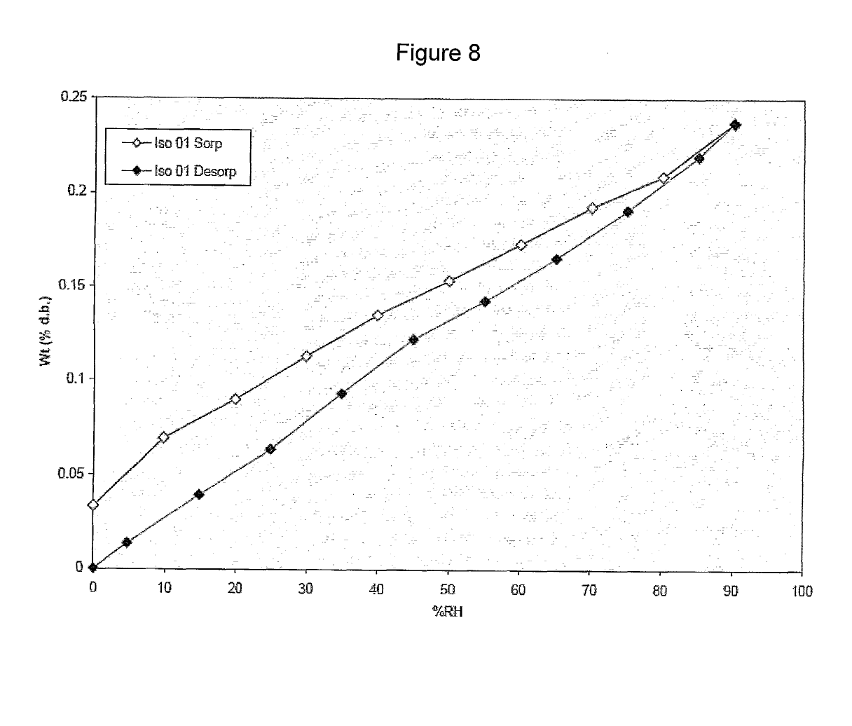

A vapour sorption/desorption profile of the compound is shown in FIG. 8.

During initial drying of the sample (at 0% RH), a weight loss of approximately 0.01% was seen, corresponding to the removal of loosely bound physi-sorbed or unbound surface adsorbed water present on the particles prior to analysis. Subsequently, increasing the relative humidity stepwise to 90% RH resulted in corresponding small incremental weight increases, totalling 0.24% upon equilibration at 90% RH. These small degrees of mass uptake seen upon storage at the varying humidities was the result of simple surface adsorption of a monolayer of water onto the particle surfaces with no true crystalline hydrate formation evident. This suggests that the compound is physically stable with regard to hygroscopicity and does not convert to the hydrated state upon storage in elevated humidity conditions.

### Biological Activity

The biological activities of the compounds of (0), (I⁰), (Ia), (Ib), (II), (III), (IV), (IVa), (Va), (Vb), (VIa), (VIb), (VII) or (VIII) and sub-groups thereof as defined in WO 2005/012256 (PCT/GB2004/003179) (and therefore herein also by dint of the incorporation of the relevant subject matter of WO 2005/012256 (PCT/GB2004/003179) by reference, see infra) and the compounds of formula (I′″) as inhibitors of CDK 4, 5 and 6 kinase are demonstrated by the examples set out below.

### Example 17

### Assay A

### Assay Procedure for CDK4

Assays for CDK4 inhibitory activity can be carried out using the proprietary 33 PanQinase® Activity Assay of Proqinase GmbH, Freiburg, Germany. The assays are performed in 96 well FlashPlates™ (PerkinElmer). In each case, the reaction cocktail (50 μl final volume) is composed of; 20 μl assay buffer (final composition 60 mM HEPES-NaOH, pH 7.5, 3 mM MgCl₂, 3 μM Na-orthovanadate, 1.2 mM DTT, 50 μg/ml PEG₂₀₀₀, 5 μl ATP solution (final concentration 1 μM [γ-33P]-ATP (approx 5×10⁵ cpm per well)), 5 μl test compound (in 10% DMSO), 10 μl substrate/10 μl enzyme solution (premixed). The final amounts of enzyme and substrate are as below.

| | | | Substrate ng/ |
|---|---|---|---|
| Kinase | Kinase ng/50 μl | Substrate | 50 μl |
| CDK4/CycD1 | 50 | Poly (Ala, Glu, Lys, | 500 |
| | | Tyr) 6:2:5:1 | |

The reaction cocktail is incubated at 30° C. for 80 minutes. The reaction is stopped with 50 μl of 2% H₃PO₄, plates are aspirated and washed twice with 200 μl 0.9% NaCl. Incorporation of ³³P is determined with a microplate scintillation counter. Background values are subtracted from the data before calculating the residual activities for each well. IC₅₀s are calculated using Prism 3.03.

### Assay B

Compounds of the invention can be tested for kinase inhibitory activity using the following protocol.

CDK4/CyclinD1 (Proqinase) is diluted to 12.5 nM in 5 mM Tris pH 7.5, 2.5 mM MgCl2, 25 μM EDTA, 2.5 mM DTT and 125 μM ATP. 10 μl of the enzyme solution is mixed with 10 μl of 100 μl biotin-KAPLSPKKAK₄ (Altabioscience, 1 mM stock-10 mg in 2,250 μl H₂O), 900 μl H₂O, 1 μl 10% triton and 35 μCi γ³³P-ATP) and added to 96 well plates along with 5 μl of various dilutions of the test compound in DMSO (up to 4%). The reaction is allowed to proceed for 2 hours before being stopped with an excess of ortho-phosphoric acid (20 μl at 2%).

γ³³P-ATP which remains unincorporated into the biotin-KAPLSPKKAK₄ is separated from phosphorylated biotin-KAPLSPKKAK₄ on a Millipore MAPH filter plate. The wells of the MAPH plate are wetted with 0.5% orthophosphoric acid, and then the results of the reaction are filtered with a Millipore vacuum filtration unit through the wells. Following filtration, the residue is washed twice with 200 μl of 0.5% orthophosphoric acid. Once the filters have dried, 20 μl of Microscint 20 scintillant is added, and then counted on a Packard Topcount for 30 seconds.

The % inhibition of the CDK4 activity is calculated and plotted in order to determine the concentration of test compound required to inhibit 50% of the CDK4 activity (IC₅₀).

### Example 18

### Further CDK Assays

Kinases are diluted to a 10× working stock in 20 mM MOPS pH 7.0, 1 mM EDTA, 0.1% γ-mercaptoethanol, 0.01% Brij-35, 5% glycerol, 1 mg/ml BSA. One unit equals the incorporation of 1 nmol of phosphate per minute into 0.1 mg/ml histone H1, or CDK7 substrate peptide at 30° C. with a final ATP concentration of 100 uM.

The substrate for all the CDK assays (except CDK7) is histone H1, diluted to 10× working stock in 20 mM MOPS pH 7.4 prior to use. The substrate for CDK7 is a specific peptide obtained from Upstate diluted to 10× working stock in deionised water.

Assay Procedure for CDK1/cyclinB, CDK2/cyclinA, CDK2/cyclinE, CDK3/cyclinE, CDK5/p35, CDK6/cyclinD3:

In a final reaction volume of 25 μl, the enzyme (5-10 mU) is incubated with 8 mM MOPS pH 7.0, 0.2 mM EDTA, 0.1 mg/ml histone H1, 10 mM MgAcetate and [γ-³³P-ATP] (specific activity approx 500 cpm/μmol, concentration as required). The reaction is initiated by the addition of Mg²⁺ [γ-³³P-ATP]. After incubation for 40 minutes at room temperature the reaction is stopped by the addition of 5 μl of a 3% phosphoric acid solution. 10 ml of the reaction is spotted onto a P30 filter mat and washed 3 times for 5 minutes in 75 mM phosphoric acid and once in methanol prior to drying and counting.

### Example 19

### Alternative CDK5 Assay

In a 96-well polypropylene plate add 5 μl of 5× test compound (in 12.5% DMSO) per well. Prepare an assay mix of 2.5 μM biotinylated histone H1 peptide (Bachem), 2.5 mM DTT in 1× assay buffer (10× assay buffer contains 250 mM Tris-HCl pH7.5, 2.5 mM MgCl2, 0.025 Brij-35, 0.1 mg/ml BSA. Add 10 μl of assay mix per well. Prepare the Cdk5/p35 enzyme (Upstate) at 0.625 nM in 1× assay buffer with 37.5 μM ATP on ice. Add 10 μl per well to start the reaction, seal with sealing film and incubate for 30 minutes at room temperature on a plate shaker. Stop the reaction by the addition of 100 μl stop buffer (1× Blocker BSA-Pierce, 0.05% surfact-AMPS-20, 100 mM EDTA). Shake plate for 1 minute. Transfer 100 μl of the stopped reaction to a black Neutravidin coated plate. Shake at room temperature for 30 minutes. Wash plate 5× with 200 μl TBS-Tween. Add 100 μl of anti-phospho cdk1-substrate antibody (Calbiochem) at 1:1500 in 1×DELFIA assay buffer (Perkin Elmer) to each well. Shake for 1 hour at room temperature. Wash plate 5× with 200 μl TBS-Tween. Add 100 μl of Eu-labelled anti-rabbit IgG at 1:300 in 1×DELFIA assay buffer per well. Shake for 1 hour at room temperature. Wash plate 5× with 200 μl TBS-Tween. Add 100 μl DELFIA enhancement solution (Perkin Elmer) per well and incubate 5 minutes on a plate shaker at <900 rpm. Read using TRF enabled fluorimeter at 335ex/620em.

The compounds of Examples 6 and 12 have IC⁵⁰ values of less than 0.1 micromolar in the above assay.

### Example 20

### Pharmaceutical Formulations

### (i) Tablet Formulation

A tablet composition containing a compound of the formulae (0) or (I′″) or an acid addition salt thereof as defined herein is prepared by mixing 50 mg of the compound or its salt with 197 mg of lactose (BP) as diluent, and 3 mg magnesium stearate as a lubricant and compressing to form a tablet in known manner.

### (ii) Capsule Formulation

A capsule formulation is prepared by mixing 100 mg of a compound of the formula (0) or (I′″) with 100 mg lactose and filling the resulting mixture into standard opaque hard gelatin capsules.

(iii) Injectable Formulation I

A parenteral composition for administration by injection can be prepared by dissolving a compound of the formula (0) (e.g. in a salt form) in water containing 10% propylene glycol to give a concentration of active compound of 1.5% by weight. The solution is then sterilised by filtration, filled into an ampoule and sealed.

### (iv) Injectable Formulation II

A parenteral composition for injection is prepared by dissolving in water a compound of the formula (0) (e.g. in salt form) (2 mg/ml) and mannitol (50 mg/ml), sterile filtering the solution and filling into sealable 1 ml vials or ampoules.

### (v) Injectable Formulation III

A formulation for i.v. delivery by injection or infusion can be prepared by dissolving the compound of formula (0) (e.g. in a salt form) in water at 20 mg/ml. The vial is then sealed and sterilised by autoclaving.

### (vi) Injectable Formulation IV

A formulation for i.v. delivery by injection or infusion can be prepared by dissolving the compound of formula (0) (e.g. in a salt form) in water containing a buffer (e.g. 0.2 M acetate pH 4.6) at 20 mg/ml. The vial is then sealed and sterilised by autoclaving.

(vii) Subcutaneous Injection Formulation

A composition for sub-cutaneous administration is prepared by mixing a compound of the formula (0) or (I′″) with pharmaceutical grade corn oil to give a concentration of 5 mg/ml. The composition is sterilised and filled into a suitable container.

(viii) Lyophilised Formulation

Aliquots of formulated compound of formula (I′″) or (0) or an acid addition salt thereof are put into 50 mL vials and lyophilized. During lyophilisation, the compositions are frozen using a one-step freezing protocol at (−45° C.). The temperature is raised to −10° C. for annealing, then lowered to freezing at −45° C., followed by primary drying at +25° C. for approximately 3400 minutes, followed by a secondary drying with increased steps if temperature to 50° C. The pressure during primary and secondary drying is set at 80 millitor.

(ix) Concentrate for Use in i.v. Administration

An aqueous buffered solution is prepared by dissolving 4-(2,6-dichlorobenzoylamino)-1H-pyrazole-3-carboxylic acid piperidin-4-ylamide methanesulphonate at a concentration of 20 mg/ml in a 0.2M sodium acetate/acetic acid buffer at a pH of 4.6.

The buffered solution is filled, with filtration to remove particulate matter, into a container (such as class 1 glass vials) which is then sealed (e.g. by means of a Florotec stopper) and secured (e.g. with an aluminium crimp). If the compound and formulation are sufficiently stable, the formulation is sterilised by autoclaving at 121° C. for a suitable period of time. If the formulation is not stable to autoclaving, it can be sterilised using a suitable filter and filled under sterile conditions into sterile vials. For intravenous administration, the solution can be dosed as is, or can be injected into an infusion bag (containing a pharmaceutically acceptable excipient, such as 0.9% saline or 5% dextrose), before administration.

### (x) Solid Solution Formulation

The compound 4-(2,6-dichlorobenzoylamino)-1H-pyrazole-3-carboxylic acid (1-methanesulphonyl-piperidin-4-yl)-amide and PVP are dissolved in dichloromethane/ethanol (1:1) at a concentration of 5 to 50% (for example 16 or 20%) and the solution is spray dried using conditions corresponding to those set out in the table below. The data given in the table include the concentration of the compound of Example 1, the inlet and outlet temperatures of the spray drier, the total yield of spray dried solid, the concentration of the compound of Example 1 in the spray dried solid (assay), and the particle size distribution (P.S.D.) of the particles making up the spray dried solid.

| | conc sol. | temp. | temp. | % | assay | PSD |
|---|---|---|---|---|---|---|
| Batch | w/vol | inlet | outlet | yield | (mg/g) | (range) (μm) |
| BR1A | 16% | 140° C. | 80° C. | 87.00 | 246.41 | 4.46-52.76 |
| BR1B | 16% | 180° C. | 80° C. | 97.00 | 246.65 | 14.83-91.70 |
| BR2A | 20% | 160° C. | 80° C. | 99.40 | 239.60 | 15.86-85.01 |
| BR3A | 20% | 180° C. | 100° C. | 79.50 | 246.64 | 15.09-91.84 |

The solid solution of the compound and PVP can either be filled directly into hard gelatin or HPMC (hydroxypropylmethyl cellulose) capsules, or be mixed with pharmaceutically acceptable excipients such as bulking agents, glidants or dispersants. The capsules could contain the compound in amounts of between 2 mg and 200 mg, for example 10, 20 and 80 mg. Alternatively, the capsules could contain 40 mg of compound.

### Example 21

### Pharmaceutical Formulations Containing a Solid Dispersion of 4-(2,6-dichloro-benzoylamino)-1H-pyrazole-3-carboxylic acid (1-methanesulphonyl-piperidin-4-yl)-amide in Polyvinylpyrrolidone (PVP)

This example describes the preparation of granule compositions containing a spray dried solid dispersion of 4-(2,6-dichloro-benzoylamino)-1H-pyrazole-3-carboxylic acid (1-methanesulphonyl-piperidin-4-yl)-amide and the K30 grade of polyvinylpyrrolidone (Kollidon K30) available from BASF ChemTrade GmbH of Burgbernheim, Germany). The molecular weight of the PVP is in the range 44,000-54,000.

The solid dispersion was prepared by dissolving 4-(2,6-dichloro-benzoylamino)-1H-pyrazole-3-carboxylic acid (1-methanesulphonyl-piperidin-4-yl)-amide in a 1:1 (v/v) mixture of ethanol and dichloromethane to give a concentration of the compound of 50 mg/mL, and then adding PVP K30 in a ratio of compound to PVP of 1:3.

The solute was then spray dried in a Niro Mobile Minor 2000 spray dryer. The powder collected from the spray dryer was dried under vacuum.

The spray drying conditions were as follows:

| Nozzle internal diameter (ID): | 1 mm |
|---|---|
| Tubing ID: | 3 mm |
| Inlet temperature: | 180° C. |
| Exhaust temperature: | 85° C. |
| Atomisation pressure: | 1.0 bar |
| Process gas flow: | 3.2 mbar (83 kg/h of nitrogen) |
| Process gas: | nitrogen |
| Solution dry weight (compound + PVP): | 1980 g |
| Flow rate: | 123 g/min |
| Yield: | 84.85% |

The particle size distribution of the spray dried solid dispersion, following drying, was measured using a laser diffraction apparatus and gave D10, D50 and D90 figures as follows:

| | |
|---|---|
| D10/μm | 17.53 |
| D50/μm | 49.08 |
| D90/μm | 93.26 |

In the following example, the solid dispersion of 4-(2,6-dichloro-benzoylamino)-1H-pyrazole-3-carboxylic acid (1-methanesulphonyl-piperidin-4-yl)-amide in PVP is referred to as “Compound of formula (I)/PVP”.

The following materials were blended for 30 seconds in a high shear mixer:—

| | |
|---|---|
| Dicalcium phosphate (Emcompress ™) | 32.8 g |
| Silicified microcrystalline cellulose (ProSolv HD90 ™) | 10.9 g |
| Compound of formula (I)/PVP | 35.2 g |
| Croscarmellose sodium (Ac-Di-Sol ™) | 11.1 g |

The powder blend was then compressed using a Freund roller compactor. The following settings were required to produce a ribbon:—

| | |
|---|---|
| Feed speed: | 60 rpm |
| Roller speed: | 2 rpm |
| Roller pressure: | 180 kg f/cm² |

The ribbon of compressed powder was ground through a 710 μm sieve and the resulting granules were collected in a suitable container. An aliquot of the granule mass (9.0 g) was mixed with a further aliquot of Ac-Di-Sol (1.0 g). The quantity of the granule mass that could be filled into size 0 capsules was determined (both flush-filled and tightly packed). Results are summarised below.

Capsule fill weight

| Flush-filled | Tightly packed |
|---|---|
| 282 mg (24.8 mg compound) | 431 mg (37.9 mg) |

### Disintegration Tests

For rapid release oral formulations, it is desirable that disintegration of the dosage form and release of the active ingredient should occur within 15 minutes. The capsule formulation described was therefore subjected to disintegration testing using a standard tablet/capsule disintegration apparatus (European Pharmacopoeia, 4^{th} Edition). Distilled water was used as the disintegration medium. The volume of the disintegration medium was 800 mL and the temperature was maintained at 37° C. (+/−1° C.). The assessment of dispersion/dissolution behaviour of the formulation was made by observation alone. The disintegration times are set out in the table below.

| Quantity of Compound of | |
|---|---|
| formula (I) per capsule | |
| (mg) | Disintegration time (min) |
| 24.8 (flush-filled) | 4 |
| 37.9 (tightly packed) | 5 |

### Dissolution Testing

The rate of dissolution of the capsule formulation was compared with the rate of dissolution of (1) the non-encapsulated solid dispersion of PVP and the compound of formula (I) containing no further excipients and (2) the solid dispersion (1) packed tightly into a size 0 capsule, and (3) the formulated sample.

The dissolution testing was conducted using the paddle apparatus as described in the European Pharmacopoeia, 4^{th} Edition.

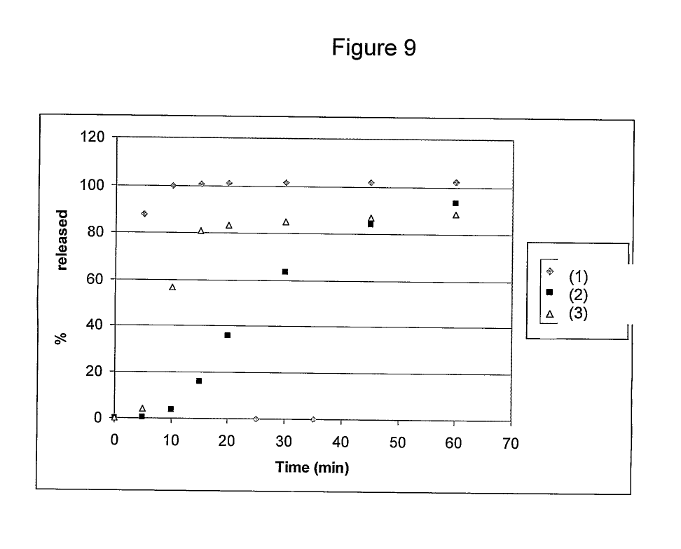

The results of the dissolution studies are shown in FIG. 9, where (1) indicates the non-encapsulated solid dispersion of PVP and the compound of formula (I) containing no further excipients; (2) indicates the solid dispersion (1) packed tightly into a size 0 capsule and (3) indicates the formulated sample.

The results show that dissolution of the non-encapsulated solid dispersion was quicker than the dissolution of the capsule sample. In the tightly packed encapsulated sample, the PVP is probably binding the particles together, thus retarding the release of the compound of formula (I). Interestingly, the formulated sample exhibited a much more rapid compound release profile compared with the non-formulated, encapsulated sample, which indicates that the high proportion of disintegrant in the formulation is effective in countering the binding capacity of the PVP.

### Example 23

### Methods of Testing for Pain Reducing or Pain Preventing Activity

### (I) Inflammatory Hyperalgesia Test

Mechanical hyperalgesia can be examined in a rat model of inflammatory pain. Paw withdrawal thresholds to an increasing pressure stimulus are measured by the Randal-Sellito technique using an analgesymeter (Ugo Basile, Milan), in naïve animals prior to an intraplantar injection of complete Freund's complete adjuvant (FCA) into the left hind paw. 24 h later paw withdrawal thresholds are measured again prior to (predose) and then from 10 min to 6 h following drug or vehicle administration. Reversal of hyperalgesia in the ipsilateral paw is calculated according to the formula:

$$\% reversal = \frac{postdose threshold - predose threshold}{naive threshold - predose threshold} \times 100$$

### (ii) Neuropathic Hyperalgesia Test

Mechanical hyperalgesia can be examined in a rat model of neuropathic pain induced by partial ligation of the left sciatic nerve. Approximately 14 days following surgery mechanical withdrawal thresholds of both the ligated (ipsilateral) and non-ligated (contralateral) paw are measured prior to (predose) and then from 10 min to 6 h following drug or vehicle administration. Reversal of hyperalgesia at each time point is calculated according to the formula:

$$\% reversal = \frac{\begin{matrix}ipsilateral threshold postdose - \\ ipsilateral threshold predose\end{matrix}}{\begin{matrix}contralateral threshold predose - \\ ipsilateral threshold predose\end{matrix}} \times 100$$

All experiments are carried out using groups of 6 animals. Stock concentrations of drugs are dissolved in distilled water and subsequent dilutions were made in 0.9% saline for subcutaneous administration in a volume of 4 mlkg⁻¹. All drugs are made up in plastic vials and kept in the dark.

Statistical analysis are carried out on withdrawal threshold readings (g) using ANOVA with repeated measures followed by Tukey's HSD test. Efficacy refers to the maximal reversal of hyperalgesia observed at the doses used.

(iii) Testing the Effects of Compounds of Formula (0) a Rat Model of Bone Cancer Pain

Adult female rats are given intra-tibial injections of MRMZ-1 rat mammary gland carcinoma cells (3 μl, 10⁷ cells/ml). The animals typically gradually develop mechanical hyperalgesia, mechanical allodynia (skin sensitivity to non-noxious stimuli) and hind limb sparing, beginning on day 12-14 following cell injection. A compound of formula (0) (e.g. at a dose of 10 and 30 μg/kg s.c.) is administered 3 times a week from the day of cell injection, and the extent of inhibition of hind limb sparing and mechanical allodynia is determined in comparison to vehicle-treated controls.

### Example 24

### Further Compounds According to the Invention

Examples 1 to 254 of WO2005/012256 at pages 121 to 222 are incorporated herein by reference, so that examples of the preparation of the following compounds are specifically described herein:
4-Amino-1H-pyrazole-3-carboxylic acid phenylamide
4-Acetylamino-1H-pyrazole-3-carboxylic acid (4-fluoro-phenyl)-amide
4-(2,2,2-Trifluoro-acetylamino)-1H-pyrazole-3-carboxylic acid (4-fluoro-phenyl)-amide
4-[(5-Oxo-pyrrolidine-2-carbonyl)-amino]-1H-pyrazole-3-carboxylic acid (4-fluoro-phenyl)-amide
4-Phenylacetylamino-1H-pyrazole-3-carboxylic acid (4-fluoro-phenyl)-amide
4-(2-1H-Indol-3-yl-acetylamino)-1H-pyrazole-3-carboxylic acid (4-fluoro-phenyl)-amide
4-(2-Benzenesulphonyl-acetylamino)-1H-pyrazole-3-carboxylic acid (4-fluoro-phenyl)-amide
4-[2-(5-Amino-tetrazol-1-yl)-acetylamino]-1H-pyrazole-3-carboxylic acid (4-fluoro-phenyl)-amide
N-[3-(4-Fluoro-phenylcarbamoyl)-1H-pyrazol-4-yl]-6-hydroxy-nicotinamide
4-[3-(4-Chloro-phenyl)-propionylamino]-1H-pyrazole-3-carboxylic acid (4-fluoro-phenyl)-amide
4-(3-4H-[1,2,4]Triazol-3-yl-propionylamino)-1H-pyrazole-3-carboxylic acid (4-fluoro-phenyl)-amide
4-[2-(1-Methyl-1H-indol-3-yl)-acetylamino]-1H-pyrazole-3-carboxylic acid (4-fluoro-phenyl)-amide
4-[(1-Hydroxy-cyclopropanecarbonyl)-amino]-1H-pyrazole-3-carboxylic acid (4-fluoro-phenyl)-amide
1-Acetyl-piperidine-4-carboxylic acid [3-(4-fluoro-phenylcarbamoyl)-1H-pyrazol-4-yl]-amide
4-[3-(4-Methyl-piperazin-1-yl)-propionylamino]-1H-pyrazole-3-carboxylic acid (4-fluoro-phenyl)-amide
4-(2-1H-Imidazol-4-yl-acetylamino)-1H-pyrazole-3-carboxylic acid (4-fluorophenyl)-amide
4-(3-Morpholin-4-yl-propionylamino)-1H-pyrazole-3-carboxylic acid (4-fluorophenyl)-amide
4-(3-Piperidin-1-yl-propionylamino)-1H-pyrazole-3-carboxylic acid (4-fluoro-phenyl)-amide
4-Cyclohexylamino-1H-pyrazole-3-carboxylic acid (4-fluoro-phenyl)-amide
4-Isopropylamino-1H-pyrazole-3-carboxylic acid (4-fluoro-phenyl)-amide
4-(2-Hydroxy-1-methyl-ethylamino)-1H-pyrazole-3-carboxylic acid (4-fluorophenyl)-amide
4-(1-Ethyl-propylamino)-1H-pyrazole-3-carboxylic acid (4-fluoro-phenyl)-amide
4-(3-Chloro-pyrazin-2-ylamino)-1H-pyrazole-3-carboxylic acid (4-fluoro-phenyl)-amide
4-(Pyrazin-2-ylamino)-1H-pyrazole-3-carboxylic acid (4-fluoro-phenyl)-amide
4-(2-Methoxy-benzoylamino)-1H-pyrazole-3-carboxylic acid (4-fluoro-phenyl)-amide
4-Benzoylamino-1H-pyrazole-3-carboxylic acid (4-fluoro-phenyl)-amide
4-(Cyclohexanecarbonyl-amino)-1H-pyrazole-3-carboxylic acid (4-fluoro-phenyl)-amide
4-[(1-Methyl-cyclopropanecarbonyl)-amino]-1H-pyrazole-3-carboxylic acid (4-fluoro-phenyl)-amide
4-(2-Hydroxy-acetylamino)-1H-pyrazole-3-carboxylic acid (4-fluoro-phenyl)-amide
4-(2,2-Dimethyl-propionylamino)-1H-pyrazole-3-carboxylic acid (4-fluoro-phenyl)-amide
4-(3-Hydroxy-propionylamino)-1H-pyrazole-3-carboxylic acid (4-fluoro-phenyl)-amide
4-(2-Fluoro-benzoylamino)-1H-pyrazole-3-carboxylic acid (4-fluoro-phenyl)-amide
4-(3-Fluoro-benzoylamino)-1H-pyrazole-3-carboxylic acid (4-fluoro-phenyl)-amide
4-(3-Methoxy-benzoylamino)-1H-pyrazole-3-carboxylic acid (4-fluoro-phenyl)-amide
4-(4-Nitro-benzoylamino)-1H-pyrazole-3-carboxylic acid (4-fluoro-phenyl)-amide
4-[(3-Methyl-furan-2-carbonyl)-amino]-1H-pyrazole-3-carboxylic acid (4-fluoro-phenyl)-amide
4-[(Furan-2-carbonyl)-amino]-1H-pyrazole-3-carboxylic acid (4-fluoro-phenyl)-amide
4-[(3H-Imidazole-4-carbonyl)-amino]-1H-pyrazole-3-carboxylic acid (4-fluoro-phenyl)-amide
4-(4-Fluoro-benzoylamino)-1H-pyrazole-3-carboxylic acid (4-fluoro-phenyl)-amide
4-(2,6-Difluoro-benzoylamino)-1H-pyrazole-3-carboxylic acid (4-fluoro-phenyl)-amide
4-(3-Nitro-benzoylamino)-1H-pyrazole-3-carboxylic acid (4-fluoro-phenyl)-amide
1H-Indole-3-carboxylic acid [3-(4-fluoro-phenylcarbamoyl)-1H-pyrazol-4-yl]-amide
4-(4-Hydroxymethyl-benzoylamino)-1H-pyrazole-3-carboxylic acid (4-fluoro-phenyl)-amide
4-(3-Methyl-benzoylamino)-1H-pyrazole-3-carboxylic acid (4-fluoro-phenyl)-amide
4-(2-Methyl-benzoylamino)-1H-pyrazole-3-carboxylic acid (4-fluoro-phenyl)-amide
4-(4-Methyl-benzoylamino)-1H-pyrazole-3-carboxylic acid (4-fluoro-phenyl)-amide
4-[(2-Methyl-thiophene-3-carbonyl)-amino]-1H-pyrazole-3-carboxylic acid (4-fluoro-phenyl)-amide
Quinoline-2-carboxylic acid [3-(4-fluoro-phenylcarbamoyl)-1H-pyrazol-4-yl]-amide
4-[(Thiophene-3-carbonyl)-amino]-1H-pyrazole-3-carboxylic acid (4-fluoro-phenyl)-amide
4-(2-fluoro-3-methoxy-benzoylamino)-1H-pyrazole-3-carboxylic acid (4-fluoro-phenyl)-amide
4-[2-(2-Pyrrolidin-1-yl-ethoxy)-benzoylamino]-1H-pyrazole-3-carboxylic acid 4-fluorophenylamide
4-(2,6-Difluoro-benzoylamino)-1H-pyrazole-3-carboxylic acid (1-methyl-piperidin-4-yl)-amide
4-(Cyclohexyl-methyl-amino)-1H-pyrazole-3-carboxylic acid (4-fluoro-phenyl)-amide
4-(Pyridin-2-ylamino)-1H-pyrazole-3-carboxylic acid (4-fluoro-phenyl)-amide
4-[(4-Amino-1-methyl-1H-imidazole-2-carbonyl)-amino]-1H-pyrazole-3-carboxylic acid (4-fluoro-phenyl)-amide
4-{[4-(2,6-Difluoro-benzoylamino)-1H-pyrazole-3-carbonyl]-amino}-cyclohexanecarboxylic acid
4-(2,6-difluoro-benzoylamino)-1H-pyrazole-3-carboxylic acid [5-fluoro-2-(1-methyl-piperidin-4-yloxy)-phenyl]-amide
4-(2,6-Difluoro-benzoylamino)-1H-pyrazole-3-carboxylic acid [5-fluoro-2-(2-pyrrolidin-1-yl-ethoxy)-phenyl]-amide
4-(4-Methyl-piperazin-1-yl)-1H-pyrazole-3-carboxylic acid (4-fluoro-phenyl)-amide
4-Morpholin-4-yl-1H-pyrazole-3-carboxylic acid (4-fluoro-phenyl)-amide
4-(2,4-Dichloro-phenyl)-1H-pyrazole-3-carboxylic acid 4-(4-methyl-piperazin-1-yl)-benzylamide
4-(2,4-Dichloro-phenyl)-1H-pyrazole-3-carboxylic acid 4-methylsulphamoylmethyl-benzylamide
4-Phenyl-1H-pyrazole-3-carboxylic acid amide
4-phenyl-1H-pyrazole-3-carboxylic acid phenylamide
4-Phenyl-1H-pyrazole-3-carboxylic acid 4-(4-methyl-piperazin-1-yl)-benzylamide
4-Phenyl-1H-pyrazole-3-carboxylic acid (6-methoxy-pyridin-3-yl) amide
4-(3-Benzyloxy-phenyl)-1H-pyrazole-3-carboxylic acid 4-(4-methyl-piperazin-1-yl)-benzylamide
4-(3-Hydroxy-phenyl)-1H-pyrazole-3-carboxylic acid 4-(4-methyl-piperazin-1-yl)-benzylamide
4-(5-Methyl-3H-imidazol-4-yl)-1H-pyrazole-3-carboxylic acid (4-fluoro-phenyl)-amide
4-(2,5-Dimethyl-pyrrol-1-yl)-1H-pyrazole-3-carboxylic acid (4-fluoro-phenyl)-amide
4-(3-Hydroxymethyl-phenyl)-1H-pyrazole-3-carboxylic acid phenylamide
4-(2,6-dichloro-benzoylamino)-1H-pyrazole-3-carboxylic acid piperidin-4-ylamide hydrochloride
4-Methanesulfonylamino-1H-pyrazole-3-carboxylic acid (4-fluoro-phenyl)-amide
4-(2,6-Difluoro-benzoylamino)-1H-pyrazole-3-carboxylic acid [1-(2-fluoro-ethyl)-piperidin-4-yl]-amide
4-(2,6-Dichloro-benzoylamino)-1H-pyrazole-3-carboxylic acid (6-chloro-pyridin-3-yl)-amide
4-(2,6-Dichloro-benzoylamino)-1H-pyrazole-3-carboxylic acid (6-amino-pyridin-3-yl)-amide
4-(2,6-Dichloro-benzoylamino)-1H-pyrazole-3-carboxylic acid (6-methoxy-pyridin-3-yl)-amide
4-[3-Chloro-5-(4-methyl-piperazin-1-yl)-benzoylamino]-1H-pyrazole-3-carboxylic acid cyclohexylamide
4-(2,6-Difluoro-benzoylamino)-1H-pyrazole-3-carboxylic acid [1-(2,2-difluoro-ethyl)-piperidin-4-yl]-amide
4-[3-(4-Methyl-piperazin-1-yl)-benzoylamino]-1H-pyrazole-3-carboxylic acid cyclohexylamide
4-(2,6-dichloro-benzoylamino)-1H-pyrazole-3-carboxylic acid piperidin-4-ylamide acetic acid salt
Methanesulphonic acid salt of 4-(2,6-dichloro-benzoylamino)-1H-pyrazole-3-carboxylic acid piperidin-4-ylamide

The compounds set out in the Tables below:

| Structure | LCMS |
|---|---|
| 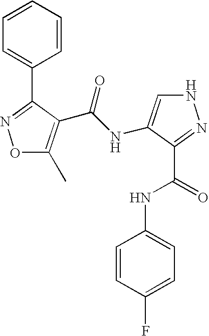 | Rₜ 3.20 min [M + H]⁺ 406.07 |
| 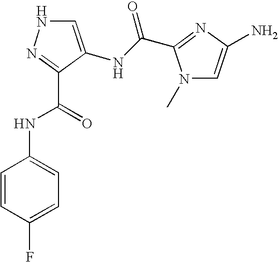 | Rₜ 2.35 min m/z 343.72 |
| 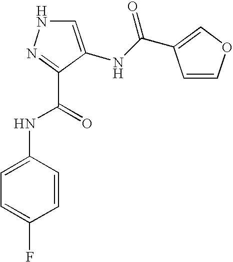 | Rₜ 3.51 min m/z 314.62 |
| 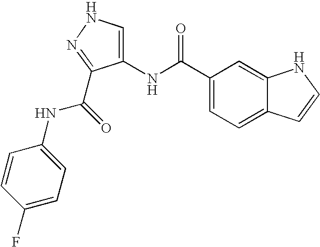 | Rₜ 3.79 min m/z 363.67 |
| 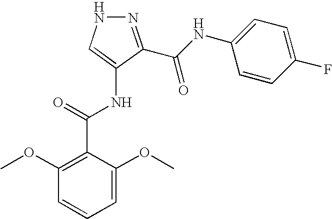 | Rₜ 3.68 min m/z 384.69 |
| 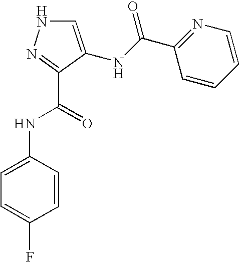 | Rₜ 3.61 min m/z 326.10 |
| 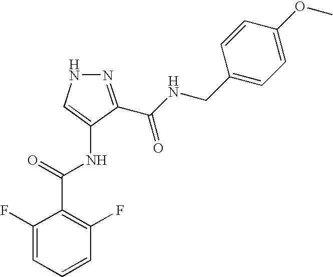 | Rₜ 3.51 min m/z 387.11 |
| 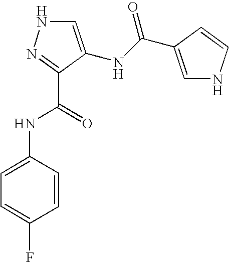 | Rₜ 3.11 min m/z 313.65 |
| 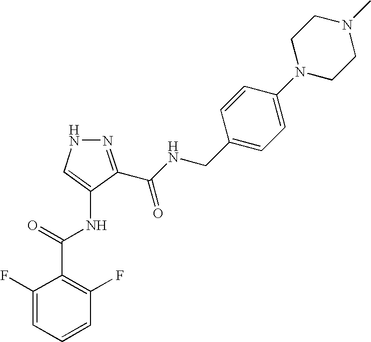 | Rₜ 2.20 min m/z 455.19 |
| 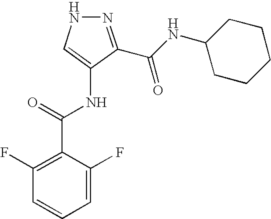 | Rₜ 3.95 min m/z 349.09 |
| 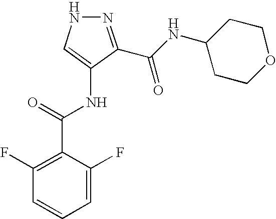 | Rₜ 2.39 min m/z 351.07 |
| 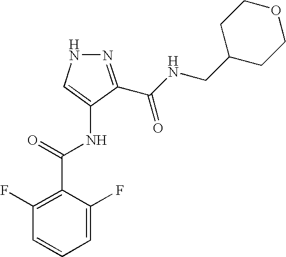 | Rₜ 2.83 min m/z 365.13 |
| 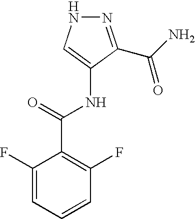 | Rₜ 2.10 min m/z 266.97 |
| 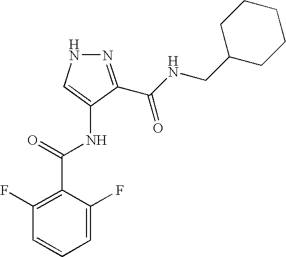 | Rₜ 3.22 min m/z 363.10 |
| 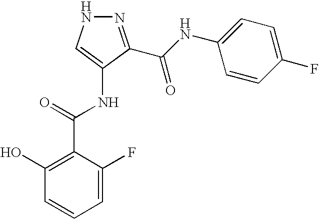 | Rₜ 4.48 min m/z 358.96 |
| 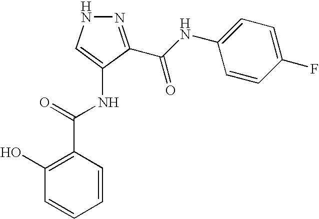 | Rₜ 3.93 min m/z 340.96 |
| 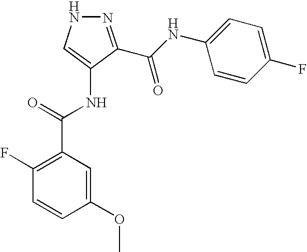 | Rₜ 4.11 min m/z 373.01 |
| 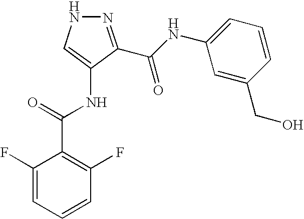 | Rₜ 2.56 min m/z 373.05 |
| 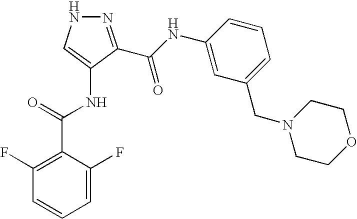 | Rₜ 1.99 min m/z 442.09 |
| 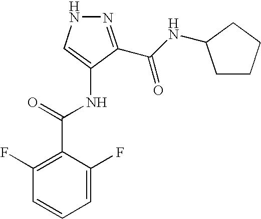 | Rₜ 3.65 min m/z 335.03 |
| 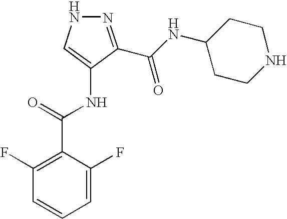 | Rₜ 1.57 min m/z 350.10 |
| 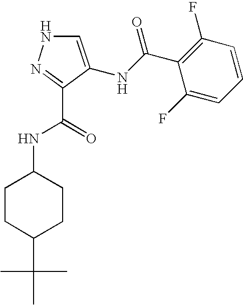 | Rₜ 5.05 min m/z 405.14 |
| 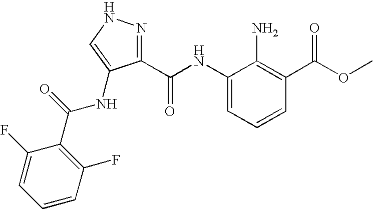 | Rₜ 2.87 min m/z 416.07 |
| 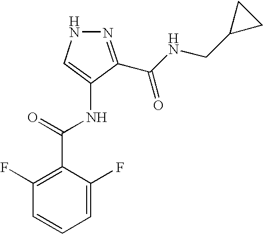 | Rₜ 3.41 min m/z 321.03 |
| 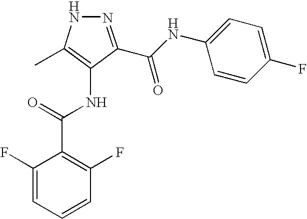 | Rₜ 3.42 min m/z 375.05 |
| 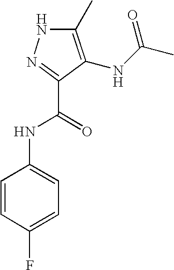 | Rₜ 2.37 min m/z 277.04 |
| 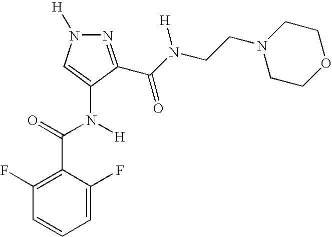 | [M + H]⁺ 380 Rₜ 1.42 |
| 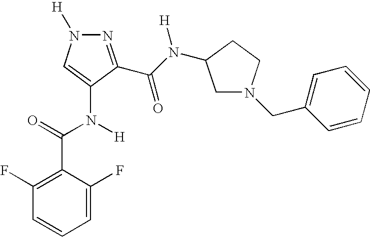 | [M + H]⁺ 426 Rₜ 1.93 |
| 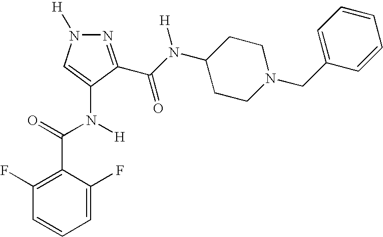 | [M + H]⁺ 440 Rₜ 1.87 |
| 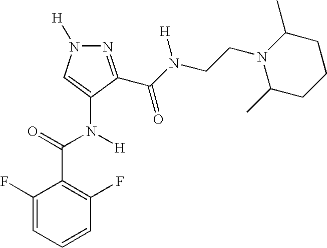 | [M + H]⁺ 406 Rₜ 2.78 |
| 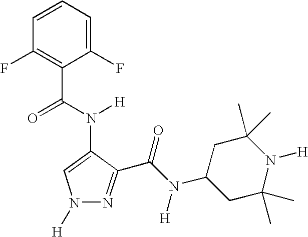 | [M + H]⁺ 406 Rₜ 2.55 |
| 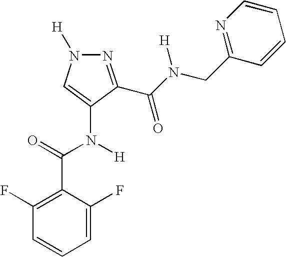 | [M + H]⁺ 358 Rₜ 1.98 |
| 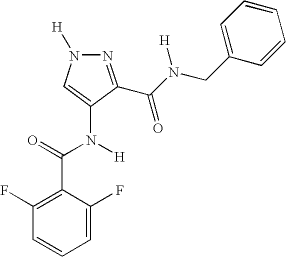 | [M + H]⁺ 357 Rₜ 3.37 |
| 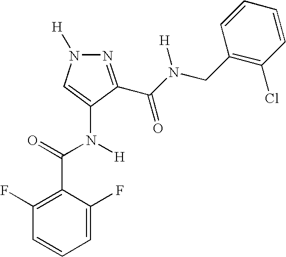 | [M + H]⁺ 391 Rₜ 3.16 |
| 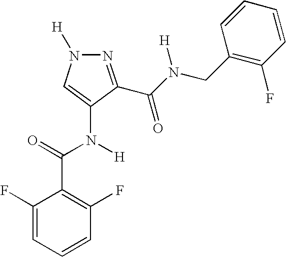 | [M + H]⁺ 375 Rₜ 3.02 |
| 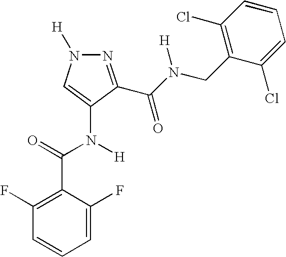 | [M + H]⁺ 425 Rₜ 3.27 |
| 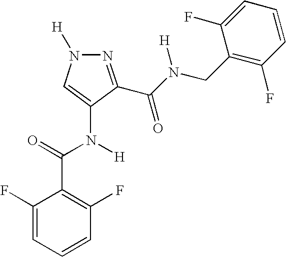 | [M + H]⁺ 393 Rₜ 3.01 |
| 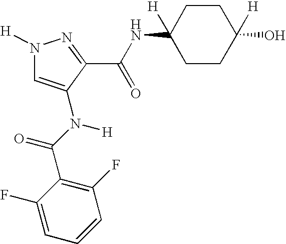 | [M + H]⁺ 365 Rₜ 2.22 |
| 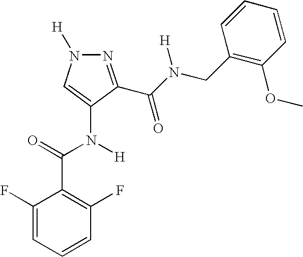 | [M + H]⁺ 387 Rₜ 3.05 |
| 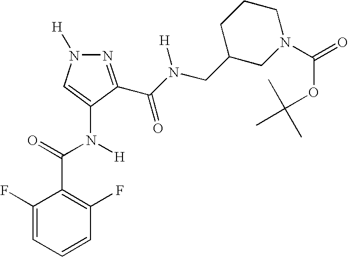 | [M + H]⁺ 464 Rₜ 3.17 |
| 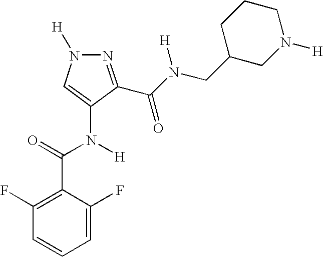 | [M + H]⁺ 364 Rₜ 1.76 |
| 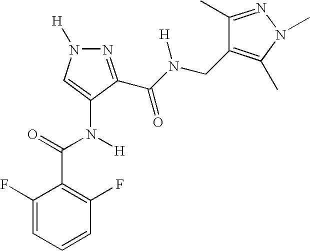 | [M + H]⁺ 389 Rₜ 2.36 |
| 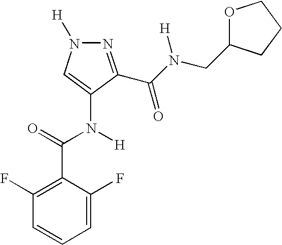 | [M + H]⁺ 351 Rₜ 2.55 |
| 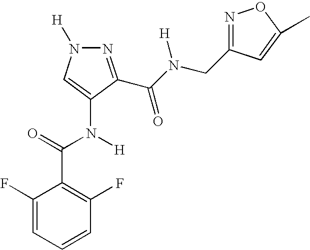 | [M + H]⁺ 362 Rₜ 2.63 |
| 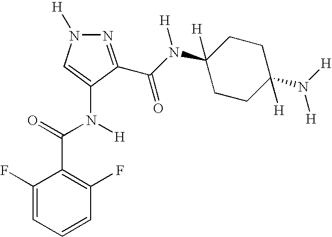 | [M + H]⁺ 364 Rₜ 1.75 |
| 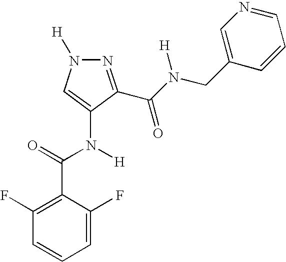 | [M + H]⁺ 358 Rₜ 3.2 |
| 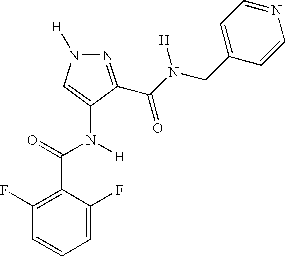 | [M + H]⁺ 358 Rₜ 1.77 |
| 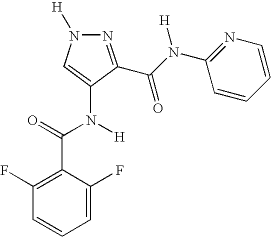 | [M + H]⁺ 344 Rₜ 2.71 |
| 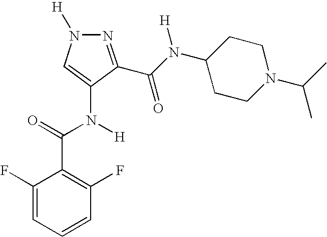 | [M + H]⁺ 392 Rₜ 2.57 |
| 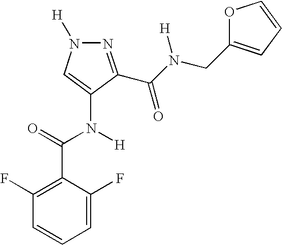 | [M + H]⁺ 347 Rₜ 2.8 |
| 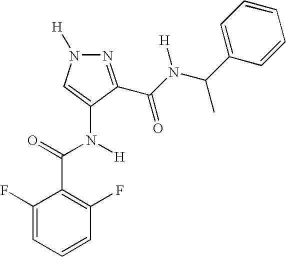 | [M + H]⁺ 371 Rₜ 3.1 |
| 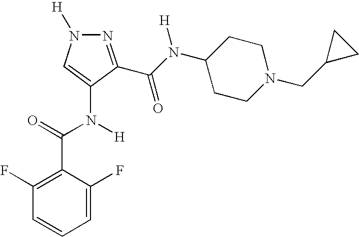 | [M + H]⁺ 404 Rₜ 2.7 |
| 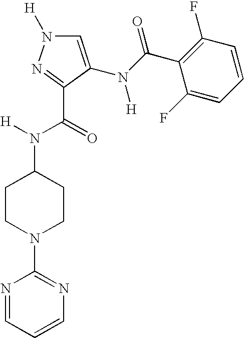 | [M + H]⁺ 428 Rₜ 2.63 |
| 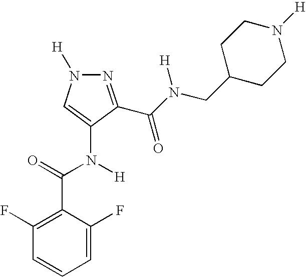 | [M + H]⁺ 364 Rₜ 1.75 |
| 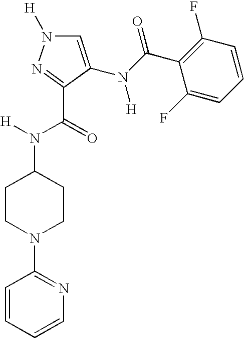 | [M + H]⁺ 427 Rₜ 2.71 |
| 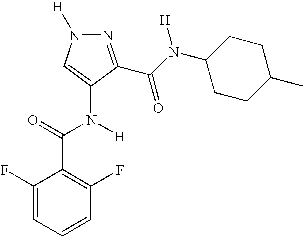 | [M + H]⁺ 363 Rₜ 3.34 |
| 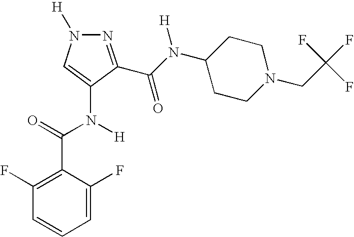 | [M + H]⁺ 432 Rₜ 2.63 |
| 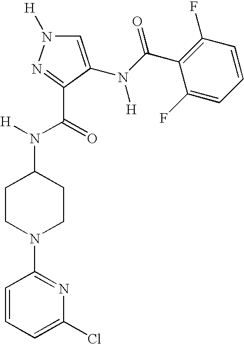 | [M + H]⁺ 461 Rₜ 3.3 |
| 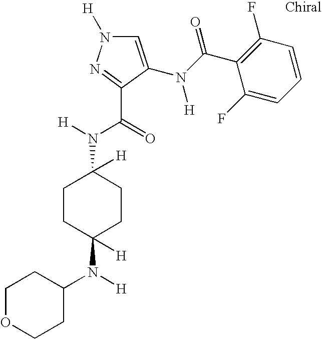 | [M + H]⁺ 448 Rₜ 1.87 |
| 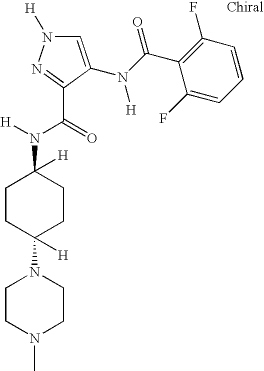 | [M + H]⁺ 447 Rₜ 1.65 |
| 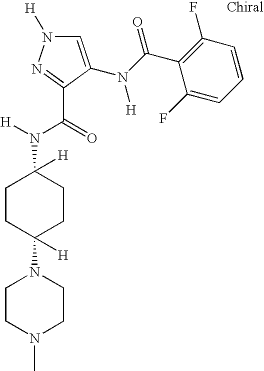 | [M + H]⁺ 447 Rₜ 1.72 |
| 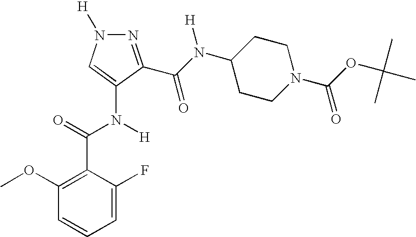 | [M + H]⁺ 462 Rₜ 2.97 |
| 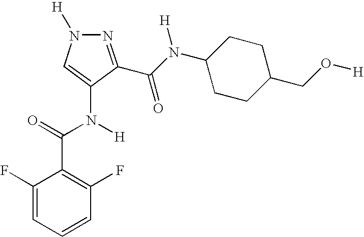 | [M + H]⁺ 379 Rₜ 2.45 |
| 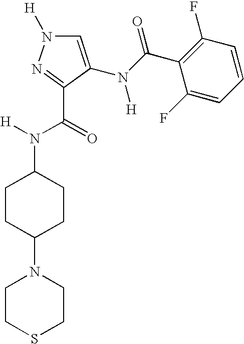 | [M + H]⁺ 450 Rₜ 1.97 |
| 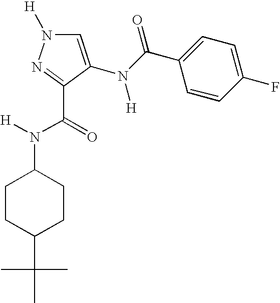 | [M + H]⁺ 387 Rₜ 3.83 |
| 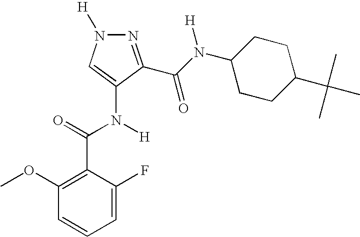 | [M + H]⁺ 417 Rₜ 3.65 |
| 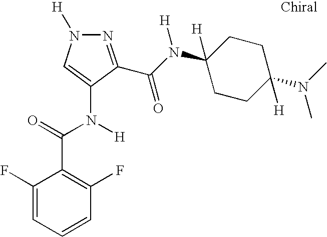 | [M + H]⁺ 392 Rₜ 1.85 |
| 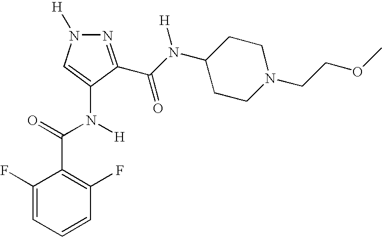 | [M + H]⁺ 408 Rₜ 1.82 |
| 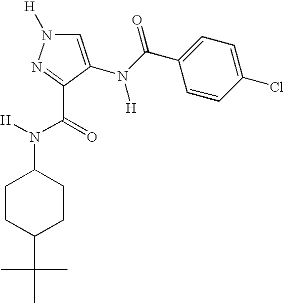 | [M + H]⁺ 403 Rₜ 4.02 |
| 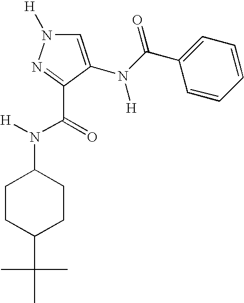 | [M + H]⁺ 369 Rₜ 3.78 |
| 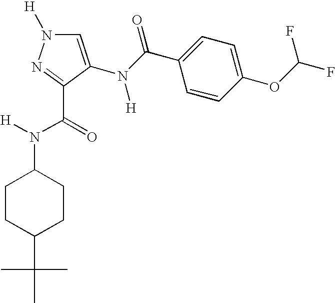 | [M + H]⁺ 435 Rₜ 3.83 |
| 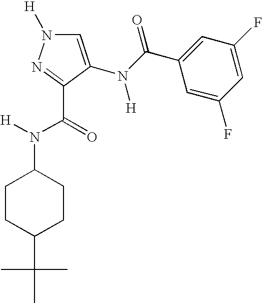 | [M + H]⁺ 405 Rₜ 3.96 |
| 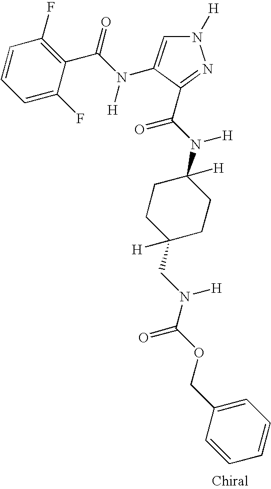 | [M + H]⁺ 512 Rₜ 3.1 |
| 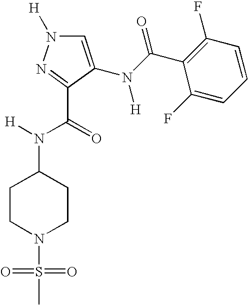 | [M + H]⁺ 428 Rₜ 2.45 |
| 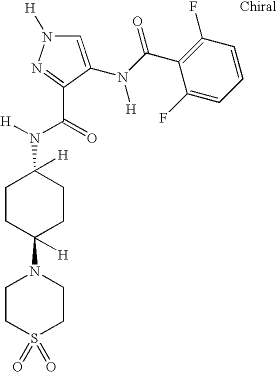 | [M + H]⁺ 482 Rₜ 1.96 |
| 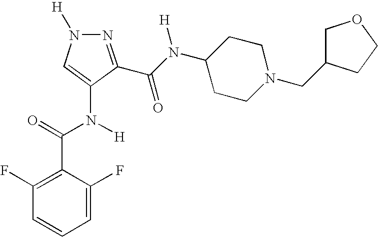 | [M + H]⁺ 434 Rₜ 2.3 |
| 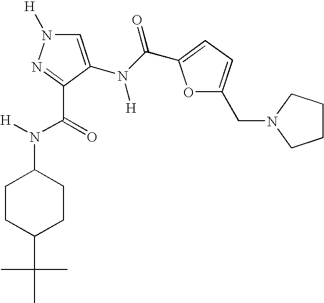 | [M + H]⁺ 442 Rₜ 2.39 |
| 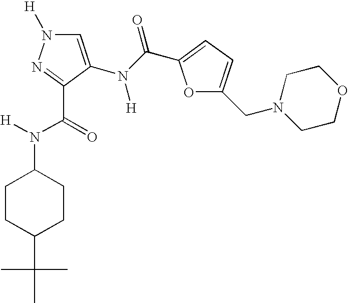 | [M + H]⁺ 458 Rₜ 2.26 |
| 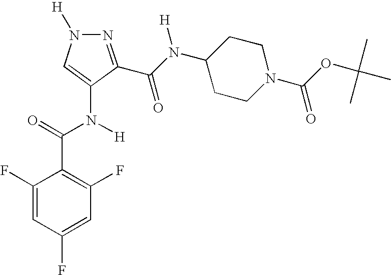 | [M + H]⁺ 468 Rₜ 3.07 |
| 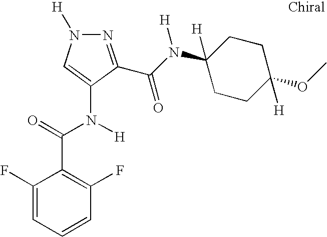 | [M + H]⁺ 379 Rₜ 2.6 |
| 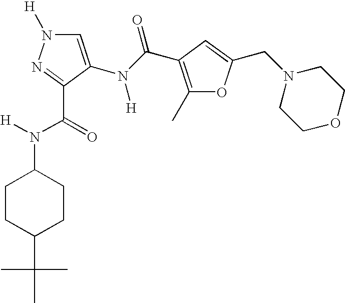 | [M + H]⁺ 472 Rₜ 2.40 |
| 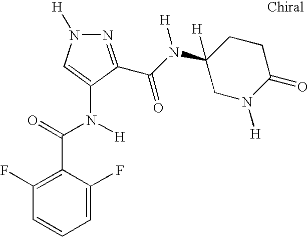 | [M + H]⁺ 364 Rₜ 2.1 |
| 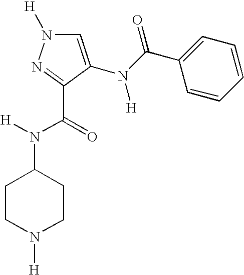 | [M + H]⁺ 314 Rₜ 1.78 |
| 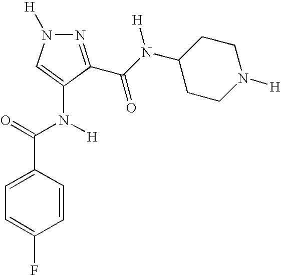 | [M + H]⁺ 332 Rₜ 1.89 |
| 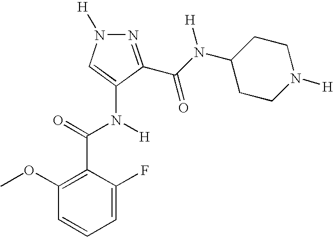 | [M + H]⁺ 362 Rₜ 1.78 |
| 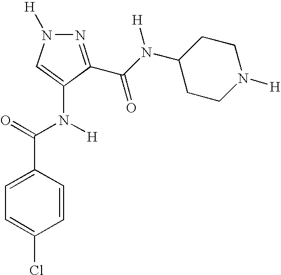 | [M + H]⁺ 348 Rₜ 2.01 |
| 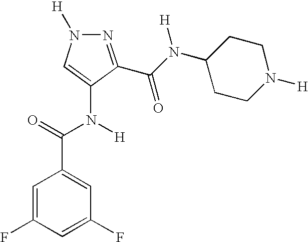 | [M + H]⁺ 350 Rₜ 1.97 |
| 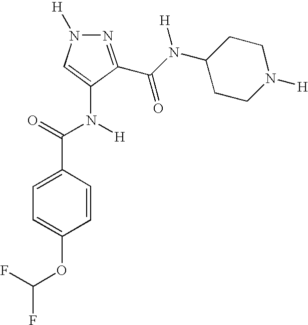 | [M + H]⁺ 380 Rₜ 2.01 |
| 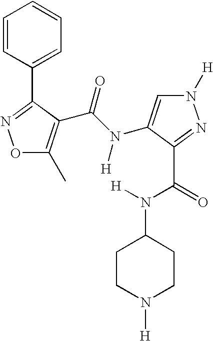 | [M + H]⁺ 395 Rₜ 1.94 |
| 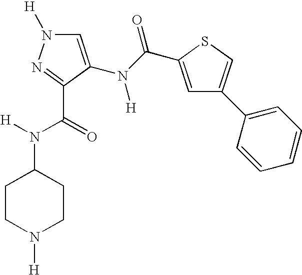 | [M + H]⁺ 396 Rₜ 2.11 |
| 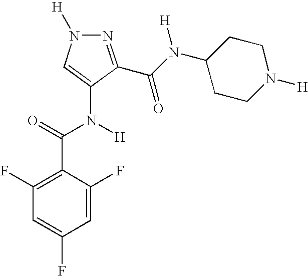 | [M + H]⁺ 368 Rₜ 1.76 |
| 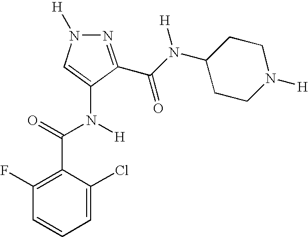 | [M + H]⁺ 366 Rₜ 1.78 |
| 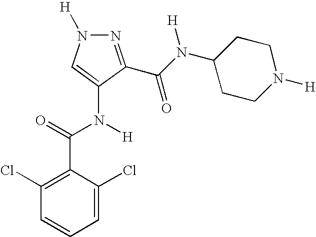 | [M + H]⁺ 383 Rₜ 1.87 |
| 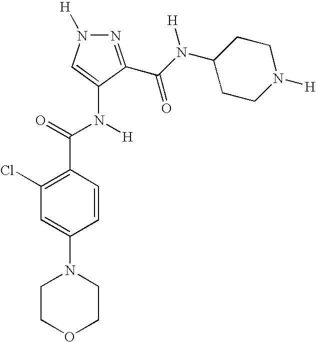 | [M + H]⁺ 433 Rₜ 1.89 |
| 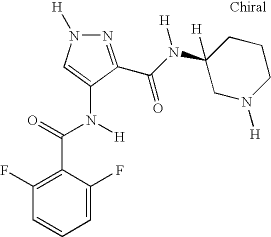 | [M + H]⁺ 350 Rₜ 1.76 |
| 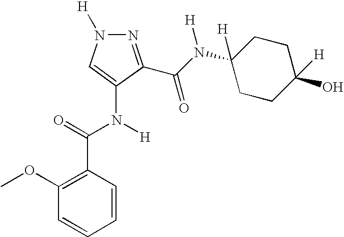 | [M + H]⁺ 359 Rₜ 2.29 |
| 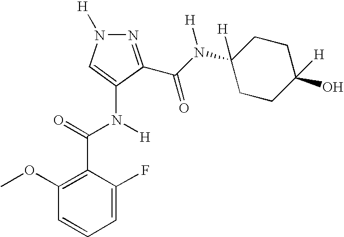 | [M + H]⁺ 377 Rₜ 2.22 |
| 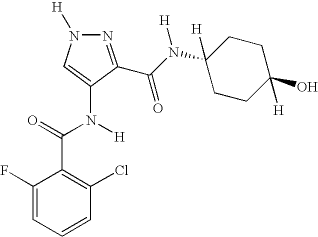 | [M + H]⁺ 381 Rₜ 2.34 |
| 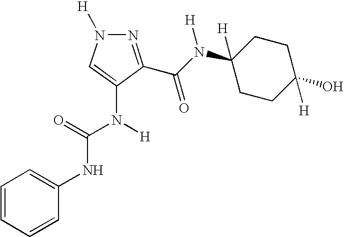 | [M + H]⁺ 344 Rₜ 2.28 |
| 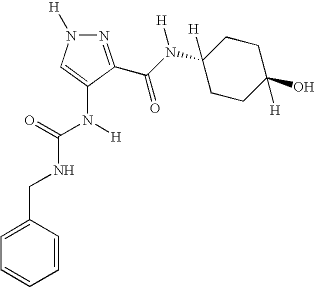 | [M + H]⁺ 358 Rₜ 2.22 |
| 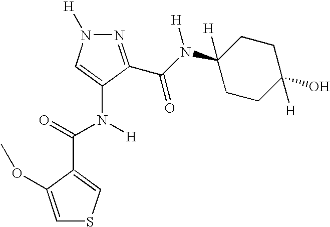 | [M + H]⁺ 365 Rₜ 2.21 |
| 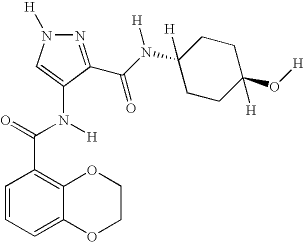 | [M + H]⁺ 387 Rₜ 2.29 |
| 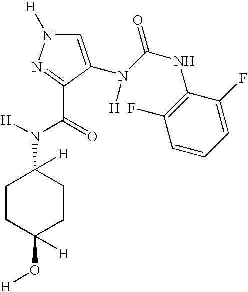 | [M + H]⁺ 380 Rₜ 2.17 |
| 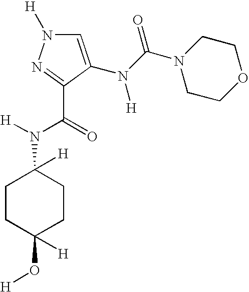 | [M + H]⁺ 338 Rₜ 1.68 |
| 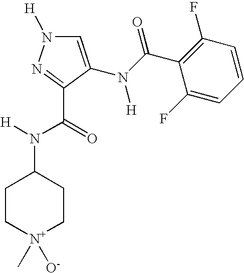 | [M + H]⁺ 380 Rₜ 1.83 |
| 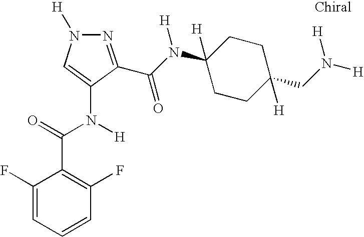 | [M + H]⁺ 378 Rₜ 1.78 |
| 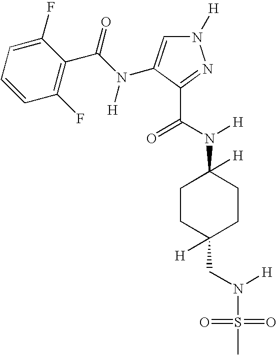 | [M + H]⁺ 456 Rₜ 2.54 |
| 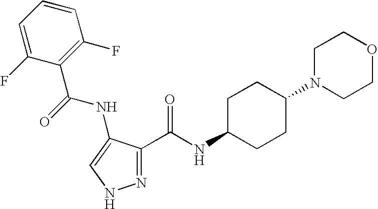 | [M + H]⁺ 434 Rₜ 1.97 |
| 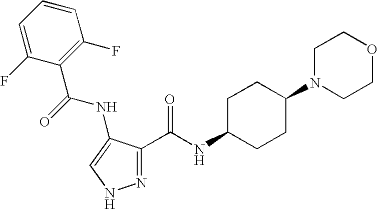 | [M + H]⁺ 434 Rₜ 2.03 |
| 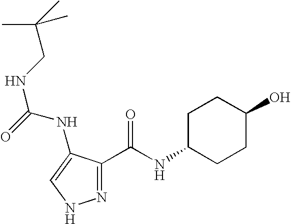 | [M + H]⁺ 338 Rₜ 2.28 |
| 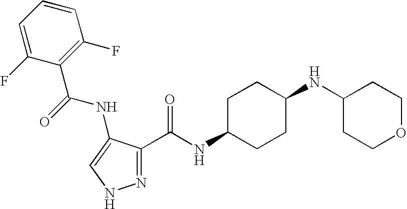 | [M + H]⁺ 448 Rₜ 1.97 |
| 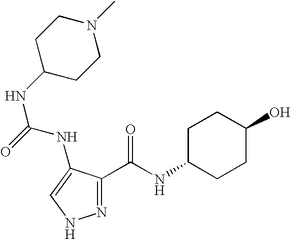 | [M + H]⁺ 365 Rₜ 0.34 |
| 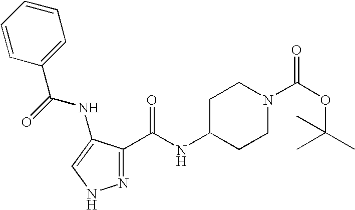 | [M + H]⁺ 414.13 Rₜ 3.05 |
| 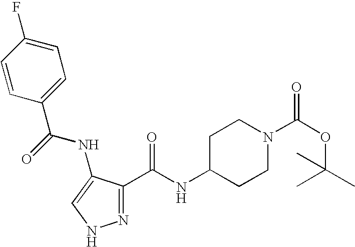 | [M + H]⁺ 432.12 Rₜ 3.12 |
| 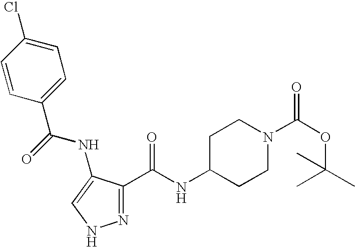 | [M + H]⁺ 448.06 Rₜ 3.33 |
| 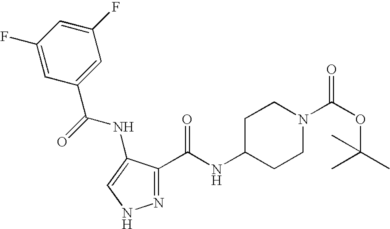 | [M + H]⁺ 450.08 Rₜ 3.29 |
| 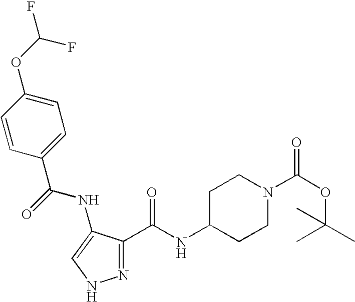 | [M + H]⁺ 480.05 Rₜ 3.18 |
| 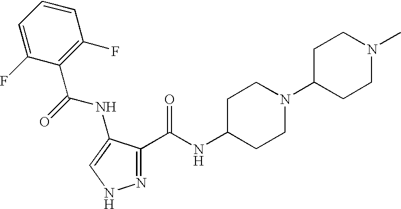 | [M + H]⁺ 447 Rₜ 2.01 |
| 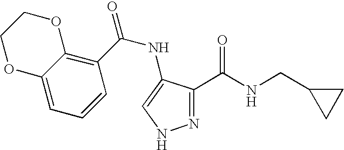 | [M + H]⁺ 343.05 Rₜ 3.38 (polar method) |
| 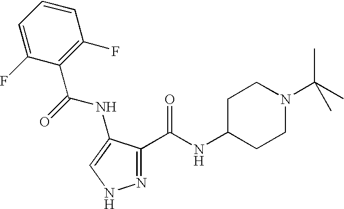 | [M + H]⁺ 406 Rₜ 1.85 |
| 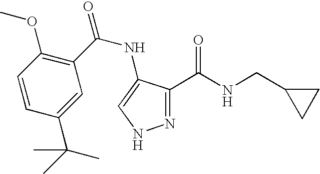 | [M + H]⁺ 371.09 Rₜ 3.27 (polar method) |
| 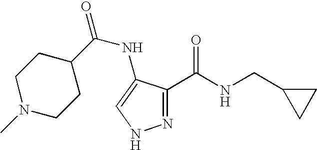 | [M + H]⁺ 306.06 Rₜ 1.53 |
| 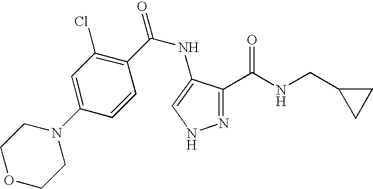 | [M + H]⁺ 403.98 Rₜ 2.78 |
| 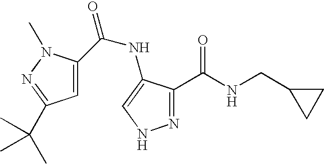 | [M + H]⁺ 345.05 Rₜ 3.03 |
| 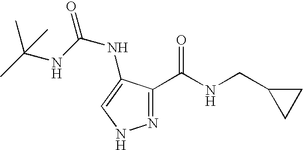 | [M + H]⁺ 280.05 Rₜ 3.75 (basic method) |
| 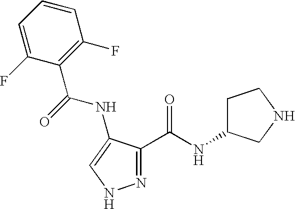 | [M + H]⁺ 336 Rₜ 1.67 |
| 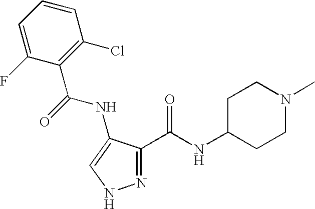 | [M + H]⁺ 380.05 Rₜ 1.78 |
| 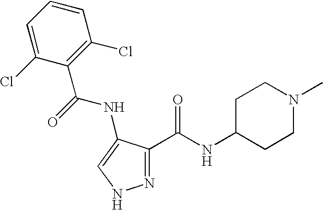 | [M + H]⁺ 396.02 Rₜ 1.86 |
| 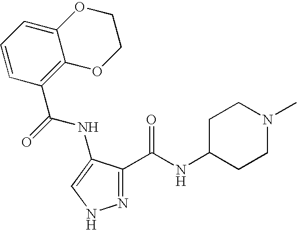 | [M + H]⁺ 386.10 Rₜ 1.88 |
| 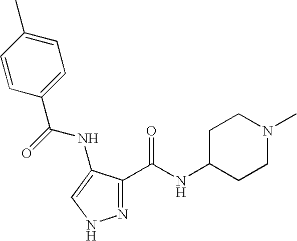 | [M + H]⁺ 342.10 Rₜ 1.95 |
| 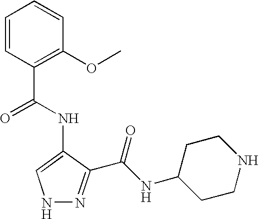 | [M + H]⁺ = 344 Rₜ = 1.87 |
| 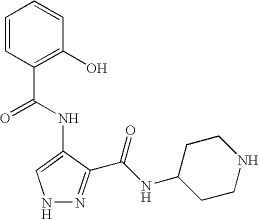 | [M + H]⁺ = 330 Rₜ = 1.80 |
| 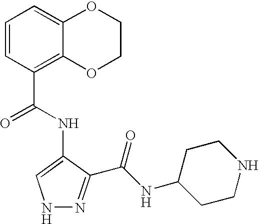 | [M + H]⁺ = 372 Rₜ = 1.87 |
| 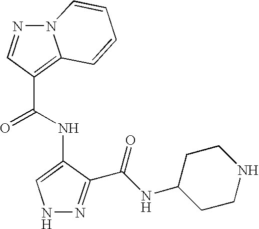 | [M + H]⁺ = 354 Rₜ = 1.77 |
| 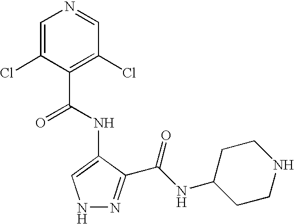 | [M + H]⁺ = 383/385 Rₜ = 1.72 |
| 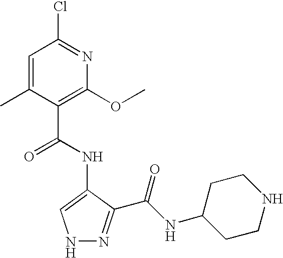 | [M + H]⁺ = 393/395 Rₜ = 1.86 |
| 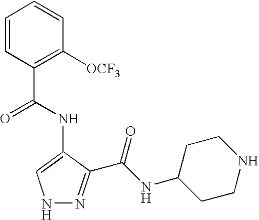 | [M + H]⁺ = 398 Rₜ = 1.94 |
| 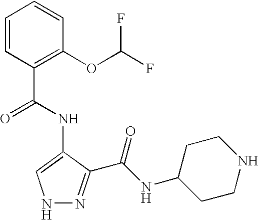 | [M + H]⁺ = 330 Rₜ = 1.80 |
| 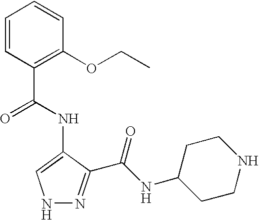 | [M + H]⁺ = 358 Rₜ = 1.89 |
| 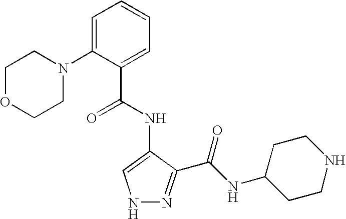 | [M + H]⁺ = 399 Rₜ = 1.88 |
| 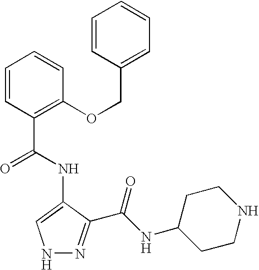 | [M + H]⁺ = 420 Rₜ = 2.13 |
| 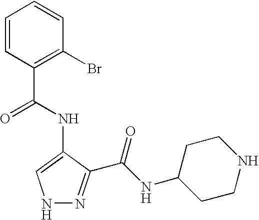 | [M + H]⁺ = 392/394 Rₜ = 1.84 |
| 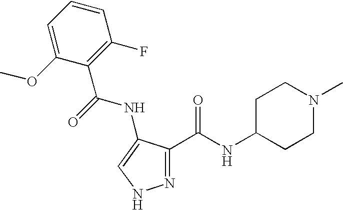 | [M + H]⁺ 376.14 Rₜ 1.78 |
| 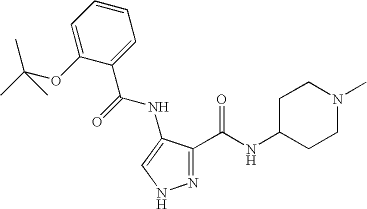 | [M + H]⁺ 400.17 Rₜ 2.08 |
| 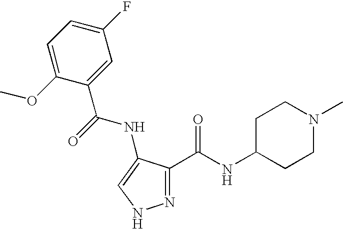 | [M + H]⁺ 376.15 Rₜ 1.92 |
| 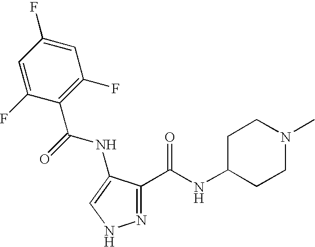 | [M + H]⁺ 382.12 Rₜ 1.77 |
| 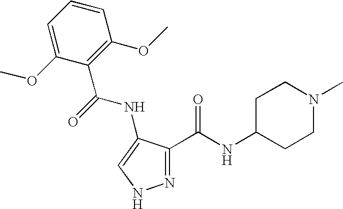 | [M + H]⁺ 388.18 Rₜ 1.73 |
| 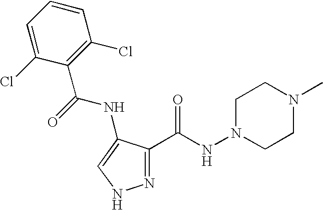 | [M + H]⁺ = 397/399 Rₜ = 1.83 |
| 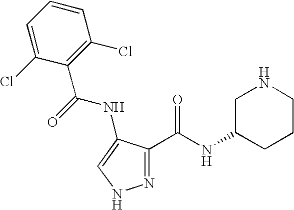 | [M + H]⁺ 382.02 Rₜ 1.82 |
| 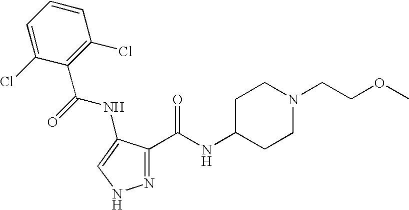 | [M + H]⁺ 440.22 Rₜ 1.92 |
| 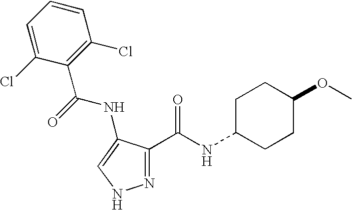 | [M + H]⁺ 411.20 Rₜ 2.97 |
| 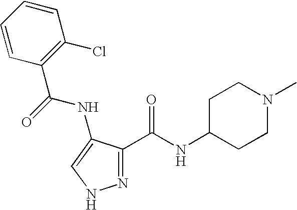 | [M + H]⁺ 362.11 Rₜ 1.91 |
| 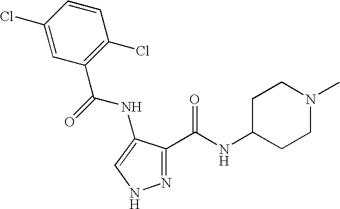 | [M + H]⁺ 396.08 Rₜ 2.06 |
| 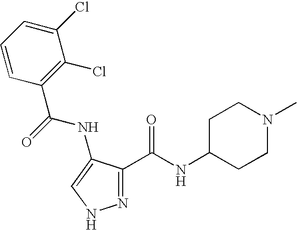 | [M + H]⁺ 396.06 Rₜ 2.04 |
| 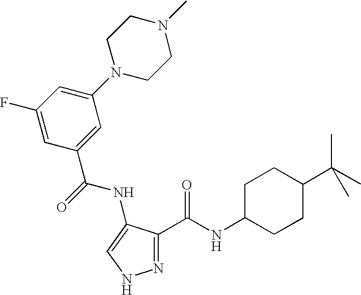 | [M + H]⁺ 485 Rₜ 2.59 |
| 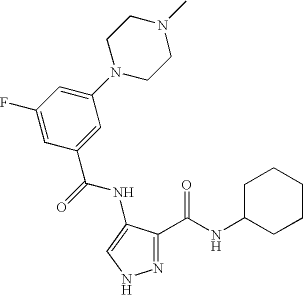 | [M + H]⁺ 429 Rₜ 2.25 |
| 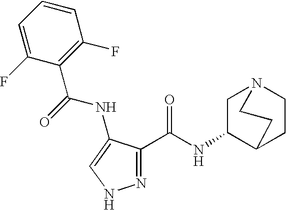 | [M + H]⁺ = 376 Rₜ = 1.85 |
| 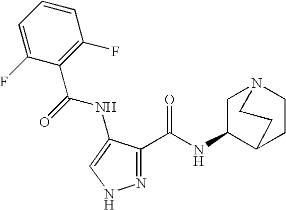 | [M + H]⁺ = 376 Rₜ = 1.87 |
| 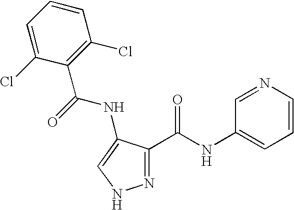 | [M + H]⁺ = 376/378 Rₜ = 2.23 |
| 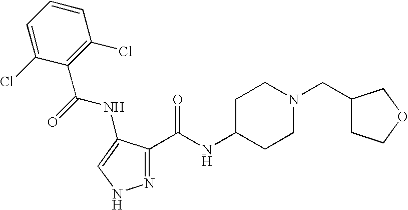 | [M + H]⁺ = 466/468 Rₜ = 1.98 |
| 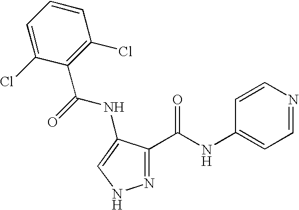 | [M + H]⁺ = 376/378 Rₜ = 2.09 |
| 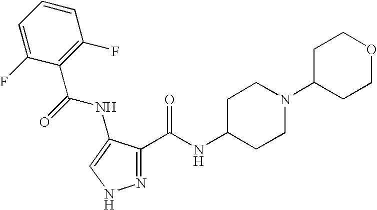 | [M + H]⁺ = 434 Rₜ = 1.82 |
| 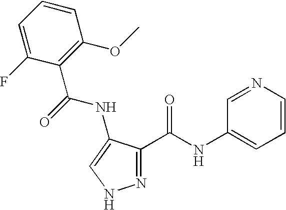 | [M + H]⁺ = 356 Rₜ = 2.11 |
| 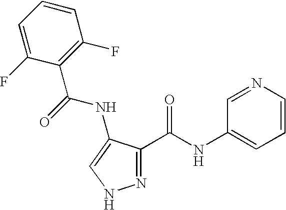 | [M + H]⁺ = 344 Rₜ = 2.09 |

### EQUIVALENTS

The foregoing examples are presented for the purpose of illustrating the invention and should not be construed as imposing any limitation on the scope of the invention. It will readily be apparent that numerous modifications and alterations may be made to the specific embodiments of the invention described above and illustrated in the examples without departing from the principles underlying the invention. All such modifications and alterations are intended to be embraced by this application.

1-87. (canceled)

88. A method of treating pain in a patient, which method comprises administering to the patient a therapeutically effective amount of a compound of formula (Ib): 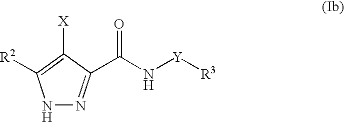
or salts or tautomers or N-oxides thereof;
wherein
X is a group R¹-A-NR⁴—;
A is a bond, C═O, NR^{g}(C═O) or O(C═O) wherein R^{g} is hydrogen or C₁₋₄ hydrocarbyl optionally substituted by hydroxy or C₁₋₄ alkoxy;
Y is a bond or an alkylene chain of 1, 2 or 3 carbon atoms in length;
R¹ is a carbocyclic or heterocyclic group having from 3 to 12 ring members; or a C₁₋₈ hydrocarbyl group optionally substituted by one or more substituents selected from fluorine, hydroxy, C₁₋₄ hydrocarbyloxy, amino, mono- or di-C₁₋₄ hydrocarbylamino, and carbocyclic or heterocyclic groups having from 3 to 12 ring members, and wherein 1 or 2 of the carbon atoms of the hydrocarbyl group may optionally be replaced by an atom or group selected from O, S, NH, SO, SO₂;
R² is hydrogen; halogen; C₁₋₄ alkoxy; or a C₁₋₄ hydrocarbyl group optionally substituted by halogen, hydroxyl or C₁₋₄ alkoxy;
R³ is selected from carbocyclic and heterocyclic groups having from 3 to 12 ring members; and
R⁴ is hydrogen or a C₁₋₄ hydrocarbyl group optionally substituted by halogen, hydroxyl or C₁₋₄ alkoxy.

89. A method according to claim 88 wherein R¹ is a carbocyclic or heterocyclic group having from 3 to 12 ring members wherein the carbocyclic and heterocyclic groups are optionally substituted by one or more substituent groups R¹⁰ selected from halogen, hydroxy, trifluoromethyl, cyano, nitro, carboxy, amino, mono- or di-C₁₋₄ hydrocarbylamino, carbocyclic and heterocyclic groups having from 3 to 12 ring members; a group R^{a}—R^{b} wherein R^{a} is a bond, O, CO, X¹C(X²), C(X²)X¹, X¹C(X²)X¹, S, SO, SO₂, NR^{c}, SO₂NR^{c} or NR^{c}SO₂; and R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl; and
X¹ is O, S or NR^{c} and X² is ═O, ═S or ═NR^{c}.

90. A method according to claim 88 wherein the compound has formula (II′): 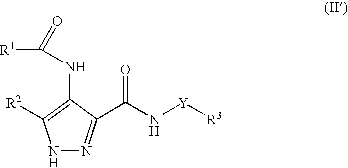
or salts or tautomers or N-oxides thereof.

91. A method according to claim 88 wherein the compound has formula (IV): 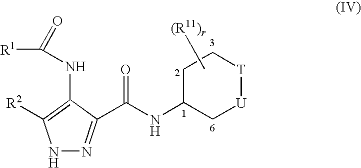
or salts or tautomers or N-oxides thereof;
an optional second bond may be present between carbon atoms numbered 1 and 2;
one of U and T is selected from CH₂, CHR¹³, CR¹¹R¹³, NR¹⁴, N(O)R¹⁵, O and S(O)ₜ; and the other of U and T is selected from, NR¹⁴, O, CH₂, CHR¹¹, C(R¹¹)₂, and C═O; r is 0, 1, 2, 3 or 4; t is 0, 1 or 2;
R¹¹ is selected from hydrogen, halogen, C₁₋₃ alkyl and C₁₋₃ alkoxy;
R¹³ is selected from hydrogen, NHR¹⁴, NOH, NOR¹⁴ and R^{a}—R^{b};
R¹⁴ is selected from hydrogen and R^{d}—R^{b};
R^{a} is a bond, O, CO, X¹C(X²), C(X²)X¹, X¹C(X²)X¹, S, SO, SO₂, NR^{c}, SO₂NR^{c} or NR^{c}SO₂;
R^{b} is selected from hydrogen, carbocyclic and heterocyclic groups having from 3 to 12 ring members, and a C₁₋₈ hydrocarbyl group optionally substituted by one or more substituents selected from hydroxy, oxo, halogen, cyano, nitro, carboxy, amino, mono- or di-C₁₋₄ hydrocarbylamino, carbocyclic and heterocyclic groups having from 3 to 12 ring members and wherein one or more carbon atoms of the C₁₋₈ hydrocarbyl group may optionally be replaced by O, S, SO, SO₂, NR^{c}, X¹C(X²), C(X²)X¹ or X¹C(X²)X¹;
R^{c} is selected from hydrogen and C₁₋₄ hydrocarbyl;
R^{d} is selected from a bond, CO, C(X²)X¹, SO₂ and SO₂NR^{c};
X¹ is O, S or NR^{c} and X² is ═O, ═S or ═NR^{c}; and
R¹⁵ is selected from C₁₋₄ saturated hydrocarbyl optionally substituted by hydroxy, C₁₋₂ alkoxy, halogen or a monocyclic 5- or 6-membered carbocyclic or heterocyclic group, provided that U and T cannot be 0 simultaneously.

92. A method according to claim 91 wherein the compound has formula (IVa): 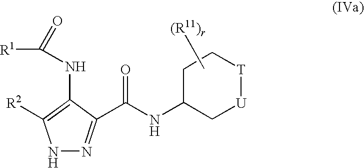
or salts or tautomers or N-oxides thereof;
wherein one of U and T is selected from CH₂, CHR¹³, CR¹¹R¹³, NR¹⁴, N(O)R¹⁵, O and S(O)ₜ; and
the other of U and T is selected from CH₂, CHR¹¹, C(R¹¹)₂, and C═O; r is 0, 1 or 2; t is 0, 1 or 2;
R¹¹ is selected from hydrogen and C₁₋₃ alkyl;
R¹³ is selected from hydrogen and R^{a}—R^{b};
R¹⁴ is selected from hydrogen and R^{d}—R^{b};
R^{d} is selected from a bond, CO, C(X²)X¹, SO₂ and SO₂NR^{c}; and
R¹⁵ is selected from C₁₋₄ saturated hydrocarbyl optionally substituted by hydroxy, C₁₋₂ alkoxy, halogen or a monocyclic 5- or 6-membered carbocyclic or heterocyclic group.

93. A method according to claim 92 wherein the compound has formula (Va): 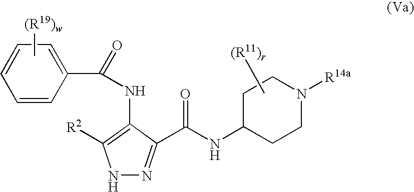
or salts or tautomers or N-oxides thereof;
wherein R^{14a} is selected from hydrogen, C₁₋₄ alkyl optionally substituted by fluoro, cyclopropylmethyl, phenyl-C₁₋₂ alkyl, C₁₋₄ alkoxycarbonyl, phenyl-C₁₋₂ alkoxycarbonyl, C₁₋₂-alkoxy-C₁₋₂ alkyl, and C₁₋₄ alkylsulphonyl, wherein the phenyl moieties when present are optionally substituted by one to three substituents selected from fluorine, chlorine, C₁₋₄ alkoxy optionally substituted by fluoro or C₁₋₂-alkoxy, and C₁₋₄ alkyl optionally substituted by fluoro or C₁₋₂-alkoxy;
w is 0, 1, 2 or 3;
R² is hydrogen or methyl;
R¹¹ and r are as defined in claim 92; and
R¹⁹ is selected from fluorine; chlorine; C₁₋₄ alkoxy optionally substituted by fluoro or C₁₋₂-alkoxy; and C₁₋₄ alkyl optionally substituted by fluoro or C₁₋₂-alkoxy.

94. A method according to claim 93 wherein the compound has formula (VIa): 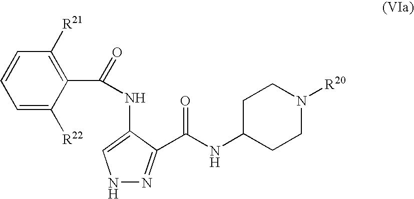
or salts or tautomers or N-oxides thereof;
wherein R²⁰ is selected from hydrogen and methyl;
R²¹ is selected from fluorine and chlorine; and
R²² is selected from fluorine, chlorine and methoxy; or
one of R²¹ and R²² is hydrogen and the other is selected from chlorine, methoxy, ethoxy, difluoromethoxy, trifluoromethoxy and benzyloxy.

95. A method according to claim 94 wherein the compound has formula (VIb): 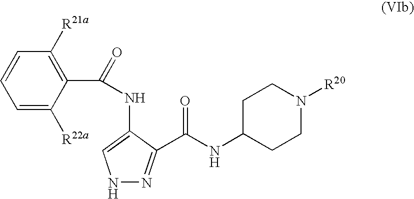
or salts or tautomers or N-oxides thereof;
wherein R²⁰ is selected from hydrogen and methyl;
R^{21a} is selected from fluorine and chlorine; and
R^{22a} is selected from fluorine, chlorine and methoxy.

96. A method according to claim 95 wherein the compound of the formula (VIb) is 4-(2,6-dichloro-benzoylamino)-1H-pyrazole-3-carboxylic acid piperidin-4-ylamide or a salt thereof.

97. A method according to claim 88 wherein the compound has formula (I′″): 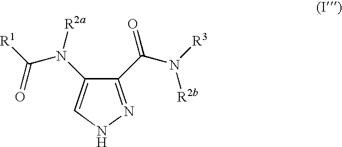
or salts, tautomers, and N-oxides thereof;
wherein:
R¹ is 2,6-dichlorophenyl;
R^{2a} and R^{2b} are both hydrogen;
and R³ is a group: 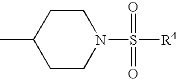
where R⁴ is C₁₋₄ alkyl.

98. A method according to claim 97 wherein R⁴ is methyl and the compound is 4-(2,6-dichloro-benzoylamino)-1H-pyrazole-3-carboxylic acid (1-methanesulphonyl-piperidin-4-yl)-amide.

99. A method for reducing or eliminating pain in a patient suffering from pain, which method comprises administering to the patient an effective pain-reducing or pain-eliminating amount of a compound as defined in claim 88 or a salt or tautomer or N-oxide thereof.

100. A method for treating of any one or more of nociception, somatic pain, visceral pain, acute pain, chronic pain, hyperalgesia, allodynia, post operative pain, pain due to hypersensivity, headache, inflammatory pain (rheumatic, dental, dysmenorrhoea or infection), neurological pain, musculoskeletal pain, cancer related pain or vascular pain, which method comprises administering to the patient a therapeutically effective amount of a compound as defined in claim 88 or a salt or tautomer or N-oxide thereof.

101. A method for prophylaxis or treatment of stroke in a patient, which method comprises administering to the patient a therapeutically effective amount of a compound as defined in claim 88 or a salt or tautomer or N-oxide thereof.

102. A method for prophylaxis or treatment of stroke in a patient, which method comprises administering to the patient a therapeutically effective amount of a compound as defined in claim 97 or a salt or tautomer or N-oxide thereof.

103. A method of preventing or reducing neuronal damage in a patient suffering from stroke, which method comprises administering to the patient an effective neuroprotective amount of a compound as defined in claim 88 or a salt or tautomer or N-oxide thereof.

104. A method for preventing or reducing the risk of stroke in patients at risk for stroke, which method comprises administering to the patient an effective therapeutic amount of compound as defined in claim 88 or a salt or tautomer or N-oxide thereof.

105. A method for prophylaxis or treatment of polycystic kidney disease in a patient, which method comprises administering to the patient a therapeutically effective amount of a compound as defined in claim 88 or a salt or tautomer or N-oxide thereof.

106. A method for prophylaxis or treatment of polycystic kidney disease in a patient, which method comprises administering to the patient a therapeutically effective amount of a compound as defined in claim 97 or a salt or tautomer or N-oxide thereof.

107. A method for preventing or slowing down the progression of polycystic kidney disease in a patient, which method comprises administering to the patient a therapeutically effective amount of a compound as defined in claim 88 or a salt or tautomer or N-oxide thereof.

108. A method for the treatment of progressive renal insufficiency associated with the progression of cystic kidney disease in a patient, which method comprises administering to the patient a therapeutically effective amount of a compound as defined in claim 88 or a salt or tautomer or N-oxide thereof.

109. A method for prophylaxis or treatment (including alleviating or reducing) the incidence of a disease state or condition mediated by a cyclin dependent kinase 5, which method comprises administering to a subject in need thereof a compound as defined in claim 88 or a salt or tautomer or N-oxide thereof.

110. A pharmaceutical composition for the treatment of a disease selected from stroke, pain and polycystic kidney disease, comprising an effective amount of a compound as defined in claim 88 or a salt or tautomer or N-oxide thereof in admixture with a pharmaceutically acceptable carrier.
